SEQUENCE LISTING (78 sequences)

SEQ ID NO: 1 - DNA, 1629 bp, Arabidopsis thaliana
cctgacgtagcacgtgtttgtgtcttgactgattcttctctcaagcttttttaatctctctctcttttcccacgtaattcccccaaatccattctttctagggttcgatctccctctctcaatcatgaaccttcttctcttctagaccccacaaagtttcccccttttatttgatcggcgacggagaagcctaagtctgatcccggaatgtctgtctccgagctcaaagaacgccacgccgtcgctacggagaccgttaataacctccgtgaccagcttagacagagacgcctccagctcctcgataccgatgtggcgaggtattcagcggcgcaaggacgtactcgggtgagcttcggagcaacggatctggtttgttgtcgtactcttcagggacacaccggaaaggtttattcattagattggacaccggagaggaaccggattgtcagtgcatctcaagatgggagattaatcgtgtggaatgctctaacgagtcagaaaactcatgctattaaactcccttgtgcatgggttatgacatgtgctttctctccaaatggtcagtcggttgcgtgtggtggattagacagtgtatgttctatctttagccttagctcaacggcggacaaggatggaactgtaccggtttcaagaatgctcactggtcacaggggatatgtttcgtgctgtcagtatgtcccaaatgaggatgcccaccttatcaccagttcaggtgatcaaacttgtatcttatgggatgtaactactggtctcaaaacttctgtttttggcggtgaatttcagtctggacatactgctgatgtactaagcgtctcaatcagtggatcaaacccaaactggtttatatctggttcatgcgattccacagcacggttgtgggacactcgtgctgcaagccgagcagtgcgtacctttcatggtcacgagggagatgttaatacggtcaagttctttccggatgggtatagatttgggactggatcagacgatggaacatgcaggctgtatgacataaggactggtcaccaactccaggtctatcagccacatggtgatggtgagaacggacctgtcacctccattgcattctctgtgtcagggagacttcttttcgctggctatgcgagcaacaacacttgctacgtttgggataccctcttgggagaggttgtattggatttgggattacagcaggattcacacaggaatagaataagctgtttggggttgtcagcagatggaagtgcattgtgtacaggaagttgggattcaaatctaaagatatgggcgtttggaggacacaggagagtgatttgaagaagatttaacgaaaagtaggagtcacgtctccagttgttggttaatatattctgtagtcgggaagtaaggttcggtttgtggaaggtgtttggtttgaaatagtggagtggttagaagaattaaacttccctttttgtagtgtgctttgatttatttatttcttcattgggaactaaactccttcaacacgctactcaatgtgaattctgtaatcaattgtgtacccaccagtctttactttactatcatctcttcatattgaacgcagaagataaaacgctacta

SEQ ID NO: 2 - protein, 377 aa, Arabidopsis thaliana
MSVSELKERHAVATETVNNLRDQLRQRRLQLLDTDVARYSAAQGRTRVSFGATDLVCCRTLQGHTGKVYSLDWTPERNRIVSASQDGRLIVWNALTSQKTHAIKLPCAWVMTCAFSPNGQSVACGGLDSVCSIFSLSSTADKDGTVPVSRMLTGHRGYVSCCQYVPNEDAHLITSSGDQTCILWDVTTGLKTSVFGGEFQSGHTADVLSVSISGSNPNWFISGSCDSTARLWDTRAASRAVRTFHGHEGDVNTVKFFPDGYRFGTGSDDGTCRLYDIRTGHQLQVYQPHGDGENGPVTSIAFSVSGRLLFAGYASNNTCYVWDTLLGEVVLDLGLQQDSHRNRISCLGLSADGSALCTGSWDSNLKIWAFGGHRRVI

SEQ ID NO: 3 - DNA, 1152 bp, Arabidopsis thaliana
atgggcttactctgcagtagaagtcgacatcatactgaagatactgatgagaatacacaggctgctgaaatcgaaagacggatagagcaagaagcaaaggctgaaaagcatattcggaagcttttgctacttggtgctggggaatctggaaaatctacaatttttaagcagataaaacttctattccaaacgggatttgatgaaggagaactaaagagctatgttccagtcattcatgccaatgtctatcagactataaaattattgcatgatggaacaaaggagtttgctcaaaatgaaacagattctgctaaatatatgttatcttctgaaagtattgcaattggggagaaactatctgagattggtggtaggttagactatccacgtcttaccaaggacatcgctgagggaatagaaacactatggaaggatcctgcaattcaggaaacttgtgctcgtggtaatgagcttcaggttcctgattgtacgaaatatctgatggagaacttgaagagactatcagatataaattatattccaactaaggaggatgtactttatgcaagagttcgcacaactggtgtcgtggaaatacagttcagccctgtgggagagaataaaaaaagtggtgaagtgtaccgattgtttgacgtgggtggacagagaaatgagaggaggaaatggattcatctgtttgaaggtgtaacagctgtgatattttgtgctgccatcagcgagtacgaccaaacgctctttgaggacgagcagaaaaacaggatgatggagaccaaggaattattcgactgggtcctgaaacaaccctgttttgagaaaacatccttcatgctgttcttgaacaagttcgacatatttgagaagaaagttcttgacgttccgttgaacgtttgcgagtggttcagagattaccaaccagtttcaagtgggaaacaagagattgagcatgcatacgagtttgtgaagaagaagtttgaggagttatattaccagaacacggcgccggatagagtggacagggtattcaaaatctacaggacgacggctttggaccagaagcttgtaaagaaaacgttcaagctcgtagatgagacactaagaaggagaaatttactggaggctggccttttatga

SEQ ID NO: 4 - protein, 383 aa, Arabidopsis thaliana
MGLLCSRSRHHTEDTDENTQAAEIERRIEQEAKAEKHIRKLLLLGAGESGKSTIFKQIKLLFQTGFDEGELKSYVPVIHANVYQTIKLLHDGTKEFAQNETDSAKYMLSSESIAIGEKLSEIGGRLDYPRLTKDIAEGIETLWKDPAIQETCARGNELQVPDCTKYLMENLKRLSDINYIPTKEDVLYARVRTTGVVEIQFSPVGENKKSGEVYRLFDVGGQRNERRKWIHLFEGVTAVIFCAAISEYDQTLFEDEQKNRMMETKELFDWVLKQPCFEKTSFMLFLNKFDIFEKKVLDVPLNVCEWFRDYQPVSSGKQEIEHAYEFVKKKFEELYYQNTAPDRVDRVFKIYRTTALDQKLVKKTFKLVDETLRRRNLLEAGLL

SEQ ID NO: 5 - DNA, 1540 bp, Solanum tuberosum
ctttctctttttctttcttcttcagaaaccctaattcaaaagcgaaaaaaaatcttgcgattttgtgaaaatcatgtttcaatttccttaaagaaatgtcagttgcggagctgaaagagcggcacatggccgctacacagactgtaaatgatctccgtgaaaaacttaagcagaagcgtctccaattactcgacacagatgtttctgggtatgcaaagacgcaaggtaaaactccggtaacgttcggcccaacagatctagtttgttgtaggatcctgcaaggacacactggaaaggtctattcactggactggactcctgaaaaaaatcgtatagtcagtgcatcccaagatggtagattaatagtgtggaatgctctcacaagccagaaaacccatgcaattaagcttccatgtgcttgggttatgacctgtgccttctctcctagtggacagtctgttgcttgtggcggccttgacagtgcctgctctatcttcaacttaaattcaccaattgataaggatgggatccatccagtatcgagaatgcttagtgggcataaggggtatgtgtcttcgtgtcagtatgttccggatgaggatactcacctaataactagttctggtgatcaaacatgtgtactttgggatataactactggcctaagaacttctgtgtttggaggtgagtttcaatctgggcacactgcagatgtatcaagtgtctcaattagttcatctaaccccaaactatttgtgtctgggtcctgtgacacaactgctcgactgtgggacacccgagttgctagtcgagctcaacgaacatttcatggacacgagagtgatgttactactgtaaagttcttccctgacggtaatagatttggaactggttcagatgatggcagctgcagattatttgacattaggactggacaccagctgcaagtatacaaccaaccgcatggtgacggtgacatccctcatgtgacttccattgcattttctatctcaggccgtcttctctttgtcgggtactctaatggtgattgttacgtgtgggacaccctattagcaaaggtggtcctaaacttaggatcagttcaaaactctcatgaagggcgaataagttgtctgggactgtcagctgatggaagtgccttatgtacaggaagttgggatacaaacctgaagatttgggcttttggaggacacagaagtgtgatctgagttatgaaacacctcattctgttatttatctcaagtccctcttcattctcattttctttcatggccagcctgtgggttcgcgatttcttttggcatcttcataacctgtagatctctttaattctagttaatatttcagtcagataaaccaaattgtttccacatgaatctgacataaattactagaccagcaccagttgtaaagaataacctgtttgttgtcaaaattgtctgatggtttcagctgcttatgtaattaaaattctttttaaaaaaaaatcttgaagaatgaaaacaagctttacttttgc

SEQ ID NO: 6 - protein, 377 aa, Solanum tuberosum
MSVAELKERHMAATQTVNDLREKLKQKRLQLLDTDVSGYAKTQGKTPVTFGPTDLVCCRILQGHTGKVYSLDWTPEKNRIVSASQDGRLIVWNALTSQKTHAIKLPCAWVMTCAFSPSGQSVACGGLDSACSIFNLNSPIDKDGIHPVSRMLSGHKGYVSSCQYVPDEDTHLITSSGDQTCVLWDITTGLRTSVFGGEFQSGHTADVSSVSISSSNPKLFVSGSCDTTARLWDTRVASRAQRTFHGHESDVTTVKFFPDGNRFGTGSDDGSCRLFDIRTGHQLQVYNQPHGDGDIPHVTSIAFSISGRLLFVGYSNGDCYVWDTLLAKVVLNLGSVQNSHEGRISCLGLSADGSALCTGSWDTNLKIWAFGGHRSVI

SEQ ID NO: 7 - DNA, 1524 bp, Solanum tuberosum
aaaaatcttgcgattttgtgaaaatcatgtttcaatttccttaaagaaatgtcagttgcggagctgaaagagcggcacatggccgctacacagactgtaaatgatctccgtgaaaaacttaagcagaagcgtctccaattactcgacacagatgtttctgggtatgcaaagaggcaaggtaaaagtccggtaacgttcggcccaacagatctagtttgttgtaggatcctgcaaggacacactggaaaggtctattcactggactggactcctgaaaaaaatcgtatagtcagtgcatcccaagatggtagattaatagtgtggaatgctctcacaagccagaaaacccatgcaattaagcttccatgtgcttgggttatgacctgtgccttctctcctagtggacagtctgttgcttgtggcggccttgacagtgcctgctctatcttcaacttaaattcaccaatcgataaggatgggatccatccagtatcgagaatgcttagtgggcataaggggtatgtgtcttcgtgtcagtatgttccggatgaggatactcacctaataactagttctggtgatcaaacatgtgtactttgggatataactactggcctaagaacttctgtgtttggaggtgagtttcaatctgggcacactgcagatgtattaagtgtctcaattagttcatctaaccccaaactgtttgtgtctgggtcctgtgacacaactgctcgactgtgggacacccgagttgctagtcgagctcaacgaacatttcatggacacgagagtgatgttaatactgtaaagttcttccctgacggtaatagatttggaactggttcagatgatggaagctgcagattatttgacattaggactggacaccagctgcaagtatacaaccaaccgcatggtgacggtgacatccctcatgtgacttccatggcattttctatctcaggccgtcttctctttgtcgggtactctaatggtgattgttacgtgtgggacaccctattagcaaaggtggtcctaaacttaggatcagttcaaaactctcatgaagggcgaataagttgtctgggactgtcagctgacggaagtgccttatgtacaggaagttgggatacaaacctgaagatttgggcttttggaggacacagaagtgtggtctgagttatgaaacacctcattctgttatttatctcaagtccctcttcattctcattttctttcatggccggcctgtgggttcgcgatttcttttggcatcttcataacctgtagatctctttaattctagttaatatttcagtcagataaaccaaattgtttccacatgaatctgacatatattactagaccagcaccagttgtaaagaataacctgtttgttgtcaaaattgtctgatggtttcagctgcttatgtaattaaaattctttttaaaaaaaatcttgaagaatgaaaacaagctttacttttgccaccatcaaaaaaaaaaaaaaaaaaaaaaaaa

SEQ ID NO: 8 - protein, 377 aa, Solanum tuberosum
MSVAELKERHMAATQTVNDLREKLKQKRLQLLDTDVSGYAKRQGKSPVTFGPTDLVCCRILQGHTGKVYSLDWTPEKNRIVSASQDGRLIVWNALTSQKTHAIKLPCAWVMTCAFSPSGQSVACGGLDSACSIFNLNSPIDKDGIHPVSRMLSGHKGYVSSCQYVPDEDTHLITSSGDQTCVLWDITTGLRTSVFGGEFQSGHTADVLSVSISSSNPKLFVSGSCDTTARLWDTRVASRAQRTFHGHESDVNTVKFFPDGNRFGTGSDDGSCRLFDIRTGHQLQVYNQPHGDGDIPHVTSMAFSISGRLLFVGYSNGDCYVWDTLLAKVVLNLGSVQNSHEGRISCLGLSADGSALCTGSWDTNLKIWAFGGHRSVV

SEQ ID NO: 9 - DNA, 1600 bp, Nicotiana tabacum
ttcgcggccgccttccctgactcgccactgactcagcctgactcgttctctcctctcctcctcagaaaaaaccctaatttaatcaacgattgttccacaatattgagattttcagaagaattatgtttgaatttccttgaaaatgtcagtgacagagctgaaagagcggcatatggccgctacacagactgtaaatgatctccgtgaaaaacttaagcagaagcgtctccaattactcgacactgatgtttctggatatgcaaggtcgcaaggtaaaactccggtcacctttggcccaacagatctggtttgttgtaggatcctgcaaggacacactggaaaggtatattcactggattggactccagaaaagaatcgtatagtcagtgcatcccaagatggcagattaatagtgtggaatgctctcacaagccagaaaacccatgcaattaagcttccgtgtgcttgggttatgacctgcgccttctctcctagtgggcagtctgttgcctgcggtggccttgacagtgtctgctctatcttcaacttaaattcgccaatcgataaggatgggaaccatcctgtatcaagaatgcttagtgggcataagggttatgtgtcttcctgtcaatatgttccagatgaggatactcacctaataactagttctggtgatcaaacatgtgtcctttgggatataactactggtctaagaacttctgtctttggaggtgagtttcaatccgggcacactgcagatgtacaaagtgtctcaattagttcatcaaaccccagactgtttgtatctgggtcctgtgacacaactgctggactgtgggacacccgagttgctagtcgagctcaacgaacattttatggtcacgagggagatgttaatactgtaaagttctcccctgatggtaatagatttggaactggttcagaggatggaacctgcagattatttgacattaggactggacaccagctgcaagtgtactaccagccgcatggtgatggtgatatccctcatgtgacttccatggcattttctatctcaggccgtcttctctttgtcggatactcaaatggtgattgttatgtgtgggacaccctattagcaaaggtggtcctaaacttgggaggagttcaaaactctcatgaagggcgaataagttgcctgggactgtcagctgatggaagcgccttatgtacaggaagttgggatacaaacctgaagatttgggcttttggagggcacagaagtgtgatctgattgatgaaacacctcattctgttatttaattcctgtcccttttcattctcattttctttcatagctagcctattattcgcgtttcctttggcattgtcataacctgtagatctcttgtattccagttaatatatcaggcagagaaaccaaactgttccatttgcgatcatatgaatctgacaaatattactggatcagcaccagttgtaaagatagcctgtttgttttcaaaattgtctgatggtttcagctgcttctgtaattaaaattctataatagacgcttgaagaatgcaaacaagcttttctttttcgcggccgcgaa

SEQ ID NO: 10 - protein, 377 aa, Nicotiana tabacum
MSVTELKERHMAATQTVNDLREKLKQKRLQLLDTDVSGYARSQGKTPVTFGPTDLVCCRILQGHTGKVYSLDWTPEKNRIVSASQDGRLIVWNALTSQKTHAIKLPCAWVMTCAFSPSGQSVACGGLDSVCSIFNLNSPIDKDGNHPVSRMLSGHKGYVSSCQYVPDEDTHLITSSGDQTCVLWDITTGLRTSVFGGEFQSGHTADVQSVSISSSNPRLFVSGSCDTTAGLWDTRVASRAQRTFYGHEGDVNTVKFSPDGNRFGTGSEDGTCRLFDIRTGHQLQVYYQPHGDGDIPHVTSMAFSISGRLLFVGYSNGDCYVWDTLLAKVVLNLGGVQNSHEGRISCLGLSADGSALCTGSWDTNLKIWAFGGHRSVI

SEQ ID NO: 11 - DNA, 1560 bp, Nicotiana tabacum
gccactgactcagcctgactcgttctctcctctcctcttcagaaaaaaccctaatttaatcaacgattgttccacaatattgagattttcagaagaattatgtttgaatttccttgaaaatgtcagtgacagagctgaaagagcggcatatggccgctacacagactgtaagtgatctccgtgaaaaacttaagcagaagcgtctccaattactcgacactgatgtttctggatatgcaaggtcgcaaggtaaaactccggtcacctttggcccaacagatctggtttgttgtaggatcctgcaaggacacactggaaaggtatattcactggattggactccagaaaagaatcgtatagtcagtgcatcccaagatggcagattaatagtgtggaatgctctcacaagccagaaaacccatgcaattaagcttccgtgtgcttgggttatgacctgcgccttctctcctagtgggcagtctgttgcctgcggtggccttgacagtgtctgctctatctacaacttaaattcgccaatcgataaggatgggaaccatcctgtatcaagaatgcttagtgggcataagggttatgtgtcttcctgtcaatatgttccagatgaggatactcacctaataactagttctggtgatcaaacatgtgtcctttgggatataactactggtctaagaacttctgtctttggaggtgagtttcaatccgggcacactgcagatgtacaaagtgtctcaattagttcatcaaaccccagactgtttgtatctgggtcctgtgacacaactgctcgactgtgggacaaccgagttgctagtcgagctcaacgaacattttatggtcacgagggagatgttaatactgtaaagttcttccctgatggtaatagatttggaactggttcagaggatggaacctgcagattatttgacattaggactggacaccagctgcaagtgtactaccagccgcatggtgatggtgatatccctcatgtgacttccatggcattttctatctcaggccgtcttctctttgtcggatactcaaatggtgattgttatgtgtgggacaccctattagcaaaggtggtcctaaacttgggaggagttcaaaactctcatgaagggcgaataagttgcctgggactgtcagctgatggaagcgccttatgtacaggaagttgggatacaaacctgaagatttgggcttttggagggacagaagtgtgatctgattgatgaaacacctcattctgttatttaattcctgtcccttttcattctcattttctttcatagctagcctattattcgcgtttcctttggcattgtcataacctgtagatctcttgtattccagttaatatatcaggcagagaaaccaaactgttccatttgcgatcatatgaatctgacaaatattactggatcagcaccagttgtaaagatagcctgtttggtccaattcggcacgcgttttttttttttttttttttttttttttttttttttttttttttttttttttttttttttttttttt

SEQ ID NO: 12 - protein, 375 aa, Nicotiana tabacum
MSVTELKERHMAATQTVSDLREKLKQKRLQLLDTDVSGYARSQGKTPVTFGPTDLVCCRILQGHTGKVYSLDWTPEKNRIVSASQDGRLIVWNALTSQKTHAIKLPCAWVMTCAFSPSGQSVACGGLDSVCSIYNLNSPIDKDGNHPVSRMLSGHKGYVSSCQYVPDEDTHLITSSGDQTCVLWDITTGLRTSVFGGEFQSGHTADVQSVSISSSNPRLFVSGSCDTTARLWDNRVASRAQRTFYGHEGDVNTVKFFPDGNRFGTGSEDGTCRLFDIRTGHQLQVYYQPHGDGDIPHVTSMAFSISGRLLFVGYSNGDCYVWDTLLAKVVLNLGGVQNSHEGRISCLGLSADGSALCTGSWDTNLKIWAFGGTEV

SEQ ID NO: 13 - DNA, 1434 bp, Nicotiana tabacum
gaaccctaatttaatcaaccctttttccacgatattgagattttcagaagaattatgtttgaatttccttgaaaatgtcagtgaaagagctgaaagagcggcatatggccgctacacaaactgtaaatgatctccgtgaaaaacttaagcagaagcgtctccaattactcgacactgatgtatctgggtatgcaaggtcgcaaggtaaaactccggtcatctttggcccaacagatctggtttgttgtaggatcctgcaaggacacactggaaaggtatattcactggattggactccagaaaagaatcgtatagtcagtgcatcccaagatggcagattaatagtgtggaatgctctcacaagccagaaaacccatgcaattaagcttccatgtgcttgggttatgacctgcgccttctctcctagtgggcagtctgttgcctgcggtggccttgacagtgtctgctctatcttcaacttaaattcaccgatcgataaggatgggaaccatcctgtatcaagaatgcttagtgggcataaggggtatgtgtcttcctgtcagtatgttccagatgaggatactcacgtaataactagttctggtgatcaaacatgtgtcctttgggatataactactggcttaagaacttctgtctttggaggtgagtttcaatccgggcacaccgcagatgtacaaagtgtctcaattagttcatcaaaccccagactgtttgtgtctgggtcctgtgactcaactgctcgactatgggacacccgagttgctagtcgagctcaacgaacattttatggtcatgagggagatgttaatactgtaaagttcttccctgatggtaatagatttggaactggttcagatgatggaacctgcagattatttgacattaggactggacaccagctgcaagtgtactaccagccgcatggtgatggtgatatccctcatgtgacttccatggcattttctatctcaggccgtcttctctttgtcgggtactcaaatggtgattgttatgtgtgggacaccctattagcaaaggtggtcctaaacttgggagcagttcaaaactctcatgaagggcgaataagttgcctgggactgtcagctgatgggagcgccttatgtacaggaagttgggatacaaacctgaagatttgggcttttggagggcacagaagtgtgatctgaatgatgaaacacctcattctgttatttaattcctgtcccttttcattctcattttctttcatagctagcctattattcgcgtttcctttggcattgtcataacctgtagatctcttgtattccagttaatatatcaggcagagaaaccaaactgttccacttgtgatcatatgtgcgatcatatgaaaatgacaaatattactggatcaaaaaaaaaaaaaaaaaa

SEQ ID NO: 14 - protein, 377 aa, Nicotiana tabacum
MSVKELKERHMAATQTVNDLREKLKQKRLQLLDTDVSGYARSQGKTPVIFGPTDLVCCRILQGHTGKVYSLDWTPEKNRIVSASQDGRLIVWNALTSQKTHAIKLPCAWVMTCAFSPSGQSVACGGLDSVCSIFNLNSPIDKDGNHPVSRMLSGHKGYVSSCQYVPDEDTHVITSSGDQTCVLWDITTGLRTSVFGGEFQSGHTADVQSVSISSSNPRLFVSGSCDSTARLWDTRVASRAQRTFYGHEGDVNTVKFFPDGNRFGTGSDDGTCRLFDIRTGHQLQVYYQPHGDGDIPHVTSMAFSISGRLLFVGYSNGDCYVWDTLLAKVVLNLGAVQNSHEGRISCLGLSADGSALCTGSWDTNLKIWAFGGHRSVI

SEQ ID NO: 15 - DNA, 1430 bp, Nicotiana tabacum
cccctaatttaatcaacgattgttccacaatattgagattttcagaagaattatgtttgaatttccttgaaaatgtcagtgacagagctgaaagagcggcatatggccgctacacagactgtaaatgatctccgtgaaaaacttaagcagaagcgtctccaattactcgacactgatgtttctggatatgcaaggtcgcaaggtaaaactccggtcacctttggcccaacagatctggtttgttgtaggatcctgcaaggacacactggaaaggtatattcactggattggactccagaaaagaatcgtatagtcagtgcatcccaagatggcagattaatagtgtggaatgctctcacaagccagaaaacccatgcaattaagcttccgtgtgcttgggttatgacctgcgccttctctcctagtgggcagtctgttgcctgcggtggccttgacagtgtctgctctatcttcaacttaaattcgccaatcgataaggatgggaaccatcctgtatcaagaatgcttagtgggcataagggttatgtgtcttcctgtcaatatgttccagatgaggatactcacctaataactagttctggtgatcaaacatgtgtcctttgggatataactactggtctaagaacttctgtctttggaggtgagtttcaatccgggcacactgcagatgtacaaagtgtctcaattagttcatcaaaccccagactgtttgtatctgggtcctgtgacacaactgctcgactgtgggacacccgagttgctagtcgagctcaacgaacattttatggtcacgagggagatgttaatactgtaaagttcttccctgatggtaatagatttggaactggttcagaggatggaacctgcagattatttgacattaggactgaacaccagctgcaagtgtactaccagccgcatggtgatggtgatatccctcatgtgacttccatggcattttctatctcaggccgtcttctctttgtcggatactcaaatggtgattgttatgtgtgggacaccctattagcaaaggtggtcctaaacttgggaggagttcaaaactctcatgaagggcgaataagttgcctgggactgtcagctgatggaagcgccttatgtacaggaagttgggatacaaacctgaagatttgggcttttggagggcacagaagtgtgatctgattgatgaaacacctcattctgttatttaattcctgtcccttttcattctcattttctttcatagctagcctattattcgcgtttcctttggcattgtcataacctgtagatctcttgtattccagttaatatatcaggcagagaaaccaaactgttccatttgcgatcatatgaatctgacaaatattactggatcagcaccagttgtaaaaaaaaaaaaaaaa

SEQ ID NO: 16 - protein, 377 aa, Nicotiana tabacum
MSVTELKERHMAATQTVNDLREKLKQKRLQLLDTDVSGYARSQGKTPVTFGPTDLVCCRILQGHTGKVYSLDWTPEKNRIVSASQDGRLIVWNALTSQKTHAIKLPCAWVMTCAFSPSGQSVACGGLDSVCSIFNLNSPIDKDGNHPVSRMLSGHKGYVSSCQYVPDEDTHLITSSGDQTCVLWDITTGLRTSVFGGEFQSGHTADVQSVSISSSNPRLFVSGSCDTTARLWDTRVASRAQRTFYGHEGDVNTVKFFPDGNRFGTGSEDGTCRLFDIRTEHQLQVYYQPHGDGDIPHVTSMAFSISGRLLFVGYSNGDCYVWDTLLAKVVLNLGGVQNSHEGRISCLGLSADGSALCTGSWDTNLKIWAFGGHRSVI

SEQ ID NO: 17 - DNA, 1526 bp, Pisum sativum
gatcattattaggtcaaattcattcacttcaatttccattcacttgaaaaaatgtccgttgcggagctcaaagaacgtcacatagcagcgacggaaacggttaacaatctcagagaacgattgaagcagagacggctttctttgcttgatacagatattgctggatatgctaggtctcaaggtagagctcctgttacttttggtcccactgatattctttgctgtagaacgctccaaggtcataccggaaaggtgtattcattggattggacttcagaaaagaataggattgttagtgcatcccaagatggaagattaatagtgtggaatgctctaacaagccagaaaactcatgctataaagcttccttgtgcatgggtcatgacgtgtgctttctcaccaactggtcaatctgttgcttgtgggggccttgacagtgtttgctctattttcaatcttaattctcccactgatagggatgggaatctaaatgtttcacggatgcttagtggacataaaggttatgtttcatcttgtcagtatgttccaggtgaagacactcacttaatcactggttctggagatcagacatgtgttttatgggatattactactggccttagaacatctgtttttggaggcgagtttcagtctggacatactgcagatgtacttagcatttccattaatggatccaactccaaattgtttgtatctggttcttgcgatgcgactgccagattgtgggacactcgtgtggcaagtcgagcagtgcggacatttcacggccacgagggagatgttaattctgtcaaattctttcctgatggaaatagatttggaactggctcagaggatggaacttgcagattatttgacattaggaccggacaccaacttcaagtatataatcagcaacaccaagacaacgaaatggcacatgtgacgtccattgcattttccatatccggaagacttcttattgctggctatacaaatggtgattgctatgtttgggatactttattggctaaggtggtcttgaatctaggatctcttcaaaactctcatgagggcaggatcacctgtttgggtatgtctgctgatggaagtgctttatgtacaggaagttgggacacaaatttaaagatatgggcatttggagggcataggaaggtgatttgactccattgttagggcttcaccttgttaatgatgcttgtgatattgactttgatccagaattggaaggcaaagtttatctccatgtttataacctttagcagtggaactagtgcagcctttatttatctccatgctcattggttcgtgtgtgtgatttaggtatatatatacctttaaaccaaaacagaggactatttaattttctgtctcctcaatttaactatttgaagtatgtgtttggttcacattggaagaactaaatgtactagtatgtttatagtggttgaatcagatttggatcaggtaagggggtgtttggatccccattgtaaaaaaaaaaa

SEQ ID NO: 18 - protein, 377 aa, Pisum sativum
MSVAELKERHIAATETVNNLRERLKQRRLSLLDTDIAGYARSQGRAPVTFGPTDILCCRTLQGHTGKVYSLDWTSEKNRIVSASQDGRLIVWNALTSQKTHAIKLPCAWVMTCAFSPTGQSVACGGLDSVCSIFNLNSPTDRDGNLNVSRMLSGHKGYVSSCQYVPGEDTHLITGSGDQTCVLWDITTGLRTSVFGGEFQSGHTADVLSISINGSNSKLFVSGSCDATARLWDTRVASRAVRTFHGHEGDVNSVKFFPDGNRFGTGSEDGTCRLFDIRTGHQLQVYNQQHQDNEMAHVTSIAFSISGRLLIAGYTNGDCYVWDTLLAKVVLNLGSLQNSHEGRITCLGMSADGSALCTGSWDTNLKIWAFGGHRKVI

SEQ ID NO: 19 - DNA, 1611 bp, Pisum sativum
ctttcattcactttttctcccctaacaactaaccgtctttgtttctatctgaaaatcaaacaacaataatggaaagtatagttgtagattcatatcattattaggtcaaattcattcacttcaatttccattcacttgacaaaatgtccgttgcggacgtcaaagaacgtcacatagcagcgacggaaacggttaacaatctcagagaacgattgagcagagaccggctttctttgcttgatacagatattgctggatatgctaggtctcaaggtagagctcctgttacttttggtcccactgatattctttgctgtagaacgctccaaggtcataccggaaaggtgtattcattggattggacttcagaaaagaataggattgttagtgcatcccaagatggaagattaatagtgtggaatgctctaacaagccagaaaactcatgctataaagcttccttgtgcatgggtcatgacgtgtgctttctcaccaactggtcaatctgttgcttgtgggggccttgacagtgtttgctctattttcaatcttaattctccactcgatagggatgggaatctaaatgtttcacggatgcttagtggacataaaggttatgtttcatcttgtcagtatgttccaggtgaagacactcacttaatcactggttctggagatcagacatgtgttttatgggatattactactggccttagaacatctgtctttttaggcgagtttcagtctggacatactgcagatgtacttagcatttccattaatggatccaactccaaattgtttgtatctggttcttgcgatgcgactgccagattgtgggacactcgtgtggcaagtcgagcagtgcggacatttcacggccacgagggagatgttaattctgtcaaattctttcctgatggaaatagatttggaactggctcagaggatggaacttgcagattatttgacattaggaccggacaccaacttcaagtatataatcagcaacaccaagacaacgaaatggcacatgtgacgtccattgcattttccatatccggaagacttcttattgctggctatacaaatggtgattgctatgtttgggatactttattggctaaggtggtcttgaatctaggatctcttcaaaactctcatgagggcaggatcacctgtttgggtatgtctgctgatggaagcgctttatgtacaggaagttgggacacaaatttaaagatatgggcatttggagggcataggaaggtgatttgactccattgttagggcttcaccttgtaatgatgcttgtgatattgactttgatccagaattggaaggcaaagtttatctccatgtatacttagcagtgactagtgcagcttattatctcatgctcattggttcgtgtgtgtgatttaggtatatatatacctttaaaccaaaacagaggacttataattttgtgtctcctcaatttaactattgaagtagtgtttggttcacattggaagaactaaatgtactagtatgtttatagtggttgaatcagatttggctcaggtaagggggtgtttggatccccattgtaaaaaaaaaaaaaaaaaaa

SEQ ID NO: 20 - protein, 377 aa, Pisum sativum
MSVADVKERHIAATETVNNLRERLSRDRLSLLDTDIAGYARSQGRAPVTFGPTDILCCRTLQGHTGKVYSLDWTSEKNRIVSASQDGRLIVWNALTSQKTHAIKLPCAWVMTCAFSPTGQSVACGGLDSVCSIFNLNSPLDRDGNLNVSRMLSGHKGYVSSCQYVPGEDTHLITGSGDQTCVLWDITTGLRTSVFLGEFQSGHTADVLSISINGSNSKLFVSGSCDATARLWDTRVASRAVRTFHGHEGDVNSVKFFPDGNRFGTGSEDGTCRLFDIRTGHQLQVYNQQHQDNEMAHVTSIAFSISGRLLIAGYTNGDCYVWDTLLAKVVLNLGSLQNSHEGRITCLGMSADGSALCTGSWDTNLKIWAFGGHRKVI

SEQ ID NO: 21 - DNA, 1470 bp, Avena fatua
atggcgtctgttgctgaacttaaagagaggcacgcggcggcgacggcctcggtgaactctctgcgagagaggctccgtcagcggcggcagacgctcctcgacactgacgtggagaaatactccaaggcgcaggggcggacggcggtgagcttcaaccagacggatctggtgtgctgccgcacgctgcagggccacagcggaaaggtatattctctggattggactcctgaaaagaactggatagtcagcgcctcacaagatggaagactaattgtatggaatgctttaacgagtcaaaaaacacatgccataaagctacactgtccatgggtgataacatgtgcttttgcacccaatggtcaatctgttgcctgtggtggtctgaatagtgcatgctctatatttaatcttaattcccaagtggacagaaatggaaacatgccagtatcaaaattacttactggaccaaagggctatgttttgtcctgtcagtatgtccctgatcaggaaacccgcatgattacaggctcaggtgacccaacgtgtgtcctatgggatgttactactggccaaagaatatccatctttggaggtgaattcccatcaggccatacagctgacgtgttaagtctgtccatcaactcgttaaacacaaatatgtttgtctcgggttcatgtgatacaactgtaaggctatgggatctcagaatagcaagccgggcagtccgaacatatcatggacatgaaggcgatattaacagtgtcaagtttttccctgatggtcataggtttggtactggttcagatgatggtacatgcagattatttgacatgagaatcaggcatcaacttcaagtgtacagtcgggagccagatagaaatgataatgagctccctagcgttacatctattgcattctccatatcaggaaggcttctctttgctggttactctaatggtgactgttatgcgtgggacacgcttctcgccgaggtagtgctcaatttgggaactctccaaaactcccacgaaggtcgtataagctgccttgggttgtcatctgatgggagtgcattgtgtacaggaagttgggacaaaaatttgaagatttgggccttcagtggacaccgcaaaatagtctgaagccgcccagcggtcttctctccatgttgtatgttcctcctcctcgcttgttgaagaatggtggccaactcaacaggttcctgaagatgaagttgttggttttgtagcatagaaatcttcctgtatcataccttatgtccagtggaaaaatacagtttatcggcggagactgtgccgtgatgttcttgtacctggtcaagtcagcgtactgttaatagagagttattactataaatcagcacccatgtgatctttttctgttctttctatgtgcaattatttcagctgtagaaaagcactaccttgtgatgtcttaaaaaaaaaaaa

SEQ ID NO: 22 - protein, 380 aa, Avena fatua
MASVAELKERHAAATASVNSLRERLRQRRQTLLDTDVEKYSKAQGRTAVSFNQTDLVCCRTLQGHSGKVYSLDWTPEKNWIVSASQDGRLIVWNALTSQKTHAIKLHCPWVITCAFAPNGQSVACGGLNSACSIFNLNSQVDRNGNMPVSKLLTGPKGYVLSCQYVPDQETRMITGSGDPTCVLWDVTTGQRISIFGGEFPSGHTADVLSLSINSLNTNMFVSGSCDTTVRLWDLRIASRAVRTYHGHEGDINSVKFFPDGHRFGTGSDDGTCRLFDMRIRHQLQVYSREPDRNDNELPSVTSIAFSISGRLLFAGYSNGDCYAWDTLLAEVVLNLGTLQNSHEGRISCLGLSSDGSALCTGSWDKNLKIWAFSGHRKIV

SEQ ID NO: 23 - DNA, 1664 bp, Oryza sativa
caccccatttcggggtccgtttctcgccgccgccgccgtggtagtatcctcctcctcctcgcgagctccggaaagctccagccgaggccattgcgttcctcgcctccatgcccggatcccagtagatcccccctccctcaaccagcgcgaggtcgcggggggcgtgcgggcggcggcggcatggcgtccgtggcggagctcaaggagaagcacgcggcggccacggcgtcggtgaactccctgcgggagcggctccgtcagaggcggcagatgctgctcgacaccgacgtggagaggtactcgaggacgcaggggcggacgccggtgagcttcaacccgacggatctggtgtgctgccgcacgcttcaaggccacagcggaaaggtatattctctggattggacccctgaaaagaattggatagtcagtgcctcacaagatggaaggctaattgtatggaatgcattaacaagtcaaaaaacacatgccataaagttacattgcccatgggtgatgacatgtgcatttgcacccaatggccaatctgttgcctgtggtggtcttgacagcgcatgctctatcttcaatcttaactcacaagcagacagagatgggaatataccagtatcaagaatacttaccggacacaaaggctatgtttcatcctgtcagtatgtcccagatcaggaaacccgcctaattactagctctggtgatcaaacatgtgtcctgtgggatgttactactggccaaaggatatcaatatttggcggtgaattcccatcagggcatacggcagatgttttgagcttgtccataaactcatcaaattcgaatatgtttgtttcgggttcatgtgatgcaactgtaaggctgtgggatatcagaattgcaagccgggcagttagaacatatcatggtcatgagggtgacattaacagtgtcaagtttttccctgatggccagaggtttggtactggttcagatgatggaacgtgtagattatttgacgtgagaacagggcaccaacttcaagtatacagtcgggaacctgatagaaatgataatgaactcccaactgttacatctattgcattttcgatatcaggaaggcttctttttgctggatactccaatggtgactgttatgtgtgggacacacttctcgctgaggtggtacttaatttgggaaacctccaaaactctcatgaggggcgtataagctgccttggtctttcttctgatgggagtgcattgtgtacaggaagttgggacaagaatttgaagatttgggccttcagcggacaccggaaaatagtttgaaggacagttttcttcctgtgttgttgtaagttccttgtgttaagaattacgaccaactcgatgggctatggaaatcagtttgttggtcttgtagcatagaatcaggcaatcagctgtatcatatcctaatgtccagtggaaaaatgcaatctgttgtcttggcaagacgtgtgctctgactcaccgtgttaagttgatgtagtgtttatattaccagaaagcatcatccattcggatcggtctcttcttgttgtataacttctttttgtagtagaaagctaccaacttttgcatttgtatttcacaactgattatgaatttgtccctttccaagtcagccc

SEQ ID NO: 24 - protein, 380 aa, Oryza sativa
MASVAELKEKHAAATASVNSLRERLRQRRQMLLDTDVERYSRTQGRTPVSFNPTDLVCCRTLQGHSGKVYSLDWTPEKNWIVSASQDGRLIVWNALTSQKTHAIKLHCPWVMTCAFAPNGQSVACGGLDSACSIFNLNSQADRDGNIPVSRILTGHKGYVSSCQYVPDQETRLITSSGDQTCVLWDVTTGQRISIFGGEFPSGHTADVLSLSINSSNSNMFVSGSCDATVRLWDIRIASRAVRTYHGHEGDINSVKFFPDGQRFGTGSDDGTCRLFDVRTGHQLQVYSREPDRNDNELPTVTSIAFSISGRLLFAGYSNGDCYVWDTLLAEVVLNLGNLQNSHEGRISCLGLSSDGSALCTGSWDKNLKIWAFSGHRKIV

SEQ ID NO: 25 - DNA, 1671 bp, Zea mays
gctgtcggcgccgccgcctgtcctaatctcctctgagtccagcggccacctcctccaccgggagctccccgtaccataaccgcagtccgcagccattggaatttccgcttcatgcgtggatcctcgtagaccccgacccgcgtgcactcaatccctaggcggcggcctccggcgcgaggctagcgggcggcacccatggcgtccgtggcggagctcaaggagaagcacgccgcagctacggcgtcggtgaactccctgcgcgagcgcctccgccagcgccgggagacgctcctcgacaccgacgtggcgaggtactccaagtcgcaggggagggtgccggtgagcttcaaccctacggatctggtctgctgccgcacgctgcagggccatagcggaaaggtatattctctggattggacccctgaaaagaattggatagtcagtgcctctcaagatggaaggttaattgtgtggaatgcattgacaagccagaaaacacatgccataaagctgcattgcccatgggttatggcgtgtgcttttgcacccaatggccaatctgtcgcctgtggtggtcttgatagtgcgtgctctattttcaatctcaattctcaagcagacagagatgggaacatgccagtatcaagaattcttactggacacaagggctatgtctcatcatgtcaatatgtcccagatcaggaaacacgtcttattactagttcaggtgatcaaacatgtgttctttgggatgttactactggacagaggatatcaatatttggtggtgaattcccatcagggcatacagctgatgttcaaagtgtgtccatcaactcatcaaatacaaatatgtttgtctctggctcatgtgatacaactgtgaggctgtgggatatcagaattgcaagtcgagctgttcgaacctaccatggacatgaggatgatgttaacagtgtgaagtttttccctgatggccataggtttggtactggctcagatgatggcacatgtagattatttgatatgagaacagggcatcaacttcaggtgtacagtagggagcctgatagaaatagtaatgaactacctactgttacatctattgcattttcaatatcaggaaggctactttttgctggttactccaatggtgactgttatgtgtgggacacacttctcgccgaggtggtacttaatttgggaaacctgcaaaactcccatgatggccgtataagttgcctcgggatgtcatctgatgggagtgcattgtgtacaggaagctgggacaaaaatttgaagatttgggccttcagtggacaccggaagatagtttgaaggccaacttttctcccccatgttgtatgttccttgttgccccttaacaacggacagtggtgattggtgaccaactcgacttgttcctgggaatccctttgttgttttgtaagctctgttcgcgctatgtttaatggaaaaatgtgcaatttgtcagtgtcacggcgctacatcttgttgagttggtaactgtttatactgttattacgagaatatcagtaacgtgtgatctgcccttttctttgtacaaccgtttgatcttttcaggttttgtgaagtagcatgtgtttccttaatcaatttatcatatcagtttgtccatttgctgaatta

SEQ ID NO: 26 - protein, 380 aa, Zea mays
MASVAELKEKHAAATASVNSLRERLRQRRETLLDTDVARYSKSQGRVPVSFNPTDLVCCRTLQGHSGKVYSLDWTPEKNWIVSASQDGRLIVWNALTSQKTHAIKLHCPWVMACAFAPNGQSVACGGLDSACSIFNLNSQADRDGNMPVSRILTGHKGYVSSCQYVPDQETRLITSSGDQTCVLWDVTTGQRISIFGGEFPSGHTADVQSVSINSSNTNMFVSGSCDTTVRLWDIRIASRAVRTYHGHEDDVNSVKFFPDGHRFGTGSDDGTCRLFDMRTGHQLQVYSREPDRNSNELPTVTSIAFSISGRLLFAGYSNGDCYVWDTLLAEVVLNLGNLQNSHDGRISCLGMSSDGSALCTGSWDKNLKIWAFSGHRKIV

SEQ ID NO: 27 - DNA, 1453 bp, Solanum tuberosum
atgggctcgttgtgcagcagcagaaacaaacactacagtcaagccgatgatgaggaaaatactcagactgcagagatagaaagacggattgaacaagaaacaaaggcagacaagcatattcagaaacttcttctacttggtgccggagattcggggaagtctacgatttttaaacagataaaactcttgttccaaactggctttgatgaagcagagctaaagaactacatccctgtgattcatgccaatgcttatcagacaataaaaatattacatgatggatcaaaggaattagctcaaaatgaattagaggcctcaaagtatcttctatcagctgaaaataaggagatcggcgagaagctttcagaaattggaggcaggttggattatcctcgcctgactaaggatctggtgcaggatattgaagctctttggaaagatcctgctattcaagaaactctgttacgtggtaatgagcttcaggttccagattgtgcccattatttcatggaaaacttggagagattttctgatatacattatattccaacaaaggaggatgttctttttgcccggattcgaacgacaggtgtcgttgaaatacagttcagtccagttggagagaacaaaaaaagtggagaagtgtataggctttttgatgttggaggtcagagaaatgagagaagaaagtggattcatctatttgaaggtgtaacagcagttatattttgtgccgctattagtgagtatgatcaaactctatttgaggatgaaagaaagaaccgaatgatggagaccaaggaactctttgagtgggtcttaaagcaaccatgttttgagaaaacttcctgcatgctgtttctcaacaaatttgatatatttgagcagaaggttctgaaagttcccctcaacacttgtgagtggtttaaagattaccagtcagtttcaacaggaaaacaagagattgagcatgcttatgagtttgtaaagaaaaaatttgaggagtcatatttccaatgcactgcaccagatcgtgtggaccgtgtgtttaagatctatagaaccacagcccttgatcagaagcttgtaaagaagacgttcaaactggtagacgagaccctgagaaggagaaacctcttcgaagcaggtttattatgaaattctttaaattttcaaaaaaaaaaaaaacagaaatgttcatacctttgaaagatgcatacaagttttgaacagtgaggttcaaatacagaaaaaacaggctatggtggggggtatcatatcagattcaacaatttaagttttgtccatgttaggtctctaagcacatatttctttctatatcccggtattgttatgttctacttacaaaacagattggatcaaaacaaaaattgatattctattgatgttcattttgttgaatgttgtaacattctcacagcgcgaagttgta

SEQ ID NO: 28 - protein, 385 aa, Solanum tuberosum
MGSLCSSRNKHYSQADDEENTQTAEIERRIEQETKADKHIQKLLLLGAGDSGKSTIFKQIKLLFQTGFDEAELKNYIPVIHANAYQTIKILHDGSKELAQNELEASKYLLSAENKEIGEKLSEIGGRLDYPRLTKDLVQDIEALWKDPAIQETLLRGNELQVPDCAHYFMENLERFSDIHYIPTKEDVLFARIRTTGVVEIQFSPVGENKKSGEVYRLFDVGGQRNERRKWIHLFEGVTAVIFCAAISEYDQTLFEDERKNRMMETKELFEWVLKQPCFEKTSCMLFLNKFDIFEQKVLKVPLNTCEWFKDYQSVSTGKQEIEHAYEFVKKKFEESYFQCTAPDRVDRVFKIYRTTALDQKLVKKTFKLVDETLRRRNLFEAGLL

SEQ ID NO: 29 - DNA, 1276 bp, Solanum tuberosum
ctggcatacatggacatcataaaggagctgtaacaacagatttgagatccctagtttgactatcacgcaggcctatgctgtcggtggttttagaaaacatgggctcgttgtgcagcagaaacaaacactacagtcaagccgatgatgaggaaaatactcagactgcagagatagaaagacggattgaacaagaaacaaaggccgacaagcatattcagaaacttcttctacttggtgccggagattcggggaagtctacgatttttaaacagataaaactcttgttccaaactggctttgatgaagcagagctaaagaactacatccctgtgattcatgccaatgtttatcagacaataaaaatattacatgatggatcaaaggaattagctcaaaatgaattagaggcctcaaagtatcttctatcagctgaaaataaggagatcggtgagaagctttcagaaattggaggcaggttggattatcctcgcctgactaaggatctggtgcaggatattgaagctctttggaaagatcctgctattcaagaaactctgttacgtggtaatgagcttcaggttccagattgtgcccattatttcatggaaaacttggagagattttctgatatacattatattccaacaaaggaggatgttctttttgcccggattcgaacgacaggtgtcgttgaaatacagttcagtccagttggagagaacaaaaaaagtggagaagtgtataggctttttgatgttggaggtcagagaaatgagagaagaaagtggattcatctatttgaaggtgtaacagcagttatattttgtgccgctattagtgagtatgatcaaactctatttgaggatgaaagaaagaaccgaatgatggagaccaaggaactctttgagtgggtcttaaagcaaccatgttttgagaaaacttccttcatgctgtttctcaacaaatttgatatatttgagcagaaggttctgaaagtgcccctcaacacttgtgagtggtttaaggattaccagtcagtttcaacaggaaaacaagagattgagcatgcttatgagtttgtaaagaaaaaatttgaggagtcatatttccaatgcactgcaccagattgtgtggaccgtgtgtttaagatctatagaaccacagcccttgatcagaagcttgtaaagaagacgttcaaactggtagacgagaccctgagaaggagaaacctattcgaagcaggtttattatgaaattctttaaattttcaaaaaaa

SEQ ID NO: 30 - protein, 392 aa, Solanum tuberosum
MLSVVLENMGSLCSRNKHYSQADDEENTQTAEIERRIEQETKADKHIQKLLLLGAGDSGKSTIFKQIKLLFQTGFDEAELKNYIPVIHANVYQTIKILHDGSKELAQNELEASKYLLSAENKEIGEKLSEIGGRLDYPRLTKDLVQDIEALWKDPAIQETLLRGNELQVPDCAHYFMENLERFSDIHYIPTKEDVLFARIRTTGVVEIQFSPVGENKKSGEVYRLFDVGGQRNERRKWIHLFEGVTAVIFCAAISEYDQTLFEDERKNRMMETKELFEWVLKQPCFEKTSFMLFLNKFDIFEQKVLKVPLNTCEWFKDYQSVSTGKQEIEHAYEFVKKKFEESYFQCTAPDCVDRVFKIYRTTALDQKLVKKTFKLVDETLRRRNLFEAGLL

SEQ ID NO: 31 - DNA, 1558 bp, Solanum tuberosum
tgttttcgaaaacatgggctcgttgtgcagcagaaacaaacactacagtcaagccgatgatgaggaaaatactcagactgcagagatagaaagacggattgaacaagaaacaaaggcagacaagcatattcagaaacttcttctacttggtgccggagattcggggaagtctacgatttttaaacagataaaactcttgttccaaactggctttgatgaagcagagctaaagaactacatccctgtgattcatgccaatgtttatcagacaataaaaatattacatgatggatcaaaggaattagctcaaaatgaattagaggcctcaaagtatcttctatcagctgaaaataaggagatcggcgagaagctttcagaaattggaggcaggttggattatcctcgcctgactaaggatctggtgcaggatattgaagctctttggaaagatcctgctattcaagaaactctgttacgtggtaatgagcttcaggttccagattgtgcccattatttcatggaaaacttggagagattttctgatatacattatattccaacaaaggaggatgttctttttgcccggattcgaacgacaggtgtcgttgaaatacagttcagtccagttggagagaacaaaaaaagtggagaagtgtataggctttttgatgttggaggtcagagaaatgagagaagaaagtggattcatctatttgaaggtgtaacagcagttatattttgtgccgctattagtgagtatgatcaaactctatttgaggatgaaagaaagaaccgaatgatggagaccaaggaactctttgagtgggtcttaaagcaaccatgttttgagaaaacttccttcatgctgtttctcaacaaatttgatatatttgagcagaaggttctgaaagttcccctcaacacttgtgagtggtttaaagattaccagtcagtttcaacaggaaaacaagagattgagcatgcttatgagtttgtaaagaaaaaatttgaggagtcatatttccaatgcactgcaccagatcgtgtggaccgtgtgtttaagatctatagaaccacagcccttgatcagaagcttgtaaagaagacgttcaaactggtagacgagaccctgagaaggagaaacctcttcgaagcaggtttattatgaaattctttaaatttttcaaaaaaaaaaaaacagaaatgttcatacctttgaaagatgcatacaagttttgaacagtgaggttcaaatacagaaaaaacaggctatggtggggggtatcatatcagattcaacaatttaagttttgtccatgttaggtctctaagcacatatttctttctatatcccggtattgttatgttctacttacaaaacagatggatcaaaacaaaaattgatattctattgatgttcattttgttgaatgttgtaacattctcacagcgcgaagttgtacatgcattgttggttaacctttttctatctcgtgctttatttagtttatcgtttagctcttacccagcttaagctattaaaaaaaaaaaaaaaaaa

SEQ ID NO: 32 - protein, 384 aa, Solanum tuberosum
MGSLCSRNKHYSQADDEENTQTAEIERRIEQETKADKHIQKLLLLGAGDSGKSTIFKQIKLLFQTGFDEAELKNYIPVIHANVYQTIKILHDGSKELAQNELEASKYLLSAENKEIGEKLSEIGGRLDYPRLTKDLVQDIEALWKDPAIQETLLRGNELQVPDCAHYFMENLERFSDIHYIPTKEDVLFARIRTTGVVEIQFSPVGENKKSGEVYRLFDVGGQRNERRKWIHLFEGVTAVIFCAAISEYDQTLFEDERKNRMMETKELFEWVLKQPCFEKTSFMLFLNKFDIFEQKVLKVPLNTCEWFKDYQSVSTGKQEIEHAYEFVKKKFEESYFQCTAPDRVDRVFKIYRTTALDQKLVKKTFKLVDETLRRRNLFEAGLL

SEQ ID NO: 33 - DNA, 1461 bp, Oryza sativa
gacgtcaacgtgcttcctggaaagagagaggctcaggcatgagagcatacctctaaaataatgtccgtgcttacctgtgtgcttgataacatgggctcatcctgtagcagatctcattctttaagtgaggctgaaacaaccaaaaatgcaaaatctgcagacattgacaggcgaattttgcaagagacaaaagcagagcaacacatccacaagctcttacttcttggtgcgggagaatcagggaagtctacgatatttaaacagattaagctccttttccaaactggctttgatgaggcagaacttaggagctacacatcagttatccatgcaaacgtctatcagacaattaaaatactatatgaaggagcaaaagaactctcacaagtggaatcagattcctcaaagtatgttatatccccagataaccaggaaattggagaaaaactatcagatattgatggcaggttggattatccactgctgaacaaagaacttgtactcgatgtaaaaaggttatggcaagacccagccattcaggaaacttacttacgtggaagtattctgcaacttcctgattgtgcacaatacttcatggaaaatttggatcgattagctgaagcaggttatgtgccaacaaaggaggatgtgctttatgcaagagtacggacaaatggtgttgtacaaatacaatttagtcctgttggagaaaacaaaagaggtggagaggtatataggttgtatgatgtaggaggccagaggaatgagaggagaaagtggattcatctttttgaaggtgttaatgcggtaatcttttgtgctgccattagcgaatatgatcagatgctatttgaagatgagacaaaaaacagaatgatggagaccaaggaactctttgactgggttttaaagcaaagatgttttgagaaaacatcattcattctgtttctcaacaaatttgatatattcgagaagaaaatacaaaaggttcctttaagtgtgtgcgagtggtttaaagactaccagcctattgcacctgggaaacaggaggttgaacatgcatatgagtttgtcaagaagaagtttgaagagctctacttccagagcagcaagcctgaccgtgtggaccgcgtcttcaaaatctacagaactacggccctagaccagaaacttgtaaagaagacattcaagttgattgatgagagcatgagacgctccagggaaggaacttgattcagagctaagactaggttgtaagtcacacagggaaggtaattaggacggcgagaggaacaaagtttcacactgtcacagctttatctgttgtaattcttttacacgtggaccattgattgatcttttggttcttactgtgggctgttcaggtctgtaccctattttttgttctctagttagccattgtgcaaattttccttgaatcagattctctacctgttgtct

SEQ ID NO: 34 - protein, 380 aa, Oryza sativa
MGSSCSRSHSLSEAETTKNAKSADIDRRILQETKAEQHIHKLLLLGAGESGKSTIFKQIKLLFQTGFDEAELRSYTSVIHANVYQTIKILYEGAKELSQVESDSSKYVISPDNQEIGEKLSDIDGRLDYPLLNKELVLDVKRLWQDPAIQETYLRGSILQLPDCAQYFMENLDRLAEAGYVPTKEDVLYARVRTNGVVQIQFSPVGENKRGGEVYRLYDVGGQRNERRKWIHLFEGVNAVIFCAAISEYDQMLFEDETKNRMMETKELFDWVLKQRCFEKTSFILFLNKFDIFEKKIQKVPLSVCEWFKDYQPIAPGKQEVEHAYEFVKKKFEELYFQSSKPDRVDRVFKIYRTTALDQKLVKKTFKLIDESMRRSREGT

SEQ ID NO: 35 - DNA, 1537 bp, Oryza sativa
ggatcctgagatctagacgtgaacgtgcttggtgggaagagagaggctcaggcatgagagcatgcctctaaaataatgtccgtgcttacctgtgtgtttgataacatgggctcatcctgtagcagatctcattctttaagtgaggctgaaacaacaaaaaatgcaaaatctgcagacattgacaggcgaattttgcaagagacaaaagcagagcaacacatccacaagctcttacttcttggtgcgggagaatcagggaagtctacgatatttaaacagattaagctccttttccaaactggctttgatgaggcagaacttaggagctacacatcagttatccatgcaaacgtctatcagacaattaaaatactatatgaaggagcaaaagaactctcacaagtggaatcagattcctcaaagtatgttatatccccagataaccaggaaattggagaaaaactatcagatattgatggcaggttggattatccactgctgaacaaagaacttgtactcgatgtaaaaaggttatggcaagacccagccattcaggaaacttacttacgtggaagtattctgcaacttcctgattgtgcacaatacttcatggaaaatttggttcgattagccgaagcaggttatgtgccaacaaaggaggatgtgctttatgcaagagtacggacaaatggtgttgtacaaatacaatttagtcctgttggagaaaacaaaagaggtggagaggtatataggttgtatgatgtaggaggccagaggaatgagaggagaaagtggattcatctttttgaaggtgttaatgcggtaatcttttgtgctgccattagcgaatatgatcagatgctatttgaagatgagacaaaaaacagaatgatggagaccaaggaactctttgactgggttttaaagcaaagatgttttgagaaaacatcattcattctgtttctcaacaaatttgatatatgcgagaagaaaatacaaaaggttcctttaagtgtgtgcgagtggtttaaagactaccagcctattgcacctgggaaacaggaggttgaacatgcatatgagtttgtcaagaagaagtttgaagagctctacttccagagcagcaagcctgaccgtgtggaccgcgtcttcaaaatctacagaactacggccctagaccagaaacttgtaaagaagacattcaagttgattgatgagagcatgagacgctccagggaaggaacttgattcagagctaagactaggttgtaagtcacacagggaaggtaattaggacggcgagaggaacaaagtttcacactgtcacagctttatctgttgtaattcttttacacgtggaccattgattgaccttttggttcttactgtgggctgttcaggtctgtaccctattttttgttctctagttagccattgtgcaaattttccttgaatcagattctctacctgttgtctatgtgtgttatcttggtctgttaatttgcatagcccacttgttcattaaaaaaaaaaaaaa

SEQ ID NO: 36 - protein, 380 aa, Oryza sativa
MGSSCSRSHSLSEAETTKNAKSADIDRRILQETKAEQHIHKLLLLGAGESGKSTIFKQIKLLFQTGFDEAELRSYTSVIHANVYQTIKILYEGAKELSQVESDSSKYVISPDNQEIGEKLSDIDGRLDYPLLNKELVLDVKRLWQDPAIQETYLRGSILQLPDCAQYFMENLVRLAEAGYVPTKEDVLYARVRTNGVVQIQFSPVGENKRGGEVYRLYDVGGQRNERRKWIHLFEGVNAVIFCAAISEYDQMLFEDETKNRMMETKELFDWVLKQRCFEKTSFILFLNKFDICEKKIQKVPLSVCEWFKDYQPIAPGKQEVEHAYEFVKKKFEELYFQSSKPDRVDRVFKIYRTTALDQKLVKKTFKLIDESMRRSREGT

SEQ ID NO: 37 - DNA, 7360 bp, Nicotiana tomentosiformis
aatcaagccgatgatgaggaaaatactcaggtatgagttttacgaaatatatttggaatttttggtaattgtccgtagtgctgtctttaagctaggagagcaaagaatctattttcatcttgcttaatgcataaccaccagtgtccttactagatcatgtgtctacagactgcagatatagaaagacgtatcgagcaagaaacaaaggcggacaagcatattcagaaacttcttctacttggtaaatcagaaaaattagtcttcaattgtcatatgatctagttatttctcactgtttgcttttcctttttatgttcaccatatcagtgtagtgattcacgatttctatagacatctttcaccttggacttgttaaatttgaacttttcttttcctcgaagcttactgttgtgtgttttgcgtatgattaaacttactgctgtgtgttttttggtctatgttctgtttgactgtgtttgctgaatgctgatctttctgttgtttcaaggtgccggagattcggggaagtccactatttttaagcaggcaagagatcttatgattaatattttacgattcattattctctaactgttcatgttaatgtatttcttttaactccagtgtcctctatttatttttgccatgcagataaaacttttgtttcaaactggctttgatgaagcagagctaaagaactatatccctgtcattcatgccaacgtctatcagacaataaaagtacggaatacttgaaagggtgtgttggttatttctctttttgcaaaatagccgctgcttgttagagacgtgcatatatagaaatatcattcacatgctttataacgaggtttgattactaatgtcacggaaactgacattcttacatggtgggtgtttggtgacataacaggtattacatgatgggtcgaaggaattagcacaaagtgaattagaggcctcaaagtatcttctatcagccgaaaataaggtatgtgggctatcatcttgaagtcatttagacaagggaaacccttgaaattgaggacatgtgattgcttgtcttgcttagctgcatgaacataataacgtatttctttgcaccaggatatcggcgagaagctttcagaaattggaggtaggttggattatcctcacctgactaaggatctggtgcaggatattgaagctctttggaaagatcctgctattcaagtaatcttgcttcgcttaagccctttgatgactttatttcagtgtcaagtacttttttaaggcttgatgatttggatttgtttttacttgtataattaaatgattattgataattgaaacaatcaattcattgaagtcactcaatcaactatgcctcaatccgtaactagtattctagtggggttttcgcgatgatcttctatatattctgctctattggggccatttcacatttcattccaataccactttttctttggctgtaagtcaagttcagtagttctacaaaactagaggttttttaaatctcactcttattcctaatcctaatgttgaaaaccagcaacaactgatccgtattcctatataattggagttgtctatttggataatttgtttccatcatgttctagtcttaactcagttcacaatattcctagatatcatagctctttttagacaagcaccctagggtgtgacctagtggtcaataaagtgggtgcaaagctgggatcaaattccgccggagacaaaaaacagtaggtgatctcttcccatttgcataagccatggtggatagagttactcaatacctatgctggtgggaggtatcgggaactcggtggtataatcgaggtgagcgcaagttggtccagacaccaccgttataaaagaaaatctttttagataacttctctccgtgtgattctagaattaccttgtcccttttttagcaccttcaatcactaaagtcatacctacatattggtgaatttgaagggatgacttagtaatctcaccgtctcacattttattctgtatgtatgttacttgcaccttctctgcctaagcatgggtgggcagaattacctagtacctattttgatgggcggtaacaagtatcggtgaaatagttgaggtgcaagtaaagtggcccaaacgccaccttcataaaaaatgtatgttacttgcactttctgtctgaggtgatcattttaattttgtctacatttgtaagattacaaattcgttataactttcgcatttctaagacactcatcttgaggatacgttgggccttaaccttcattccctcattaccggtctttgcctttcactttattagatatgttcctatcacatagttttcttgtagtacgcctccaccacaatcatctgatctaactctatgtgctacatctttatctgtagtgccattctcctagaagattgagcctatatatttgaataatttgtattaaagcaccacaatcgttcactttaccttcattcttcttgtgtgggctacactggcaatgcatataatactgtcttacttttatttatcccaaaatccttgttctctagatcccctctccatagttgtagcctttagttttattgaagtacatcactttagaatttgattttttcataaaatggccaatcagagattctgattaatcaagctgatggtgaaaaatccgaactacgcaaatccataattttctatgagtatcccagaaaagattaacttttcattactctgatagagttttctttctttcttgtaggaaactatattacgcggtaatgagctccaggttccagattgtgcccattatttcatggaaaacttgcagagattttcggatataaattatgtcccatcaaaggtaagaaatagagactggatcatgtatgttttttgtttctgtattccgattatattaactgaaatatatggaactagaatgctattgaaataaatagaatataggaagtcttgagacactaagttagtatgttttacagttctaccagtagaacagaagattaacaacacattgttcattggttgtataccaactctttaatcaatgaggtattttccaattaagagttcgctttcattgcaataataaaaatctgttctacttttcatattgaatttacgaagaaatcaaagccatctgtactttaacccaatgataaaactatcctctacatgatgttgaattcacagttgttgatagcagttttggctcagatgcattattaaaactattttacccttaggattttggtcctaggggtgtctatggcaaagctatgcatgccaatgtgtaaatgtgtacacacattataagaatcagacctgagaaatgagagacaagtaggagatggtaagctaaacaagtaaatgtgtaaatgacaaatattttttcattttacatgctcttaatccggtggttcttatacatgtattgttattagttatagaataaatttaatacccaattgttaatacaaataattactagattgtttttaaattgttctggaatagaggcggaactatgaataatggagcattatcatataacttaagtgatacaagatagggttgcagcacaattaccaatccctatttaggccataaccaagattatttccaaattatcaaaacgacccttacgtgttcctaaacctacggcttgtttcaagcatccttatccttaaatctattatactgttcatagaactctagtttctaaccatatattatatcaacattggctttttgtggtgatgcttctcgaaagtgctataattgatcgcgcttttatttattaattaatcctcattttaaatgatatggaatatttttagattgttgggtctacgtacctttaaataacctgctttgttttgctttgttatattttttctttttgaagagataatgttcctcttatcattttgtcaattttaaggggtggtgggttcctttctgtccccttcatccattgctcatcgttttccaagctatctctttcgttcattttcttttttcttggtgtaactgagttggtgagtttttgcctttctattagaagtgatgatgagtatttaaactgtaaagaattaagtaatgatagtttacaatgaatacgatgccgcacatttgccttaaatttataagtgggaatctggaattttcaactttggatataccagaaaaagggccacatataatgggttatgtttttatagtttaagtggtatactgtttaaccaaaaaataagatctttgttcgaagcctatttttagaagaactcggattactgataaccaaataatttcttacttcctcagtaactcacttaaaaggaaataaagttgacaaaaagtaactaaaaagaaagaataattgattgcactgaataagcaagagaaagtgaatatatttcgatagtactcaaaacgtgtctttacagatgagattctcccccttatataggatcttgacatgcatagctttgccaggacgggccaaacccgttttctttctgatctcattttaacccacctgtttgacacccctagtgttggtcaaatatttcaaaattcaaactgcaattttctagagtcaaatacgatgtcgagcacccaagatacttgctattacacagtacagttgcccttgcttatcgcttatgcctgtatctaattttctattacactagtggtgttctttctataggcagtgtaagtgtaggaagtgcaagtgttgcctgtctattgctggtaaagaaaagtgtggtccatcaatcaagttgtcattgtgctattcaattactcttcaacaacaacaacaacaacccagtataatcccactagtggggtttggggagggtagtgtgtacgcagaccttacccctaccttggggtagagaggctgtttccgatagaccctcggctccctccctccaagaactccccaccttgctcttggggtgactcgaactcacaacctcttggttggaagtggagggtgcttaccactagagcaacccactcttgtcttcatatagagcttgttggattgtatctcgtgaattttataaacttccttatcgaaagatgggatgttgttatctatttattatgcctttatactctcctttaagtgcaggctagatacatttccctgctaagcacgtcaaaattctttgatgatgatggctgtgatactttaacaaactaatatagtctgttccgttcgtgttgaattgtgtagaactcctcatttacaaaatgaagctgttagagatgaaaccgtttgttttttaattgtctttaacacagtttccctataggcatagtcgattaatcatttttcttgttatcttgtaggaggatgttctttttgcccgaattcgaacaactggtgtcgttgaaatacagttcaggtgaatttcacgaatccttgaacggtccttgtatcgatttaccaaaacctcacttttagtggtttatatacattttgatctcttaatgcagtgttggtttatatgcttttctatgtgtcaagtcttagccaccctgtttaaatgttattgaactagttaatgtgcagttgcttgggtcatcttcattaggttctgcatatttatgcatatctcttccccaaccttcatctagtctctatgtccctcatctattgactaagtgcctaatcattaggtgcatttatatgtccttaaatttcttgattattaatcattttatacttgtaacacaccggttcttgtggtcctccaatgatgcaattgcttatgaccttgtctatttgtccccttccccccttttaagacactttcgtattccatctagttttgattaaaggcttaataactactaaaatgtttgcagcccagttggagagaacaaaaaaagtggagaagtatataggctttttgatgttggaggtcagagaaatgagagaagaaagtggattcatctatttgaaggcgtcacggcagtaatattttgtgccgctattagtgagtaagatttctgctcctagctttctgatatttctaagatcatttgcttgaagtaaatccaatttctgacttgatcataagtagcaatacgtggatcatgtggctttccagattgaggaagaaaattaaagttagttaatgtaggtcaaaaggattttgaatttgtgggtaaaggaaatgaggcaattgcatacttgttattgttctattttttttccgttacttattattgttctattattcacttcaggtatgatcaaactctatttgaggatgaaagaaagaaccgaatgatggagagcaaggaactctttgagtgggtcttaaagcagccatgttttgaggtcagttttcgttgtctactagaagaccgatggttggttgcgaactcgaattttataatccatctttatcaacatgtacttacggctcttctagttatatgcttttgcatcacctttttcatcaaaaaagaaaaaataaacttttgcatcaccttttttgggtttttaaaaaaacaaatgaaaccagttatgaagcttacgctttattgtttgtgcagaaaacttccttcatgctatttctcaacaaatttgatatatttgagcagaaggctctgaaagtaagtacatattatctagtggtggtgtctgtcgttggaaccatcctttttcgtctaagaatgatatcccatggttatataggtgcctctgaacgtctgtgagtggtttaaagattaccaatcagtttcgacaggcaaacaagagattgagcatgcttatgagtaagcttctcatgtgcagcacttttatatatgagtacatgttactgatttcgctgatgcattcgttgggctctataaaacatggttgctttaagacatttgtaactcgatacacgcacacacagagacaccacatttattcgctcaacgatcttaaatgcttagacactatatcatggttgttaattcatatattctttagctattttcggtctattttctgctttttggctctacaccattattttcggtcttggggatgtgggggtagagccgatactctttcctttaaaactgttgctcaattcatgtgctgaacgaacgttctatatttcaatgatctactctttaggtttgtaaagaaaaaatttgaggagtcatatttccaatgcactgcaccagatcgtgtggaccgggtctttaagatatacagaaccacagcccttgatcagaagcttgttaagaggacattcaaactggtagatgagacgctgagaaggagaaacc

SEQ ID NO: 38 - protein, 366 aa, Nicotiana tomentosiformis
NQADDEENTQTADIERRIEQETKADKHIQKLLLLGAGDSGKSTIFKQIKLLFQTGFDEAELKNYIPVIHANVYQTIKVLHDGSKELAQSELEASKYLLSAENKDIGEKLSEIGGRLDYPHLTKDLVQDIEALWKDPAIQETILRGNELQVPDCAHYFMENLQRFSDINYVPSKEDVLFARIRTTGVVEIQFSPVGENKKSGEVYRLFDVGGQRNERRKWIHLFEGVTAVIFCAAISEYDQTLFEDERKNRMMESKELFEWVLKQPCFEKTSFMLFLNKFDIFEQKALKVPLNVCEWFKDYQSVSTGKQEIEHAYEFVKKKFEESYFQCTAPDRVDRVFKIYRTTALDQKLVKRTFKLVDETLRRRN

SEQ ID NO: 39 - DNA, 6005 bp, Nicotiana tabacum
aatcaagccgatgatgaggaaaatactcaggtatgagttatgcgaaatagatttggaattttggtaattgtccatagtgctgtctttaagctaggagagcaaagaatctattttcatcttgcttaatgcataagcaccagtgtccttactagatcatgcgtctacagactgcagatatagaaagacgtattgagcaagaaacaaaggcggacaagcatattcagaaacttcttctacttggtaaatcagaaaaattagtcttcaattgtcctatgatctagtcatttcttactgttagcttttccttttatgtcaccatatcaatgttgtgattcacgatttctatagacatctttcacctaatcctgttaaatttgaggaaagcaatagattttgaacttttttgaagcttactgccgtgtgttttgccgtatgattaaacttactgttgtgtgttttctggtctatgtcctgtttgactgtgtttgctgaatgctgatctttctgttgtttcaaggtgccggagattcggggaagtccactatttttaagcaggtaagagatcttatgattaatctttcacgatttcattattctctaaatgttcatggtaatgtatttcttttaactccagtgtcctctatttattttgccatgcagataaaacttttgttccaaactggctttgatgaggcagagctaaagaactatatccctgtcattcatgccaatgtctatcagacaataaaagtacggaatacttgaaagggtgtgttggttatttccctttttgcaaaatagctgctgcttgttagagacgtgcatatatagaaatatcattcacatgctttataatgcgggttgattagtaatgtcatggaaactgacattcttacatggtgggtgtttggtgacatgacaggtattacatgatggatcgaaggaattagctcaaagtgaattagaggcctcaaagtatcttctatcagctgaaaataaggtttgtcttattcgttcattgtgcaatttccttgcttttcttgcttattattaccagatatttgggctatcatcttgaagtcatttagccaagggaaacccttgaaagtgaggacatgtgattgcttgtcttgcttagctgcatgaacataataacgtatttctttgcaccaggatatcggcgagaagctttcagaaattggaggcaggttggattatcctcacctgactaaggatctggtgcaggatattgaagctctttggaaagatcctgctattcaagtaagccctttgatgactttatttcagtgtcaagtacttttttaaggcttgatgatttggattatgataattgtttttacttatataattaaatgattattgataattgaaacaatcaattcattgaagtcaatcaatcaactatgcctcaatccgtagctagtatttaaaaaaaaacccaaactagtggggttttcgcgatgatcttctatatttctgctctattggggccatttcacatttcatttcaataccactttttctttgtctgtaagtcaagttcagtagttctacaaaactagaggtttcttaaatctcactcttattcctaatcctaatgttgaaaaccagcaactactgatccttattcctatatagttggagttgtctatttggatcatttgtttccatcatgttctaatcttaactcagttcacaatattcctagatatcatagatcttttagataagcaccctagggtgtggcctagtggtcaataaagtgggtgcgaaactgggatcaaattccgccagagataaaaagcattaggtgtctcttcctatttgcgtaagccttggtggatagagttactcaatacctatgctggtgggaggtattaggaactcagtggtatagtcgaggtgagcgcaagttggtccagacaccaccgttataaaggaaaatctttttagataacttctctacgtgtgattctagaattaccttgtgcctttttagcaacttcaaccactaaagtcatacctacaaattggtgaatatggagggatgacttaataatctcaccttctcacattttattctgtatgtatgttacttgcactttctctgcctaagcatgagtgggcagagttacctagtacctattttgatgggcggtaacaagtatcggtaaaagagttgaggtgcaagtaaaatggcccaaaaaccaccttcataaaaaatgtatgttacttgcacctactgtctgagttgatcattttaactttgtctggatttgtaagattgcaaattcgttataactttcgcatttctaagacactcatcttgaggatacgttgggccttaaccttcattcccacattaccggtctttgcctttcactttattggatatgttcctatcacatagttttcttgtagtatacctccacatcaatcatctgatctaactctatgtgctacatctttatctgtaatgccattctcccagaagattgaacccatatatttgaatagtttgcattaaagcaccacattcattcactttaccttcattcttcttctgcgggcttcactggcaatgcatataatactgtcttactttacttatcccaaaatccttattctctagatcgcttctccacagttctagcctttaattttatttaagtaaatcacttggaatttgattttttcatacaatggccaatcagagattctgattaatcagcctgatggtgaaaatccaaactacgtgaacccttaatcttttatcgagtatcccagaaaagattaacttttcattagtctgatagagttttctttctttcttgtaggaaactattttacgtggtaatgagctccaggttccagattgtgcccattatttcatggaaaacttgcagagattttcagatataaattatgtcccatcaaaggtaagaaatagagactggatcatgtatgcttttcgtttctgtataccgattattttaattgaaatatatggaactagaatgctattgaaataaatagaatgtaggaagtcttgagactctgagttagtatgttttacagttctaccagtagaacagaagattaacaacaagttgttcattggtcgtataccaactctttaatcaatgaggtattttccaattaagagttcgctttctttgcaatagtaaaaatctgttctacttttcatattgaatttacgaagatatcaaagccatctgtactttacccaatgataaaactatcctctacatgatgttgaattcacagttgttgatagcagttttggctcagatgcattattagatttattttaccattaggattttggtacaaggggtgtctatgttaaccgaaaagaatactaatcttgacatgcatagctttggcaggatgggccaaaccagttttctttctgatctcattttaatccacccgtttgacacccctagtgttggtcaaatatttcaaaattcaaactgcaactttctagagtcaaatacgacgtcaagcgcccaagatacttgctattacacagtacacttgcccttgcttgtcacttatgcctgtatctgattttctattacactagtggtgttctttctataggcagtgtaagtgtaggaagtacaagtgttgcctgtctattgttggtagagaaaagtgtggtccatcaattaagttctcattgtgctattcagttactcttaattgctataatttctttcttcatatagagcttgttggattgtatctcgtgaattgtataaacttccttatcaaaagatgggatgttgttatctatttattatccctttatacactcctttaagtgcaggccagatacatttccctgccaagcacgtcaaaatcctttgatgatgatggctgtgatactttaacaaaggaatactgtttgttccattcgtgttgaattgtgtagaactcctcatttacaaaattaagctgttagaatggaaaccgtttgttttttcagtcgtctttaacacagtttccctatatgcatagtcgagtaatcatttttcttcttatattgtaggaggatgttctttttgcccgaattcgaacaactggtgtcgttgaaatacagttcaggtgaacttcacgcatccttgaactgcccttgtatcgattaactaaaaccttacttttagtggtttatatacattttgatctcttactggagtgttggtttatatgcttttctatgtgtcaagtcttagccaccctgtttaaatgctattgatgtagtttaatgcagttgcgggttatcttcattaggttctgcctacttatgcatatctcttccccaaccttcgtctagtctctttgtccctcatctattgactaagtgcctaatcattaggagcatttatatgtccttaaatttgttgattattaaacattttatacttgtaatgcactggttcttgtgtcctgcacgtaagacacttttgtattctatctagtttgggtgaaaggcctaataactactaaaatatttgcagcccagttggagagaacaaaaaaagtggagaagtatataggctttttgatgttggaggtcagagaaatgagagaagaaagtggattcatctatttgaaggtgtcacggcagtcatattttgtgccgctattagtgagtaagatttctgctcctagctttctgatatttctacgatcatttgctttaagtaaatccaatttctacttcatcataagtagcaatacagggatcgtgttgctttccagactgaagaagaaaattaaagttagttaatgtaggtcaaaaggattttgaatttgtcagtaaaggaaatgaggcaactgcatacttcttgttgttctattttttttttcagtcacttattattgttttctattattcacttcaggtatgatcaaactctatttgaggatgaaagaaagaaccgaatgatggagaccaaggaactctttgagtgggtcttaaagcaaccatgttttgaggtcagtttttcgttgtctactagaagaccgatgcttggttgcgaactcgaattttatatccatctgtatcaacgtgtacttatggctcttctagttctatgcttttgcatcacctttttcatccaaaaagaaaaagaaacttttgcatcacctttttggggtttttcgaaaaaacaaatgaaaccaattatgaagcttacactttattgtttgtgcagaaaacttccttcatgctatttctcaacaaatttgatatatttgagcagaaggctctgaaagtaagtacatattatctagtgatggtgtctgtctttggaaccattcttttcgtctaagaatgatatcccatggttataggtgcctctgaacgtctgtgagtggtttaaagattaccaaccagtttcaacaggaaaacaagagattgagcatgcttatgagtaagctttctcatgtgcagcacttttatacatgagtacatgttactgattccactgatgcattcgttgggctctataaaagtacttttttggtcccaagacatatgtaagctcaatacacgcacacgcatagacacacacattcatttgctcaaagatttttcctgcttagacactatatcatggttgttaattcatctattctttagctattttcagtctattttctgctttttggctctaccattattttgggtcttggggatgtcgattgtcggggtagagcccttactctttcctttaaaactgttgctcaattcatgtgctgaatgaacgttctatatttcaatgatctactctttaggtttgtaaagaaaaaatttgaggagtcatatttccaatgcactgcaccagatcgtgtggaccgggtctttaagatatacagaaccacagcccttgatcagaagcttgttaagaagacattcaaactggtagatgagacgctgagaaggagaaacc

SEQ ID NO: 40 - protein, 366 aa, Nicotiana tabacum
NQADDEENTQTADIERRIEQETKADKHIQKLLLLGAGDSGKSTIFKQIKLLFQTGFDEAELKNYIPVIHANVYQTIKVLHDGSKELAQSELEASKYLLSAENKDIGEKLSEIGGRLDYPHLTKDLVQDIEALWKDPAIQETILRGNELQVPDCAHYFMENLQRFSDINYVPSKEDVLFARIRTTGVVEIQFSPVGENKKSGEVYRLFDVGGQRNERRKWIHLFEGVTAVIFCAAISEYDQTLFEDERKNRMMETKELFEWVLKQPCFEKTSFMLFLNKFDIFEQKALKVPLNVCEWFKDYQPVSTGKQEIEHAYEFVKKKFEESYFQCTAPDRVDRVFKIYRTTALDQKLVKKTFKLVDETLRRRN

SEQ ID NO: 41 - DNA, 9781 bp, Nicotiana tabacum
ggtaccaagaacagtagcgaaaatattctttgagcaataaatatttatattaaattttaaaaatttaatttttgaaacaatattagttcatgctccctataaatattaaattaattaatttaattcagtttgttaaattaaagtaagaagatatattattaaaatatttctatctatgcgcatggcttgtgtttaattgatttagtatcagcatttatgtcatatttttggtaataaatagacaaaaaagaaagggaaaaaataaaaagtgaatgactctatttttttatttgttagtcaaagttgcaaccttgacctggttaggtacactctattttctgtctctgtctctgtgtctgtctttgtctctccaaagcaattgtttctgagcaccggcaaacttctccctttatctctctctctcagatcactgcttcatctaacagatcctcttaggtcagtgcattttttagctcaatctttaacttaatatacacttctgtatatataagtgtaaagttgctgtccccttttttaatttaatttgtttgtgggtttaatttccaagggttttagggctaggattgcttaagaattcaagaatgttggtaaatagggtgttcaagttgactgtgttaaatgcaaatgaagtggaaatgtaaagtgggtagttgtattttatgtttatatatacatacaccttaatgtataaaatgtaaagtttctgctttatttaaatgcttatggggttaatttccaaaggttttagggtttggagcgtttcagaatcaagaatgttagtgaacagggtgttgaatttactattttaactgtaaaagaagaggaaattagaaagtgggtagttttatttatgttcttatctaacttatatttattttcaggggatggggtaaatagtgttaatcttggaaaattaaaaaatggggatgtagtcacttctttcttaatttgcatgtggttttgatgtgtggtttaatttcctgctcaatagtcataatatcatgaaatgtatttaccaacattaaaatgaacgggttcgaactcggtggaatgtgtataaaggatagttgggatacgcacactgcacaagctggcccagaaaccactcttatagaaagatagaggattcatgcagctgatccaatctaactaaagattgaggcttagttaattgattgattaatgatgcgttgtcgtttgcgaataaatggaactaggttggttttggggccttagggagtgatactaactggcaactattaattattactgtaggaagaaacactacctctcaaaaaaattgctttctctatggattagaaacataacaattaaaatttagttgttacactgtcagtcttgatggtttgccaataaacatttcatccacaagtttaccatcaattctatagcaggttccaacctttctgagtgttcttccaacaatgaattttgaactttagtttctaaatcgctcacataacatattcatatattccttctgtataattgccttatttaactctgagaggttttttttgtgtgtgtgtgtgggtggggggttatcttggacttttatttgactgagatgcaaactgaaaccacggagacttattttggttattccttaaattcaactgaactggacgtgcagtaaagtctttttccgagttaattaccttattatcttatttcttgtctgcttctgtgaaaataaggtatacatggacatcataaacgagttgctacaaaagatttgagatctctagcttgactatcacacaggcctatgctgtgtgtggtattagaaaacatgggcttgttgtgcagcagaaacaaaggctacaatcaagccgatgatgaggaaaatactcaggtatgagttttacgaaatatatttggaatttttggtaattgtccgtagtgctgtctttaagctaggagagcaaagaatctattttcatcttgcttaatgcataaccaccagtgtccttactagatcatgtgtctacagactgcagatatagaaagacgtatcgagcaagaaacaaaggcggacaagcatattcagaaacttcttctacttggtaaatcagaaaaattagtcttcaattgtcatatgatctagttatttctcactgtttgcttttcctttttatgttcaccatatcagtgtagtgattcacgatttctatagacatctttcaccttggacttgttaaatttgaacttttcttttcctcgaagcttactgttgtgtgttttgcgtatgattaaacttactgctgtgtgttttttggtctatgttctgtttgactgtgtttgctgaatgctgatctttctgttgcttcaaggtgccggagattcggggaagtccactatttttaagcaggtaagagatcttatgattaatattttacgattcattattctctaactgttcatgttaatgtatttcttttaactccagtgtcctctatttatttttgccatgcagataaaacttttgtttcaaactggctttgatgaagcagagctaaagaactatatccctgtcattcatgccaatgtctatcagacaataaaagtacggaatacttgaaagggtgtgttggttatttctctttttgcaaaatagccgctgcttgttagagacgtgcatatatagaaatatcattcacatgctttataacgaggtttgattactaatgtcacggaaactgacattcttacatggtgggtgttcggtgacataacaggtattacatgatgggtcgaaggaattagctcaaagtgaattagaggcctcaaagtatcttctatcagctgaaaataaggtatgtgggctatcatcttgaagtcatttagacaagggaaacccttgaaattgaggacatgtgattgcttgtcttgcttagctgcatgaacataataccgtatttctttgcaccaggatatcggcgagaagctctcagaaattggaggtaggttggattatcctcacctgactaaggatctggtgcaggatattgaagctcttcggaaagatcctgctattcaagtaatcttgcttcgcttaagccctttgatgactttatttcagtgtcaagtacttttttaaggcttgatgatttggatttgtttttacttatataattaaatgattattgataattgaaacaatcaattcattgaagtcactcaatcaactatgcctcaatccgtaactagtattctagtggggttttcgcgatgatcttctatatattctgctctattggggccatttcacatttcattccaataccactttttctttgtctgtaagtcaagttcagtagttctacaaaactagaggttttttaaatctcactcttattcctaatcctaatgttgaaaaccagcaacaactgatccgtattcctatataattggagttgtctatttggataatttgtttccatcatgttctagtcttaactcagttcacaatattcctagatatcatagctctttttagacaagcaccctagggtgtgacctagtggtcaataaagtgggtgcaaagctgggatcaaattccgccagagacaaaaaacagtaggtgatctcttcccatttgcataagccatggtggatagagttactcaatacctatgctggtgggaggtatcgggaactcggtggtataatcgaggtgagcgcaagttggtccagacaccaccgttataaaagaaaatctttttagataacttctctccgtgtgattctagaattacctggtcccttttttagcaccttcaatcactaaagtcatacctacatattggtgaatttggagggatgacttagtaatctcaccgtctcacattttattctgtatgtatgttacttgcaccttctctgcctaagcatgggtgggcagaatgacctagtacctattttgatgggcggtaacaagtatcggtgaaactgttgaggtgcaagtaaagtggcccaaacgccaccttcataaaaaatgtatgttacttgcactttctgtctgaggtgatcattttaattttgtctacatttgtaagattacaaattcgttataactttcgcatttctaagacactcatcttgaggatacgttgggccttaaccttcattccctcattaccggtctttgcctttcactttattagatatgttcctatcacatagttttcttgtagtacgcctccaccacaatcatctgatctaactctatgtgctgcatctttatctgtagtgccattctcctagaagattgagcctatatatttgaataatttgtattaaagcaccacaatcgttcactttaccttcattcttcttgtgtgggctacactggcaatgcatataatactgtcttacttttatttatcccaaaatccttgttctctagatcgcttctccatagttgtagcctttagttttattgaagtacatcactttagaatttgattttttcataaaatggccaatcagagattctgattaatcaagctgatggtgaaaaatccgaactacgcaaatccataattttctatgagtatcccagaaaagattaacttttcattactctgatagagttttctttctttcttgtaggaaactatattacgcggtaatgagctccaggttccagattgtgcccattatttcatggaaaacttgcagagattttcggatataaattatgtcccatcaaaggtaagaaatagagactggatcatgtatgttttttgtttctgtattccgattatattaactgaaatatatggaactagaatgctattgaaataaatagaatataggaagtcttgagacactaagttagtatgttttacagttctaccagtagaacagaagattaacaacacattgttcattggttgtataccaactctttaatcaatgaggtattttccaattaagagttcgctttcattgcaataataaaaatctgttctacttttcatattgaatttacgaagaaatcaaagccatctgtactttaacccaatgataaaactatcctctacatgatgttgaattcacagttgttgatagcagttttggctcagatgcattattaaaactattttacccttaggattttggtcctaggggtgtctatggcaaagctatgcatgccaatgtgtaaatgtgtacacacattataagaatcagacctgagaaatgagagacaagtaggagatggtaagctaaacaagtaaatgtgtaaatgacaaatattttttcattttacatgctcttaatccggtggttcttatacatgtattgttattagttatagaataaatttaatacccaattgttaatacaaataattactagattgtttttaaattgttctggaatagaggcggaactatgaataatggagcattatcatataacttaagtgatacaagatagggttgcagcacaattaccaatccctatttaggccataaccaagattatttccaaattatcaaaacgacccttacgtgttcccaaacctacggcttgtttcaagcatccttatccttaaatctattatactgttcatagaactctagtttctaaccatatattatatcaacattggctttttgtggtgatgcttctcgaaagtgctataattgatcgcgcttttatttattaattaatccttattttaaatgatatggaatatttttagattgctgggtctacatacctttaaataacctgctttgttttgctttgctatattttttctttttgaagagataatgttcctcttatcattttgtcaattttaagggttggtgggttcctttctgtccccttcatccattgctcatcgttttccaagctatctctttcgttcattttcttttttcttggtgtaactgagttggtgagtttttgcctttctattagaagtgatgatgagtatttaaactgtaaagaattaagtaatgatagtttacaatgaatacgatgccgcacatttaccttaaatttataagtgggaatctggaattttcaactttggatataccagaaaaagggccacatataatgggttatgtttttatagtttaagtggtatactgtttaaccaaaaaataagatctttgttcgaagcctatttttagaagaactcggattactgataaccaaataatttcttacttcctcagtaactcacttaaaaggaaataaagttgacaaaaagtaactaaaaagaaagaataattgattgcactgaataagcaagagaaagtgaatatatttcgatagtactcaaaacgtgtctttacagatgagattctcccccttatataggatcttgacatgcatagctttgccaggacgggccaaacccgttttctttctgatctcattttaacccacccgtttgacactcctagtgttggtcaaatatttcaaaattcaaactgcaattttctagagtcaaatacgatgtcgagcacccaagatacttgctattacacagtacagttgcccttgcttatcgcttatgcctgtatctaattttctattacactagtggtgttctttctataggcagtgtaagtgtaggaagtgcaagtgttgcctgtctattgctggtaaagaaaagtgtggtccatcaatcaagttgtcattgtgctattcaattactcttcaacaacaacaacaacaacccagtataatcccactagtggggtttggggagggtagtgtgtacgcagaccttacccctaccttggggtagagaggctgtttccgatagaccctcggctccctccctccaagaactccccaccttgctcttggggtgactcgaactcacaacctcttggttggaagtggagggtgcttaccactagagcaacccactcttgtcttcatatagagcttgttggattgtatctcgtgaattttataaacttccttatcgaaagatgggatgttgttatctatttattatgcctttatactctcctttaagtgcaggctagatacatttccctgctaagcacgtcaaaattctttgatgatgatggctgtgatactttaacaaactaatatagtctgttccgttcgtgttgaattgtgtagaactcctcatttacaaaatgaagctgttagagatgaaaccgtttgttttttaattgtctttaacacagtttccctataggcatagtcgattaatcatttttcttgttatcttgtaggaggatgttctttttgcccgaattcgaacaactggtgtcgttgaaatacagttcaggtgaatttcacgaatccttgaacggtccttgtatcgatttaccaaaacctcacttttagtggtttatatacattttgatctcttaatgcagtgttggtttatatgcttttctatgtgtcaagtcttagccaccctgtttaaatgttattgaactagttaatgtgcagttgcttgggtcatcttcattaggttctgcatatttatgcatctctcttccccaaccttcatctagtctctatgtccctcatctattgactaagtgcctaaccattaggtgcatttatatgtccttaaatttcttgattattaatcattttatacttgtaacacaccggttcttgtggtcctccaacgatgcaattgcttatgaccttgtctatttgtccctttccccccttttaagacacttttgtattccatctagtttggattaaaggcttaataactactaaaatgtttgcagcccagttggagagaacaaaaaaagtggagaagtatataggctttttgatgttggaggtcagagaaatgagagaagaaagtggattcatctatttgaaggcgtcacggcagtaatattttgtgccgctattagtgagtaagatttctgctcctagctttctgatatttctaagatcatttgcttgaagtaaatccaatttctgacttgatcataagtagcaatacgtggatcatgtggctttccagattgaggaagaaaattaaagttagttaatgtaggtcaaaaggattttgaatttgtgggtaaaggaaatgaggcaattgcatacttgttattgttctattttttttccgttacttattattgttctattattcacttcaggtatgatcaaactctatttgaggatgaaagaaagaaccgaatgatggagaccaaggaactctttgagtgggtcttaaagcagccatgttttgaggtcagttttcgttgtctactagaagaccgatggttggttgcgaactcgaattttataatccatctttatcaacatgtacttacggctcttctagttatatgcttttgcatcacctttttcatcaaaaaagaaaaaataaacttttgcatcaccttttttgggtttttaaaaaaacaaatgaaaccagttatgaagcttacgctttattgtttgtgcagaaaacttccttcatgctatttctcaacaaatttgatatatttgagcagaaggctctgaaagtaagtacatattatctagtggtggtgtctgtcgttggaaccattctttttcgtctaagaatgatatcccgtggttatataggtgcctctgaacgtctgtgagtggtttaaagattaccaatcagtttcaacaggcaaacaagagattgagcatgcttatgagtaagcttctcatgtgcagcacttttatatgagtacatgttactgatttcgctgatgcattcgttgggctctataaaacatggttgctttaagacatttgtaactcgatacacgcacacacagagacaccacatttattcactcaacgatctttcctgcttagacactatatcatggttgttaattcatatattctttagctattttcggtctattttctgctttttggctctacaccattattttcggtcttggggatgtggggttagagccgatactctttcctttaaaactgttgctcaattcatgtgctgaatgaacgttctatatttcaatgatctactctttaggtttgtaaagaaaaaattagaggagtcatatttccaatgcactgcaccagatcgtgtggaccgggtctttaagatatacagaaccacagcccttgatcagaagcttgttaagaagacattcaaactggtagatgagacgctgagaaggagaaacctctttgaagcaggtttattatgaaattctttaaattttggaaacagaaatgttcataccctgaaagatgcatacaagtgcgaggttcaaacacagaaaaataggctactggcgtatcatatccaattccactatttaaagttttgccaatgttaggtctctaagcacatatttctttctatattcctggttgtatttaccgagcagatgttccaaaacaaaaaaatgatattcaagtatattcgattgatgttcattttgttgaatctcacatctcaagttgtacatgcatcgttagtcaaccttttgcaatctcgtgatttatttagtttattgtatagctcttgcccgcttatgttatcagagtgttgttattccagaacttgttcaagattttgactattttccatgatatttctgcatctgttttgttgcacaatcctagattccactttggaacgtatactacctacataaggtaattgcttgctgaatttttcctttcaacttttatggtctttgaatggtgtggttttagctcccatttggtttttgtg

SEQ ID NO: 42 - protein, 384 aa, Nicotiana tabacum
MGLLCSRNKGYNQADDEENTQTADIERRIEQETKADKHIQKLLLLGAGDSGKSTIFKQIKLLFQTGFDEAELKNYIPVIHANVYQTIKVLHDGSKELAQSELEASKYLLSAENKDIGEKLSEIGGRLDYPHLTKDLVQDIEALRKDPAIQETILRGNELQVPDCAHYFMENLQRFSDINYVPSKEDVLFARIRTTGVVEIQFSPVGENKKSGEVYRLFDVGGQRNERRKWIHLFEGVTAVIFCAAISEYDQTLFEDERKNRMMETKELFEWVLKQPCFEKTSFMLFLNKFDIFEQKALKVPLNVCEWFKDYQSVSTGKQEIEHAYEFVKKKLEESYFQCTAPDRVDRVFKIYRTTALDQKLVKKTFKLVDETLRRRNLFEAGLL

SEQ ID NO: 43 - DNA, 1677 bp, Nicotiana tabacum
cactgcttcatctaacagatcctcttaggtatacatggacatcataaacgagttgctacaaaagatttgagatctctagcttgactatcacacaggcctatgctgtgtgtggtattagaaaacatgggcttgttgtgcagcagaaacaaaggctacaatcaagccgatgatgaggaaaatactcagactgcagatatagaaagacgtatcgagcaagaaacaaaggcggacaagcatattcagaaacttcttctacttggtgccggagattcggggaagtccactatttttaagcagataaaacttttgtttcaaactggctttgatgaagcagagctaaagaactatatccctgtcattcatgccaatgtctatcagacaataaaagtattacatgatgggtcgaaggaattagctcaaagtgaattagaggcctcaaagtatcttctatcagctgaaaataaggatatcggcgagaagctctcagaaattggaggtaggttggattatcctcacctgactaaggatctggtgcaggatattgaagctctttggaaagatcctgctattcaagaaactatattacgcggtaatgagctccaggttccagattgtgcccattatttcatggaaaacttgcagagattttcggatataaattatgtcccatcaaaggaggatgttctttttgcccgaattcgaacaactggtgtcgttgaaatacagttcagcccagttggagagaacaaaaaaagtggagaagtatataggctttttgatgttggaggtcagagaaatgagagaagaaagtggattcatctatttgaaggcgtcacggcagtaatattttgtgccgctattagtgggtatgatcaaactctatttgaggatgaaagaaagaaccgaatgatggagaccaaggaactctttgagtgggtcttaaagcagccatgttttgagaaaacttccttcatgctatttctcaacaaatttgatatatttgagcagaaggctctgaaagtgcctctgaacgtctgtgagtggtttaaagattaccaatcagtttcaacaggcaaacaagagattgagcatgcttatgagtttgtaaagaaaaaatttgaggagtcatatttccaatgcactgcaccagatcgtgtggaccgggtctttaagatatacagaaccacagcccttgatcagaagcttgttaagaagacattcaaactggtagatgagacgctgagaaggagaaacctctttgaagcaggtttattatgaaattctttaaattttggaaacagaaatgttcataccctgaaagatgcatacaagtgcgaggttcaaacacagaaaaataggctactggcgtatcatatccaattccactatttaaagttttgccaatgttaggtctctaagcacatatttctttctatattcctggttgtatttaccgagcagatgttccaaaacaaaaaaatgatattcaagtatattcgattgatgttcattttgttgaatctcacatctcaagttgtacatgcatcgttagtcaaccttttgcaatctcgtgatttatttagtttattgtatagctcttgcccgcttatgttatcagagtgttgttattccagaacttgttcaagattttgactattttcccatggaaaaaaaaaa

SEQ ID NO: 44 - protein, 384 aa, Nicotiana tabacum
MGLLCSRNKGYNQADDEENTQTADIERRIEQETKADKHIQKLLLLGAGDSGKSTIFKQIKLLFQTGFDEAELKNYIPVIHANVYQTIKVLHDGSKELAQSELEASKYLLSAENKDIGEKLSEIGGRLDYPHLTKDLVQDIEALWKDPAIQETILRGNELQVPDCAHYFMENLQRFSDINYVPSKEDVLFARIRTTGVVEIQFSPVGENKKSGEVYRLFDVGGQRNERRKWIHLFEGVTAVIFCAAISGYDQTLFEDERKNRMMETKELFEWVLKQPCFEKTSFMLFLNKFDIFEQKALKVPLNVCEWFKDYQSVSTGKQEIEHAYEFVKKKFEESYFQCTAPDRVDRVFKIYRTTALDQKLVKKTFKLVDETLRRRNLFEAGLL

SEQ ID NO: 45 - DNA, 1427 bp, Nicotiana plumbaginifolia
aaatatttgagatctctagcttgactatcacacaggcctatgcgctgtgtggtattagaaaacatgggcttgttgtgcagcagaaacaaaggctacaatcaagccgatgatgaggaaaatactcagactgcagatatagaaagacgtattgagcaagaaacaaaagcggacaagcatattcagaaacttcttctacttggtgccggagattcggggaagtccactatttttaagcagataaaacttttgttccaaactggctttgatgaagcagagctaaagaactatatccctgtcattcatgccaatgtctatcagacaataaaagtattacatgatgggtcgaaggaattagctcaaagtgaattagaggcctcaaagtatcttctatcagctgaaaataaggatatcggcgagaagctttcagaaattggaggcaggttggattatcctcacctgactaaggatctggtgcaggatattgaagctctttggagagatcctgctattcaagaaactattttacgtggtaatgagctccaggttccagattgtgcccattatttcatggaaaacttgcagagattttctgatgtaaattatgtcccatcaaaggaggatgttctttttgcccgaattcgaacaactggtgtcgttgaaatacagttcagcccagttggagagaacaaaaaaagtggagaagtatataggctttttgatgttggaggtcagagaaatgagagaagaaagtggattcatctatttgaggatgaaagaaagaaccgaatgatggagaccaaggaactctttgagtgggtcttaaagcaaccatgttttgagaaaacttccttcatgctatttctcaacaaatttgatatatttgagcagaaggctctgaaagtgcctctgaacgtctgtgagtggtttaaagattaccaaccagtttcaacaggaaaacaagagattgagcatgcttatgagtttgtaaagaaaaaatttgaggagtcatatttccaatgcactgcaccagatcgtgtggaccgggtctttaagatctacagaaccacagcccttgatcagaagcttgttaagaagactttcaaactggtagatgagacgctgagaaggagaaacctttttgaagcaggtttattatgaaattctttaaattttggaaacagaaatgttcataccctgaaagaagcatacaagtgcgaggttcaaacacagaaaaataggctactggcgtatcatatcatatccaattccactatttaaagttttgtcaatgttaggtctctaagcacatatttctttctatattcctggtggttgtatgttgtatttaccgagcacatgttccaaaacaaaaaattgatattcaagtatattcgatcgatgttcattttgttgaaaaaaaaaaaaaa

SEQ ID NO: 46 - protein, 372 aa, Nicotiana plumbaginifolia
MRCVVLENMGLLCSRNKGYNQADDEENTQTADIERRIEQETKADKHIQKLLLLGAGDSGKSTIFKQIKLLFQTGFDEAELKNYIPVIHANVYQTIKVLHDGSKELAQSELEASKYLLSAENKDIGEKLSEIGGRLDYPHLTKDLVQDIEALWRDPAIQETILRGNELQVPDCAHYFMENLQRFSDVNYVPSKEDVLFARIRTTGVVEIQFSPVGENKKSGEVYRLFDVGGQRNERRKWIHLFEDERKNRMMETKELFEWVLKQPCFEKTSFMLFLNKFDIFEQKALKVPLNVCEWFKDYQPVSTGKQEIEHAYEFVKKKFEESYFQCTAPDRVDRVFKIYRTTALDQKLVKKTFKLVDETLRRRNLFEAGLL

SEQ ID NO: 47 - DNA, 1362 bp, Pisum sativum
gcggccggtcgaccttgctttcaactttcacttcacactataatcccaaaaatctaacggcatattccatctctgcaaaacataaagacttccttttgcttcttttcggaaagtatgggcttagtctgtagcagaaatcggcgttatcgggattctgatcctgaagaaaatgcacaggcagcagaaattgaaagaagaatagagtcagaaacaaaggctgagaaacatattcagaaacttctactactaggtgcgggagagtccgggaaatctacaatctttaagcagattaaacttttgtttcaaactggctttgatgaggctgagctaagaagctacacaccagtcatttttgctaatgtgtatcagactataaaagtactgcatgatggggcaaaggagttggctcaaaacgatcttaattctgcaaagtatgttatatccgatgagagcaaggacattggtgaaaaactttcagaaattggaagcaggctggattatcctcatctcactaaggatcttgcaaaggaaatagagactctatgggaggatgctgccattcaggaaacatatgcccgtggtaatgaactccaagttcctgattgtaccaaatatttcatggaaaatttgcagaggttgtctgatgctaattacgttcctacaaagggggatgttttgtatgcaagagttcgtacaactggtgttgtggagatccagttcagccctgttggagaaaataagagaagtggtgaagtctatagactctttgatgttggtggccagagaaatgagaggagaaagtggatccatctttttgaaggagttacagctgttatattctgtgctgcaattagcgagtatgatcaaacactttttgaggatgaaagcaagaacagactgatggaaactaaggagctttttgaatggatcctgaagcaaccatgttttgagaaaacgtccttcatgttatttttaaacaagtttgacatatttgagaagaagatcctgaatgttccgctcaacgtatgtgaatggttcaaagattatcagccagtttcatcagggaaacaagagattgagcacgcatatgagtttgtgaagaaaaagtttgaggaattatacttccagagctctgctcctgaccgtgtagatcgcgtcttcaagatctatcgtaccactgcccttgatcagaaggttgtgaagaagactttcaagcttgttgatgagacgttgaggcggaggaatctttttgaagcgggattattatgaccatgcaacattgtgcataagataaaagggataaaattatttttacattgaagagctaatcagattttgggtatactaggtcgacgcggccgc

SEQ ID NO: 48 - protein, 384 aa, Pisum sativum
MGLVCSRNRRYRDSDPEENAQAAEIERRIESETKAEKHIQKLLLLGAGESGKSTIFKQIKLLFQTGFDEAELRSYTPVIFANVYQTIKVLHDGAKELAQNDLNSAKYVISDESKDIGEKLSEIGSRLDYPHLTKDLAKEIETLWEDAAIQETYARGNELQVPDCTKYFMENLQRLSDANYVPTKGDVLYARVRTTGVVEIQFSPVGENKRSGEVYRLFDVGGQRNERRKWIHLFEGVTAVIFCAAISEYDQTLFEDESKNRLMETKELFEWILKQPCFEKTSFMLFLNKFDIFEKKILNVPLNVCEWFKDYQPVSSGKQEIEHAYEFVKKKFEELYFQSSAPDRVDRVFKIYRTTALDQKVVKKTFKLVDETLRRRNLFEAGLL

SEQ ID NO: 49 - DNA, 1775 bp, Pisum sativum
cgcggccggtcgaccacctttcggcgctctttttcttttatcccatttttttcctccacgcacccccttttttctcattatttcttttcacaccctcatcaaccaccaccaccatatatgtttttctcttcccattattgccaacagtatatgcaaatcaaaaccatatcataaaaatttcttttttattttcattattattattataactgaacctgcatcactcaaatctaacaacacactttcaggtgaaatcaagttgattattgtgtatacatatattagagaagggcattgaattacagtgtgatttctgcgggagcttgagtagtcatcttctatgctgtgttttgtaacagaaaatatgggcttactctgtagcaaaagtaaccgttacaatgatgccaaagctgaagaaaatgcacagactgcagaaattgaaagaagaatagagttagaaacaaaggctgaaaagcatatcagaaaacttctactactaggagctggagagtcggggaagtccacaatatttaagcagataaaacttttatttcaaactggctttgatgaggcagagctaaaaagctatctaccagtcgttcatgctaatgtatatcagacaataaaattacttcatgatggatcgaaggagtttgcacagaatgatgttgatttttcgaagtatgttatatctactgaaaataaggacattggtgaaaagttatcagaaattggtggcagactggattatccacgtctcaccaaagaacttgcacaggaaattgagagtatctggaaggatgctgcaattcaggaaacatatgcccgtggtaatgagctccaagttccggattgtacgcactatttcatggaaaatttgcagaggctgtctgatgcaaattatgttccaacaaaggaggatgtcttacttgccagagttcgtactaccggtgttgtagagatccagttcagccctgttggagaaaacaagaaaagtggtgaagtctatagactgtttgatgtcggcggccagagaaatgagaggaggaaatggatccatctgtttgaaggagtttccgctgtaatattctgtgttgcgattagcgaatacgatcaaacactttttgaagatgagaacaagaacagaatgatggagacaaaggaactttttgaatgggtcctgaagcaacaatgttttgagaaaacatccttcatgttgtttttgaacaagttcgacatatttgagaagaagatcctggatgtcccacttaatgtatgtgagtggttcaaagattaccagccagtttcaaccgggaagcaagagatcgagcatgcatacgagtttgtgaagaaaaaatttgaggaatcatatttccagagcactgctccggatagcgtagaccgcgtgttcaaaatctataggaccactgcacttgatcagaaggttgtgaagaagacattcaagctcgttgacgagactttgagacgaagaaatctctttgaggctggcttgttatgaccagtgaatgagtcatgttttataagagggataaagtgttttttatagtgaagaggtgagatcagattttgggtatactaaacattaaatcgatttgttgattttatttctagtaaaatcttgttggagtgagtggatggagaaaagcctttatatagtgatcttcacactcatcttcaaagggtaaatttgtttcaagatttgatatcatgatttgtgattatgtttttatagaccaaaaaaaaaaaaaaaaaaa

SEQ ID NO: 50 - protein, 384 aa, Pisum sativum
MGLLCSKSNRYNDAKAEENAQTAEIERRIELETKAEKHIRKLLLLGAGESGKSTIFKQIKLLFQTGFDEAELKSYLPVVHANVYQTIKLLHDGSKEFAQNDVDFSKYVISTENKDIGEKLSEIGGRLDYPRLTKELAQEIESIWKDAAIQETYARGNELQVPDCTHYFMENLQRLSDANYVPTKEDVLLARVRTTGVVEIQFSPVGENKKSGEVYRLFDVGGQRNERRKWIHLFEGVSAVIFCVAISEYDQTLFEDENKNRMMETKELFEWVLKQQCFEKTSFMLFLNKFDIFEKKILDVPLNVCEWFKDYQPVSTGKQEIEHAYEFVKKKFEESYFQSTAPDSVDRVFKIYRTTALDQKVVKKTFKLVDETLRRRNLFEAGLL

SEQ ID NO: 51 - protein, 384 aa, Lycopersicon esculentum
MGSLCSRNKHYSQADDEENTQTAEIERRIEQETKAEKHIQKLLLLGAGDSGKSTIFKQIKLLFQTGFDEEELKNYIPVIHANVYQTTKILHDGSKELAQNELEASKYLLSAENKEIGEKLSEIGGRLDYPHLTKDLVQDIEALWKDPAIQETLLRGNELQVPDCAHYFMENLERFSDVHYIPTKEDVLFARIRTTGVVEIQFSPVGENKKSGEVYRLFDVGGQRNERRKWIHLFEGVTAVIFCAAISEYDQTLFEDERKNRMMETKELFEWVLKQPCFEKTSFMLFLNKFDIFEQKVPKVPLNACEWFKDYQSVSTGKQEIEHAYEFVKKKFEESYFQCTAPDRVDRVFKIYRTTALDQKLVKKTFKLVDETLRRRNLFEAGLL

SEQ ID NO: 52 - DNA, 1660 bp, Spinacia oleracea
ggcaggtctgaactactccactcaagtgaagactgcccaattcccaaattctaaaatccagtcaagcaaggctgtactctgtcacccaactacccaacacccaacaccaccgtccaccaccgtccaccactacggctgcagaatcaccgccattagcataagcagctgaaccctaatttacagaataattacaattacaattgcaattccatacgcttagcatgggactactttgcagcaagcatcaacattccaccaaacctgatgctgaaaatgcccaggcaacagggatagaaagaaggattgagcgagagactattgctgaaaagcatattcagaaactcttattacttggtgctggagagtccggaaagtcaacaatatttaagcagattaaacttttatttcagatgggatttgatgatgcagagttgaacagctatacacccgttattcatgccaatgtctatcagactatcaaattattgattgatggttccaaggaactggctcaaaatgaaacagattcttcaaagtatagcttgtcccctgataacaaggaaattggggacaagctgtcagaaattgggggcaggttggactatccacaactcaccaaagaactttctgaggaaatagaaaaaatatggaatgatccggcaattcaggaaactcatgcccgcagcagcgaactccaacttccagactgtgccaattatttcatggaacacctagacagactttctgatgtaaattatatccctacaaaggaagatgttctctatgcccgagtccgcacaacaggtgttgttgagatccagttcagtccagttggagaaaataagaaaagtggtgaggtatatagactttttgatgttggaggccaaagaaatgagcgaagaaagtggatccatctttttgaaggtgttacagcagtaatcttttgtgctgctataagcgattatgatcaaatgctctatgaggatgagaacaagaatcggatggttgaaactaaggagctttttgagtgggtcttgaagcagcgctgctttgagagaacatccatcatgctgttcctgaacaagtttgatattttcgagaagaaggttcagaaagttccactaagtacatgcgaatggtttaaggattaccagccagtttcgtctggacaacaagagattgagcatacctacgagtttgttaagaagaaatttgaggagctctattaccaatgcactgcccctgatcgtgttgatcgagttttcaagatttacagaacaactgctcttgaccagaagcttgtaaagaagactttcaaactgctagatgagactctcagaaggagaaaccttgttgaggcaggtttgttatgatacagaatggcaatttcggtgtgagtttgttaatagtatttggttctggggggttctgatcatatgttgaagtgtcaaattgaattaattaaaagagggaccagaattttttgacaccaaatttgactactgtctttacactacattacttttagagattacagtgttgagtccacatgtttgaagtttgaactctctgttacatatattgtcttgcctccatcctgttggagcgccagaataccttgtagcttaatatttcaatcagaagattatttattggccgc

SEQ ID NO: 53 - protein, 383 aa, Spinacia oleracea
MGLLCSKHQHSTKPDAENAQATGIERRIERETIAEKHIQKLLLLGAGESGKSTIFKQIKLLFQMGFDDAELNSYTPVIHANVYQTIKLLIDGSKELAQNETDSSKYSLSPDNKEIGDKLSEIGGRLDYPQLTKELSEEIEKIWNDPAIQETHARSSELQLPDCANYFMEHLDRLSDVNYIPTKEDVLYARVRTTGVVEIQFSPVGENKKSGEVYRLFDVGGQRNERRKWIHLFEGVTAVIFCAAISDYDQMLYEDENKNRMVETKELFEWVLKQRCFERTSIMLFLNKFDIFEKKVQKVPLSTCEWFKDYQPVSSGQQEIEHTYEFVKKKFEELYYQCTAPDRVDRVFKIYRTTALDQKLVKKTFKLLDETLRRRNLVEAGLL

SEQ ID NO: 54 - DNA, 1719 bp, Glycine max
tcgtgtttgttcttgttttcgctgctgacactatcctagtttttttgttaggtgaagtcaagcccgctaatgtgtgtacacattagagaagggcattgaaacaaagtgtgatttctggtggagcttgactagtcatcttctatgctgtcttttgtacagaaaatatgggcttactctgtagcagaaatcgccgttataatgatgctgatgctgaagaaaatgcacagactgcagagattgaaagaagaatagaggttagaaacgaaagggctgaaaagcatattcagaaacttctactacttggagctggagagtcagggaagtccacaatatttaagcagataaaacttttgtttcaaactggctttgacgaggcagaactaaaaagctacttaccagtcattcatgcaaatgtgtatcagacaataaaattactgcatgatggatcaaaggaatttgcccagaatgatgttgattcttcaaagtatgttatatccaatgaaaataaggaaatcggggaaaagttattggaaattggaggcaggctggattacccatatctcagcaaggagcttgcacaggaaattgagaatctgtggaaggatcctgcaattcaggagacatatgcccgaggtagtgagcttcaaattccagattgtactgattatttcatggaaaatttgcaaaggctgtctgatgcaaattatgttccaacaaaggaggatgttttgtatgcaagagtgcgtaccactggtgttgtagagatccagttcagtcctgttggggaaaataagaaaagtgatgaagtctatagactctttgatgttggcggccagagaaatgagaggagaaagtggatccatttgtttgaaggagtttcagctgtaatattctgtgctgcaattagcgagtatgatcagacactttttgaggatgaaaacagaaacagaatgatggagaccaaggaacttttcgagtggatcctgaagcaaccatgttttgagaaaacgtccttcatgttattcttaaacaagtttgacatatttgagaagaagatcctgaaagtcccacttaatgtatgtgagtggttcaaagattaccaaccggtttcaacagggaaacaagagattgagcatgcatatgagtttgtgaagaaaaaatttgaggaatcatatttccagagcactgctcctgatcgcgtagatcgcgtctttaagatctaccggaccactgcccttgatcagaaggttgtgaagaagactttcaagcttgttgatgagactttgaggcggagaaatctcttggaagctggcttgttatgagcactgaaccatacatgttataaaatgggataacaatatttttacattgaagaggtgaccagattttgggtatactaggcgattcaggtatactaaatattaaaatcgatttgttgatttttatttctaagttaatcttgtggagagaagaaaggccttgcttggagttgatatcataatctgtgatcatatttttatagattgaaagtcactaatcatatgatatatttcatactattagtgattatattttgcctctagtgttgttgtgttaatgtgcatacatgcatcatgcagattagatgcatgcacgcgtgtaaataatttggaaacgtgccatgtgtcatgtgctggctttgtcgagtctgaattcagaccttatattaaatttgctttt

SEQ ID NO: 55 - protein, 385 aa, Glycine max
MGLLCSRNRRYNDADAEENAQTAEIERRIEVRNERAEKHIQKLLLLGAGESGKSTIFKQIKLLFQTGFDEAELKSYLPVIHANVYQTIKLLHDGSKEFAQNDVDSSKYVISNENKEIGEKLLEIGGRLDYPYLSKELAQEIENLWKDPAIQETYARGSELQIPDCTDYFMENLQRLSDANYVPTKEDVLYARVRTTGVVEIQFSPVGENKKSDEVYRLFDVGGQRNERRKWIHLFEGVSAVIFCAAISEYDQTLFEDENRNRMMETKELFEWILKQPCFEKTSFMLFLNKFDIFEKKILKVPLNVCEWFKDYQPVSTGKQEIEHAYEFVKKKFEESYFQSTAPDRVDRVFKIYRTTALDQKVVKKTFKLVDETLRRRNLLEAGLL

SEQ ID NO: 56 - DNA, 1624 bp, Glycine max
ggcacgaggttgctttctagtttcgcttcacacttcacacttaacacttaacacttaacgtatccgccaaatctaggggcatattttaccaccacttctctgcaaaagagacccttttgcttcttttcagataatatgggcttagtctgcagcagaagtcgtcgttttcgtgaagctcatgctgaagaaaatgctcaggatgcagaaattgaaagaagaatcgagttagaaacaaaggctgaaaagcatattcagaaacttttactactaggtgctggagagtctgggaggtctacaatatttaagcagataaaacttttgtttcaaactggctttaatgaggctgagcttaaaagctacataccagtcgttcatgctaatgtgtatcaaacaataaaagtactgcaggatgggtcgaaagagttggcgcagaatgactttgattcttcaaagtatgtaatatctaatgaaaaccaggacattggtcaaaagctctcagaaattggaggcaccctggtttacccgcgtcttaccaaagagcttgcacaggaaatagagactatgtgggaggatgctgcaattcaggaaacatatgcccgtggtaatgaactccaagttccagattgtgcccattatttcatggaaaatttggagaggctgtctgatgcaaattatgttccaactaaggaggattttttgtatgcaagagttcgtacaactggtgttgttgagatccagttcagccctgttggagaaaataagagaagtggtgaagtctatagactctttgatgttggtggccagagaaatgagagaagaaaatggatccatctttttgaaggagttacggctgtaatattctgttctgcaattagcgagtatgatcagacactttatgaggatgaaaacaagaacagaatgatggagactaaggaactttttgagtgggtcctaaggcaaccatgttttgagaaaacatccttcatgttatttttaaacaagtttgacatatttgaaaagaaggtcctgaatgttccgctcaatgtatgtgagtggttcaaacatgattaccagccagtttcaacagagaaacaagagattgaacatgcgtacgagtttgtgaagaaaaagtttgaggaattgtatttccagagcactgctcctgactgtgtagatcgcgtgttcaagatctaccaggcgactgcccctgaccagaagcttgtgaagaagaccttcaagcttggtgatgagactttgagacggaggaatccccttgaagctggcttattatgaccatgcccatgcaacagtatgtatgtttaagagggagatgatatttttacattgagaaattaaaaggtcatctgattttgttgggtatattagaggtcaggtatacaacaatataaaatcgatttgttgattttatgtcaaagtaaatcctgggtggataggaaaagcctttctgaatacctacttgatcaccacatccatctttagaaggttttttagttgggctcaaattttcagacatgacattatgctttgtgattatctttttcattgattgaaagtcacataatgatatatttcatatcctttatttaaaaaaaaaaaaaaaaaa

SEQ ID NO: 57 - protein, 385 aa, Glycine max
MGLVCSRSRRFREAHAEENAQDAEIERRIELETKAEKHIQKLLLLGAGESGRSTIFKQIKLLFQTGFNEAELKSYIPVVHANVYQTIKVLQDGSKELAQNDFDSSKYVISNENQDIGQKLSEIGGTLVYPRLTKELAQEIETMWEDAAIQETYARGNELQVPDCAHYFMENLERLSDANYVPTKEDFLYARVRTTGVVEIQFSPVGENKRSGEVYRLFDVGGQRNERRKWIHLFEGVTAVIFCSAISEYDQTLYEDENKNRMMETKELFEWVLRQPCFEKTSFMLFLNKFDIFEKKVLNVPLNVCEWFKHDYQPVSTEKQEIEHAYEFVKKKFEELYFQSTAPDCVDRVFKIYQATAPDQKLVKKTFKLGDETLRRRNPLEAGLL

SEQ ID NO: 58 - DNA, 1740 bp, Lupinus luteus
gccgggagagtggtatatgttcccagttcttccacaaagaacacacaacaaaacacaagacaatacaatacaactgagcttacttggtttctagtattacctaacttcacacttgacgtatcagtgaaatctagtgtcatattccaccacctcttcacagaacccctttgctttttttcatttcttttcagaaaatatgggcttactctgcagcagaaatcgtcgttataatgacgctgatgctgaagaaaacgcgcaggctgcagaaattgaaagaagaatagagttagaaacaaaggctgaaaagcatattcagaaacttctactactaggtgctggagagtcagggaagtctacaatatttaagcagataaaacttttgtttcaaactggctttgacgaggcagagctaaaaagctacttaccggtcattcatgctaacgtttttcagacaataaaattactgcatgatgggtcgaaggagttggctcagaatgatgttgattcttcaaagtatgttatatctgatgaaaacaaggacattggtgaaaaactctcagaaattggaagcaagctggactacccatatctcaccacggagcttgcaaaggaaatagagactctgtgggaggatgctgcaattcaggaaacatatgctcgtggcaatgaactccaagttccaggctgtgcccattattttatggaaaatctgcaaaggctgtctgatgcaaattatgttcccaccaaggaggatgttttatatgcacgagttcgtacaactggtgttgtagagatacagttcagccctgttggtgaaaataagagaagtggcgaagtctatagactctttgatgttggtggccagagaaatgagagaagaaaatggatccatctttttgaaggagtttcggctgtaatattctgtgctgctattagcgagtatgatcaaactcttttcgaggatgaaaacaagaacagaatgaccgagactaaggagctttttgagtggatcctgaagcaaccatgttttgagaaaacatccttcatgttatttttaaacaaatttgacatatttgagaagaagatcctgaaagtcccactcaatgtttgtgagtggttcaaagattaccagccagtttcaacagggaaacaagagattgaacatgcatatgagtttgtgaagaaaaagtttgaggaattatatttccagagcactgctcctgaacgagtcgatcgcgtctttaaggtctaccggactactgcccttgatcagaagctcatcaagaagactttcaaactcgtcgatgagagtttgaggcggaggaatctatttgaagctggtttgctatgatcactgaacagtatatggttaatggcaatattattttacattgaagaagtaatcaggtgatcatattttggatatatgggaagttcaagtatacaacattatttttggaattaaatcaatttgttgattttatgtcaagttaattctgttgagtgggtagatggggaaagacctttatgaagttttcaacatggcatcattatttgttgattaagactaaccaatgatatatttcatatttcatatttcatttctgctattgtgttttattaatgagctgttacccaaggttctgtgatgaatatgaaatactttgctctttttgccataatgaaacttcaatacttcattgttagagcttttttgcatgcttgtttagaagcggccgc

SEQ ID NO: 59 - protein, 384 aa, Lupinus luteus
MGLLCSRNRRYNDADAEENAQAAEIERRIELETKAEKHIQKLLLLGAGESGKSTIFKQIKLLFQTGFDEAELKSYLPVIHANVFQTIKLLHDGSKELAQNDVDSSKYVISDENKDIGEKLSEIGSKLDYPYLTTELAKEIETLWEDAAIQETYARGNELQVPGCAHYFMENLQRLSDANYVPTKEDVLYARVRTTGVVEIQFSPVGENKRSGEVYRLFDVGGQRNERRKWIHLFEGVSAVIFCAAISEYDQTLFEDENKNRMTETKELFEWILKQPCFEKTSFMLFLNKFDIFEKKILKVPLNVCEWFKDYQPVSTGKQEIEHAYEFVKKKFEELYFQSTAPERVDRVFKVYRTTALDQKLIKKTFKLVDESLRRRNLFEAGLL

SEQ ID NO: 60 - DNA, 1617 bp, Lotus japonicus
ttcagaaaatatgggattactatgtagcaaaaatcgccgttataatgatgctgacactgaagaaaatacacagactgcagaaattgaaagaagaatagagttagaaacgaaggctgaaaaacatattcagaaacttcttctactaggagccggagagtcagggaagtctacaatctttaaacagataaaacttttgtttcaaactggctttgacgaggcagagctaaaaagctaccaaccagtcatacatgctaatgtatatcagacaataaaattactgcatgacggagcaaaggagttggcccagaatgatgttgatttttcaaagtatgttatatccgatgaaaacaaggaaattggggaaaagttatcagaaattgggggcaggctggattacccctgtctcaccaaggaactagcactggaaatagagaatttatggaaggatgctgcaattcaggaaacatatgcccgcggtaatgagctccaagttccagattgtacccactatttcatggaaaatctgcacagactgtccgatgcaaattatgttccaacaaaggatgatgttttgtatgcaagagtgcgtaccactggtgttgtagagatccagttcagccctgttggagaaaataagaaaagcggtgaagtctatagactatttgatgtcggcggtcagagaaatgagaggcgaaaatggatccatctgtttgaaggagtttcagctgtaatattctgtgctgcaattagcgagtacgatcaaacactttttgaggatgaaaacaagaacagaatgatggagactaaggaactttttgaatgggtcttgaagcagccatgttttgagaaaacatccttcatgttatttctaaacaagtttgacatatttgagaagaagatcctgaaagtccctcttaatgtttgtgagtggttcaaagattaccagccagtttcaacagggaaacaggagattgagcacgcatatgagtttgtaaagaaaaagtttgaagaatcatatttccagaacactgccccggaccgtgtagatcgcgtcttcaagatctaccggaccactgctcttgatcagaaggttgtgaagaagacattcaagcttgttgatgagacattgagacggcggaatctctttgaggcgggcttgttatgaccaatttaaccatgtgttattataagtgggataaaatatttacattgaaaagaggtgatcagagattttgggtatactagagatcaggtatactaaaatattaaatcgatttgttgattttatttcccaagtaaatcttgctggatgagtggatggagaaaaggcctttcttaaatagttgattttcacatccatcttcaaagggctaattggttgtgcggaagtttcaagattgatatcatgatctatgattatgtttctatagagtaaaagtcactcatgatatgttgtatttcatattcacatttcatattgcttttccatgcccatggttgtgttgttgtacgtgccttgtgtcatgctgtatgaagttctgaattcatatataggatatgttttgataaacaatttaattaccaaagcgtttatcaaataaaaaaaaaaaaaaaaaa

SEQ ID NO: 61 - protein, 384 aa, Lotus japonicus
MGLLCSKNRRYNDADTEENTQTAEIERRIELETKAEKHIQKLLLLGAGESGKSTIFKQIKLLFQTGFDEAELKSYQPVIHANVYQTIKLLHDGAKELAQNDVDFSKYVISDENKEIGEKLSEIGGRLDYPCLTKELALEIENLWKDAAIQETYARGNELQVPDCTHYFMENLHRLSDANYVPTKDDVLYARVRTTGVVEIQFSPVGENKKSGEVYRLFDVGGQRNERRKWIHLFEGVSAVIFCAAISEYDQTLFEDENKNRMMETKELFEWVLKQPCFEKTSFMLFLNKFDIFEKKILKVPLNVCEWFKDYQPVSTGKQEIEHAYEFVKKKFEESYFQNTAPDRVDRVFKIYRTTALDQKVVKKTFKLVDETLRRRNLFEAGLL

SEQ ID NO: 62 - DNA, 26 bp, synthetic
agaagtttgaggagttatattaccag

SEQ ID NO: 63 - DNA, 18 bp, synthetic
aaggccagcctccagtaa

SEQ ID NO: 64 - DNA, 19 bp, synthetic
gacgtactcgggtgagctt

SEQ ID NO: 65 - DNA, 20 bp, synthetic
gagcattccacacgattaat

SEQ ID NO: 66 - DNA, 35 bp, synthetic
ctagctttggagtaaaaagatttgagtgtgcaacc

SEQ ID NO: 67 - DNA, 35 bp, synthetic
tcttttcgctgtttaattgtaacctttgttctcga

SEQ ID NO: 68 - DNA, 1902 bp, Arabidopsis thaliana
gtaagctatctcgtgtcctcctcataagctgaagtctcacagtcactctcatcatcggtgcttgagtctccttctactatcagggaatatcctacttcctgctttctctattctagaatagctgaagcatgtctttcagtattaccttcatctgatcttcaattgccgtgattaagtaaccgtttttctgcgaaagaaagaagattgacaacagccgtttgatggaatgaagcaccagatacacgacaatgcaacacacatattccttggaaaggacaagtaaaagagcaggcagaagaggagtacctgcataaacagatttgaagtcactgatttcctgcaaagaggcatacttgtaaacggagcagatgacaactttcgcactgcgatcgtcatagtcaaattctcatcaggttcctggatcatcagaaaccaaattagatagaacaataaataaagaaggcggaagttcaaagagcaataccttagcaagctcctctggtctcccggcatacatctgcggttgctcctgtggcatacgcgaattcactctttcatcataagcattgcatattctatcaagcgcaccgatcttactagctgtttgcggcatcggacagacgaagacataagacaaaccatccataccaatgctgcgcactcgctcaccgttgaatactatctccatgggcctctcatcctgactagttccttcaaacgctgctgcatctgctcttgcgtataaaattcgtcccccttgataagtcgagcagccaagaggatttggcatggtctctgcccttcgcatcgtcgtcgttcttcgtgggaatggccgggagctttctacgtttccacgggtaaagatcagaagaggaaggttcgccgcggcgttgcatcttcaccgtcgatttcatcgttacagcgacgcggtaattctaggttgcttagttccattctctctctaaattagggactcgaatgaattgttgaacaagatagagatcttctgatccccgtcgaacattattcaaggccaaaaaagcacacgggaatttagagtaccaatacatatcaaaacctaatgggctttgaatggttgcatgtgtgtgtttattctgatatgcaaagcgatcgatagtcttttccatacaatgtaaactgtaaacaacgtaattaagctaacaatacaactcttttcttctctttttttttgtaaacacaaaacaaaattcacataattcatcgttttcctagttcatctgacattttccaaaattcatcttccattagatccctaatacttgttcatattcatattagggtacatgaataaaagttatcattcttgaactactaaattttcatagtttatttttcttcttttcgtttcactttcgaacaaaacactatacgcatggcatttgcaatgaattccacattatatggaataacaccatgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaagaaaatacctaacaaacaaatattatattgtcatacaaaatatttctatatttttaggttaattaggtttatattcctcgacttttcagggcttatataagaaaggtggaggcaaagcacaatcaaaatcggaggcacggaaatactatcatcacccatctccttaggattcctagttcata

SEQ ID NO: 69 - DNA, 2303 bp, Arabidopsis thaliana
  misc_feature (645)..(645): “n” = a, t, c, or g
aagtcgccctcgaaatatacgttgcttttcttttcttatgtgaagaatagcttcttcttctttgtgtctactataaacactaataatctttttttaatatgattatttttatttatttttgggcaaaatgtttttttaatatgatagtattactatatatttaggttgatcggaagttatatcctctctcattggcctatttttgactgttattttctctatccaaatatatctatcaacattgaatcctgccttattttatcgattcccaaaacacagcttaaccaatttggttttatttcagttaagttttcggttcacaaccaatatgttggagtgttactaatcagatagacgggtaaaataaattgtttgcacaacctattcatatagttaaaataagcgaatagagagataattgaaactacagtgaaaattaacaacaaatggaacatacgtccaagtagttgagacttgaggagtagaaagacaacgtaaggagttgacaaacatgtgagccatttgagactaattcacagacaactaaaatccaagagttatactagtaaatgctttggttattttaatatattttcaagtcgtctgcccgttgaaaactggagaaaatgtatatagagtactatncagctcgaagacaaaaatgtagtgactaaaattaaaatgcttaattggtcggataaacaatccaaaataaatgggaaatgagttttgagttgcgatcgtatcttgcattacaagaagcaaattttagggttaaagtttctgtcacataactccgttacacaattcgcttaattttggatatttgacagaaactaattaatatgtaaactttaatatatatattggattttaggcaatttaacaactaattagtggcggacaaatactgattattttccctttatatatatatatatatataattgaatgcttctatatttgttattttgttgtcacaatgtttttttttttaaaaaattaaatttgatatccttcagtccatcactagttaacaacattgccaaaatatactatcttttcgtcgaaaaaaaaactcaccatacaaactaagtgatttttcagtagtttgtgatcaatgcaaaaatgataaaattaatttaatgtttacaaagctgatgttgggtcgaaagacttggacgtcaaggataacatttggttctaaaatatatggacgtgaaagatagcataccatctttgggactgttcggataagaatgtgtgtgcattagttttggaaagtgtttggccactcggatctttttgtgataatctccagactatgaatattgcggaaatataatccatgatcagagaatatttggcgatgcttgatccatgaccgtaattggagccggatatgttatgatttgatctacaactattattacgctagataagccaattaattatggtaggtgatcgtgaatataacttcactgtccgaaaaagaatagtgtatatgttgccctgaaccatctatctcattaggtgctatgaactagtgagttttaagtatagagggtgcatataagtcgtttttccatcttaaatcaaagacatttcttagtatcttctagatatttcattcttttagtaccatataaattagggatttggacaattcacaattatttattttaaaccaaacaaaataattgttctccgataatcagatgtgacttatgtgatatagtacatataaatatacacactgtttaaatttgtctacccaatcggaatcatagaaactttattgtttacccaaacaaaacggtactgaatatcggaactttttttattaaaaaaaaactgtgagagagaaattgaatcaacgtccaagtcacttgatgcaagaaaaaagcgaaaccaattaaattcccgtaaaaacagaacacaaaagaacaggagagttaatgttctaactgacacgtgtccctaccttgccatacactcacacaattaaaatttctaactctgtctcttatccgaaaaataatcatctccaagtgtaataagaaaatcaaaataaaactctcatttcttcttcttcctcgcctataaatacaactccattctctcatctcctacatcacaaaacaaaaacctcacttaaaaaaaaaaaacagaagacagaaaaaaacaaaaaaaaaaagaaaaagaaaaataaacaaattttcttttcttttttttcctctaaagtttctattttgtctattcgtgtttttttttttacttcctgataatgggagcctatgaaa

SEQ ID NO: 70 - DNA, 2012 bp, Arabidopsis thaliana
atggaccatgaaatcatttgcatatgaactgcaatgatacataatccactttgttttgtgggagacatttaccagatttcggtaaattggtattcccccttttatgtgattggtcattgatcattgttagtggccagacatttgaactcccgtttttttgtctataagaattcggaaacatatagtatcctttgaaaacggagaaacaaataacaatgtggacaaactagatataatttcaacacaagactatgggaatgattttacccactaattataatccgatcacaaggtttcaacgaactagttttccagatatcaaccaaatttactttggaattaaactaacttaaaactaattggttgttcgtaaatggtgctttttttttttgcggatgttagtaaagggttttatgtattttatattattagttatctgttttcagtgttatgttgtctcatccataaagtttatatgttttttctttgctctataacttatatatatatatgagtttacagttatatttatacatttcagatactgatcggcattttttttggtaaaaaatatatgcatgaaaaactcaagtgtttcttttttaaggaatttttaaatggtgattatatgaatataatcatatgtatatccgtatatatatgtagccagatagttaattatttgggggatatttgaattattaatgttataatattctttcttttgactcgtctggttaaattaaagaacaaaaaaaacacatacttttactgttttaaaaggttaaattaacataatttattgattacaagtgtcaagtccatgacattgcatgtaggttcgagacttcagagataacggaagagatcgataattgtgatcgtaacatccagatatgtatgtttaattttcatttagatgtggatcagagaagataagtcaaactgtcttcataatttaagacaacctcttttaatattttcccaaaacatgttttatgtaactactttgcttatgtgattgcctgaggatactattattctctgtctttattctcttcacaccacatttaaatagtttaagagcatagaaattaattattttcaaaaaggtgattatatgcatgcaaaatagcacaccatttatgtttatattttcaaattatttaatacatttcaatatttcataagtgtgatttttttttttttgtcaatttcataagtgtgatttgtcatttgtattaaacaattgtatcgcgcagtacaaataaacagtgggagaggtgaaaatgcagttataaaactgtccaataattactaacacatttaaatwatctaaaaagagtgtttcaaaaaaaattcttttgaaataagaaaagtgatagatatttttacgctttcgtctgaaaataaaacaataatagtttattagaaaaatgttatcaccgaaaattattctagtgccactcgctcggatcgaaattcgaaagttatattctttctctttacctaatataaaaatcacaagaaaaatcaatccgaatatatctatcaacatagtatatgcccttacatattgtttctgacttttctctatccgaatttctcgcttcatggtttttttttaacatattctcatttaattttcattactattatataactaaaagatggaaataaaataaagtgtctttgagaatcgaaacgtccatatcagtaagatagtttgtgtgaaggtaaaatctaaaagatttaagttccaaaaacagaaaataatatattacgctaaaaaagaagaaaataattaaatacaaaacagaaaaaaataatatacgacagacacgtgtcacgaagataccctacgctatagacacagctctgttttctcttttctatgcctcaaggctctcttaacttcactgtctcctcttcggataatcctatccttctcttcctataaatacctctccactcttcctcttcctccaccactacaaccaccgcaacaacca

SEQ ID NO: 71 - DNA, 1300 bp, Arabidopsis thaliana
ctgtctttgaaatatctttatctaccacgacagtttataaaatttgaaagggaaatttatttggtattaggtttctaagaacttcaataaagctatattttcaattacttgttattaaatgcaaaaaaaaaatctaaatggatgtttaggtataactgcaacaaaatttatggaaaaaacaagacttgatgaatgtataatctcaagagttattttaagcataaatttaaatctaaattttctatttaaaattatttgatgaatttttctatttttaattatgataaatgcaagattcatagtatctaaatttaatacagtatatgattaggcttttcattaaacaatataaatatttttttatcttgacataatgatgagtaaataatttaccaactaaatacatataatttttgatcaataccattcaaaagaagaaaaaacactttctcttacttatctttgaattaattttgaaatatttcattaagaaaaattaattgtttaaaatattgctttgattaatgtagacactcctttagaaaactttttaaaggttagggaaaaaatgttaccaaaaaaaagttaggaaaaaaatgtaaaaataaagaaaagaatcttcttcccctaatagaatccaaacaaatgtatcgcttaccatcaagttgcacaaacactcaaaacgctttttgatgatttttcttctataaaatctttctcctcaaccatttcaaagccccctcttctctccttctctcccttttctctctctctcgaagatatcgcctttaattccttcttcttcttctgatttcttcaaagattcatttcttagagatctcagcttctgtaagagtaggctagctaccaaaaaaaaagaaatagaaaaaaacattgatgcttaatcagcagttcttggtgtttgagacacatgtctccaataatactcagtgagatctttctctctgggtttatgttaaattccacaatcaggcgcaggacccatcttgttcaatctttctctgttgtcttcctttactggctttactatgtctcatgatctctgaacttatctccagttttatatctatacgcataattacaacaaaaccctagtttcttttcaaatttctcttcttcgtttttcgtcatctgggtcctgtcctgttctgttgtgtcttgttctgttctgtttttaattcccctaaaaacccattttaaaatattttatctctcttctcttaaaaaaaactcttccctgtttttgtttgtgtgttactaaatggaatcgtcgtcgtcg

SEQ ID NO: 72 - DNA, 1010 bp, Arabidopsis thaliana
cttgtagtgtaccacaacttggtttgcaacactatatatactttttgatatataaaaattctattaaactacttaaatatttcgtcatgaggttatatgcattagaaaaaaatatataacaatattgttggaagactggacaattatcgttgaaactatagctatcaccaagatacaactttttgtggaaaatcttggtggcaattagaaactgttcccaatctcgggcatgaaaatcgttattcaaatatttgtcaacaactagtgaatagtgatacagctatatatgtttgctaatttattgaattatttaatgttacgactttacgtaacaattatttaacgtctattcttgtgtacctcacatttcttatcgtcatgtctcatctcttatattattcacagtacacccatctcttctcgctcactgtggaacctgttgtcaattactcgttttgcattttattggttttcccaattactctatcaattatttatcaaaaaaaaaaaaatgaaacatttactctatatattatttccgcaaacacaaattatactcacatcaacatattcaatacatttttctagtaatgtagaacaactttacagtattctccaaaacgaaactctaattcaaaatttacaagcagataagccaaagataatagaacaacaaaacgccaaattctagttaagcacacaatctcaacgtgcactaaaaacgagtggtgtaagtgaaaaatatcgtcgattataaacattatgggaccagtagcatttgttgcaccaatcgaaaacagacaagcacacatatctcctcatttctcatctggcttcttaatcatttctcataaccccacctcattataaataccaccctttgcgtcacacatataaacatcacaaactaaacaataaaccataccataaaaaacatgaaaatcctctcactttcacttctcttgctcttggccgctacggtctcggcatccgttccagggctcatcgaa

SEQ ID NO: 73 - DNA, 1383 bp, Arabidopsis thaliana
atttttgaggaattttagaagttgaacagagtcaatcgaacagacagttgaagagatatggattttctaagattaattgattctctgtctaaagaaaaaaagtattattgaattaaatggaaaaagaaaaaggaaaaaggggatggcttctgctttttgggctgaaggcggcgtgtggccagcgtgctgcgtgcggacagcgagcgaacacacgacggagcagctacgacgaacgggggaccgagtggaccggacgaggatgtggcctaggacgagtgcacaaggctagtggactcggtccccgcgcggtatcccgagtggtccactgtctgcaaacacgattcacatagagcgggcagacgcgggagccgtcctaggtgcaccggaagcaaatccgtcgcctgggtggatttgagtgacacggcccacgtgtagcctcacagctctccgtggtcagatgtgtaaaattatcataatatgtgtttttcaaatagttaaataatatatataggcaagttatatgggtcaataagcagtaaaaaggcttatgacatggtaaaattacttacaccaatatgccttactgtctgatatattttacatgacaacaaagttacaagtacgtcatttaaaaatacaagttacttatcaattgtagtgtatcaagtaaatgacaacaaacctacaaatttgctattttgaaggaacacttaaaaaaatcaataggcaagttatatagtcaataaactgcaagaaggcttatgacatggaaaaattacatacaccaatatgctttattgtccggtatattttacaagacaacaaagttataagtatgtcatttaaaaatacaagttacttatcaattgtcaagtaaatgaaaacaaacctacaaatttgttattttgaaggaacacctaaattatcaaatatagcttgctacgcaaaatgacaacatgcttacaagttattatcatcttaaagttagactcatcttctcaagcataagagctttatggtgcaaaaacaaatataatgacaaggcaaagatacatacatattaagagtatggacagacatttctttaacaaactccatttgtattactccaaaagcaccagaagtttgtcatggctgagtcatgaaatgtatagttcaatcttgcaaagttgcctttccttttgtactgtgttttaacactacaagccatatattgtctgtacgtgcaacaaactatatcaccatgtatcccaagatgcttttttattgctatataaactagcttggtctgtctttgaactcacatcaattagcttaagtttccataagcaagtacaaatagctatggcgagttccgttttctctcggttttctatatacttt

SEQ ID NO: 74 - DNA, 180 bp, Brassica
cgattttaagctaattagttcgaacaaagagtacaacattaattttctaacagacttagatgcacttgcgaacaacatacttgctgaacaccatatgttatgttggcagggtgagaaattaatcacgtgtagatatagaagtagtagacaaatgatataggtttgtgggaatgaattaat

SEQ ID NO: 75 - DNA, 480 bp, Arabidopsis thaliana
atggaatcgtcgtcgtcgggaacaacttcgtcgacgattcaaacgtcgtcaggatcggaggagagtctcatggagcagaggaaacgtaaacggatgctctcaaaccgtgaatctgcaaggagatcaagaatgaagaaacaaaagctcctagacgatctaacggctcaggttaatcatctgaaaaaagagaacacggagatcgtgacaagtgtcagcatcacaacgcaacactacctaaccgttgaagcagagaactctgttctcagagctcagcttgatgaacttaaccacaggctccaatctctcaacgacatcatcgagttcctcgacagtagcaacaacaacaacaacaacaacatgggcatgtgttcgaaccctctggttggtttggagtgtgatgatttcttcgtgaatcagatgaacatgtcttatattatgaaccagcctctcatggcgtcttctgatgctttaatgtattaa

SEQ ID NO: 76 - protein, 159 aa, Arabidopsis thaliana
MESSSSGTTSSTIQTSSGSEESLMEQRKRKRMLSNRESARRSRMKKQKLLDDLTAQVNHLKKENTEIVTSVSITTQHYLTVEAENSVLRAQLDELNHRLQSLNDIIEFLDSSNNNNNNNMGMCSNPLVGLECDDFFVNQMNMSYIMNQPLMASSDALMY

SEQ ID NO: 77 - DNA, 36 bp, synthetic
gttaattaactcaatcatgaaccttcttctcttcta

SEQ ID NO: 78 - DNA, 39 bp, synthetic
gggcgcgccgaagtttaattcttctaaccactccactat

(19) United States
(12) Patent Application Publication
(10) Pub. No.: US 2004/0187176 A1
(43) Pub. Date: Sep. 23, 2004

(54) Methods for improving plant agronomical traits by altering the expression or activity of plant G-protein alpha and beta subunits

(76) Inventors: Douglas Boyes, Chapel Hill, NC (US); Keith Davis, Durham, NC (US); Alan Jones, Chapel Hill, NC (US); Hemayet Ullah, Chapel Hill, NC (US); Jin-Gui Chen, Chapel Hill, NC (US); Rao Mulpuri, Apex, NC (US); Ani Chatterjee, San Francisco, CA (US); Mary P. Ward, Gaithersburg, MD (US)

Correspondence Address: JENKINS & WILSON, PA, 3100 TOWER BLVD, SUITE 1400, DURHAM, NC, 27707, US

(21) Appl. No.: 10/602,898

(22) Filed: Jun. 24, 2003

Related U.S. Application Data
(60) Provisional application No. 60/392,730, filed on Jun. 28, 2002.
(60) Provisional application No. 60/445,208, filed on Feb. 5, 2003.

(51) Int. Cl. A01H 1/00; C12N 15/82

(52) U.S. Cl. 800/287

(57) ABSTRACT

The invention provides methods for improving plant agronomic traits by altering the expression or activity of plant G-protein alpha and beta subunits that are GPA1 or AGB1 orthologs. The invention also provides such transgenic plants with improved agronomic traits. One embodiment of the invention includes methods for modulating the expression or activity of a plant G-protein beta subunit that is an AGB1 ortholog to alter one or more of the following: the time to reach and duration of flowering, fruit yield, root biomass, seed size, seed shape, plant size, and the number of stem branches. The present invention also encompasses methods for modulating the expression or activity of a plant G-protein alpha subunit that is a GPA1 ortholog to alter one or more of the following: the duration of flowering, fruit and seed yield, plant size, seed size, and seed shape. The compositions of the invention include transgenic plants, and seed thereof, particularly transgenic plants that are dicots, members of the genus Brassica, trees, or gymnosperms.

### REFERENCE TO RELATED APPLICATIONS

This application claims the benefit of and priority to U.S. Provisional Application No. 60/392,730, filed on Jun. 28, 2002, and U.S. Provisional Application No. 60/445,208 filed on Feb. 5, 2003, which applications are herein incorporated by reference in their entirety.

### FIELD OF THE INVENTION

The invention relates to the genetic manipulation of plants, particularly to alteration of the expression or activity of the plant G-protein subunits, Gα and Gβ.

### BACKGROUND OF THE INVENTION

Heterotrimeric G-proteins are key signal transduction components that couple the perception of an external signal by a G-protein coupled receptor (GPCR) to downstream effectors. The G-protein complex is comprised of Gα, Gβ and Gγ monomeric subunits that assemble as a heterotrimer that physically associates with a GPCR. Activation of the GPCR triggers the Gα subunit to exchange GDP for GTP, thus activating the G-protein. Once active the heterotrimeric complex dissociates from the GPCR and the Gα subunit separates from the Gαγ heterodimer. Both GTP-bound Gα and the Gαγ heterodimer transduce the signal to downstream effectors.

Heterotrimeric G-proteins have been studied extensively in animals. To date, 23 Gα, 6 Gβ, and 11 Gγ genes have been reported in mammals (Vanderbeld and Kelly (2000) Biochem. Cell Biol. 78: 537-550). The alpha subunits are classified into four subfamilies: Gs, Gi, Gq, and G₁₂. In contrast, relatively little is known about the role G-proteins play in plants. While multiple genes encode each of the Gα, Gβ and Gγ subunits in animals, sequence similarity searches suggest the Arabidopsis genome sequence contains one Gα (GPA1), one Gβ (AGB1) and two Gγ genes. GPA1 shares 36% amino acid sequence identity to mammalian Gα subunits (Ma et al. (1990) Biochemistry 87: 3821-3825). Similarly, AGB1 shares greater than 41% amino acid sequence identity to animal Gβ subunits (Weiss et al. (1990) Plant Biology 91: 9554-9558).

The lack of structural redundancy in the Arabidopsis genome facilitates examination of the function of the G-protein α and β subunits through the generation of loss-of-function mutants. Loss-of-function mutants in the Gα subunits of rice and Arabidopsis are completely viable, but show several developmental defects. The rice mutant exhibits shortened internodes, rounded seeds, and partial insensitivity to gibberellin, whereas the Arabidopsis mutants have rounded leaves and altered sensitivity to a number of phytohormones (Ashikari et al. (1999) Proc. Natl. Acad. Sci. 96: 10284-10289; Fujisawa et al. (1999) Proc. Natl. Acad. Sci. 96:7575-7580; Ueguchi-Tanaka et al. (2000) Proc. Natl. Acad. Sci. 97: 11638-11643; Wang et al. (2001) Science 292: 2070-2072; (Ullah et al. (2001) Science 292: 2066-2069). A loss-of-function mutant in the Gβ subunit of Arabidopsis (AGB1) exhibits several defects including short, blunt fruits, rounded leaves, and shortened floral buds (Lease et al. (2001) Plant Cell 13: 2631-2641).

Transgene expression from a constitutive promoter is widely used in functional genomic studies. However, the generation of stable transgenic lines in which a gene required for normal growth and development has been inactivated is often impossible due to the resulting deleterious phenotype. The estimate for the number of essential genes is not known precisely, is believed to represent a significant proportion of the genome. More than 500 genes in Arabidopsis may be essential for proper embryogenesis alone (Frazmann et al. (1995) Plant J.7: 341-350). Other estimates suggest that about 3500-4000 genes are predicted to be essential based on the frequency of fusca mutants in large-scale seed colour and seedling-lethal (Misera et al. (1994) Mol. Gen. Genet 244:242-252). Recently, Budziszewski et al. identified more than 500 seedling lethal mutants from screening about 38,000 insertional mutant lines (Budziszewski et al. (2001) Genetics 159:1765-1778).

Aside from the inability to recover transgenic lines when the resulting phenotype is deleterious, researchers also face the problem of dissecting the pleiotropic phenotypes that often result from ectopic expression or down-regulation of non-essential genes. Two methods are widely used to circumvent the problems encountered with ubiquitous transgene expression. The first is to drive expression of a transgene from an inducible promoter regulated by heat shock or the application of chemicals such as dexamethasone or anhydrotetracycline (Aoyama, T., & Chua, N. H. (1997) Plant J. 11:605-612; Ulmasov et al (1997) Plant Mol Biol. 35:417-24). However, the main disadvantage of such promoters is that the application of heat shock or chemicals themselves can be deleterious (Kang et al. (1999) Plant J. 20:127-33; Peterson, N. S. (1990) Adv. Genet. 28:275-296). In addition, inducible expression from such promoters is ectopic and often leaky.

A second alternative to overcome the problems associated with constitutive transgene expression is the use of tissue specific promoters to confine transgene expression to specific tissues or cell types. This approach is dependent on the availability of well-characterized promoters that can be used to provide the desired temporal and spatial pattern of expression. Even if a suitable promoter is available, position-effect variation in promoter expression pattern and activity level often requires the analysis of many independent lines to define a consistent transgenic phenotype. As with constitutive transgene expression, if the gene to be suppressed is essential, it is very difficult to generate stable transgenic lines. Driving the expression of essential genes in specific tissues would be a powerful alternative to elucidate their direct function. The current use of tissue specific promoters requires custom vector design and construction and has not been optimized for high-throughput gene function analysis.

To overcome the foregoing problems in Drosophila melanogaster, Brand and Perrimon utilized the yeast bipartite Gal4 transactivating system driven by tissue-preferred promoters or trapped enhancers (Brand, A. H. and Perrimon, N. (1993) Development 118:401-415). In this approach, the target gene (UAS-effector) is separated from transcriptional activation elements (GAL4 transactivator) by maintaining the two constructs in separate transgenic fly lines. Target genes remain silent in the absence of its activator, and in the activator line, the activator protein is present but has no target gene to activate. Down-regulation of essential genes, therefore, will not be counter-selected by this approach, as the target genes are silent during the transformation and regeneration processes and are only activated upon crossing with the GAL4 transactivator line. Thus, effects of the suppression or ectopic expression of genes of interest will be observed under otherwise normal condition.

Recently, a Gal4-UAS transactivating system has been established for Xenopus (Hartley et al. (2002) Proc. Natl. Acad. Sci. USA 99:1377-1382). Guyer et al. demonstrated the concept in Arabidopsis and Molina et al. put the system to practice by co-suppressing protoporphyrinogen oxidase expression via transactivation (Guyer et al. (1998) Genetics 149:633-639; Molina et al. (1999) Plant J. 17:667-678). However, to date transactivation in plants is based on either constitutive or inducible expression by chemical application (Aoyama and Chua (1997) Plant J. 11:605-612; Guyer et al., supra; Schwechheimer et al. (1998) Plant Molecular Biology 36 :195-204; Molina et al., supra). Tissue- and/or stage-preferred gene expression or silencing by transactivation system to high-throughput functional approaches has heretofore not been established. In particular, the advantage of a transactivating system in plants to circumvent lethality associated with essential gene silencing has not yet been realized.

### SUMMARY OF THE INVENTION

The present inventors have discovered previously unobserved developmental and phenotypic abnormalities resulting from altered expression or activity of the Gα (GPA1) and Gβ (AGB1) subunits of Arabidopsis. Many of the traits exhibited by the Arabidopsis mutants are desired characteristics in agriculturally important plant species. This unexpected discovery has facilitated the development of methods for the generation of plants having improved agronomical traits.

In a general aspect, therefore, the invention provides methods and compositions for improving plant agronomic traits. In one embodiment, the invention provides methods for altering one or more of the following plant traits: time to flowering; duration of flowering; fruit yield; root biomass; seed size; seed shape; number of stem branches; and plant size. The methods comprise introducing into a plant cell an expression cassette comprising a nucleotide sequence that is antisense, sense, dsRNA, a ribozyme, an inverted repeat to a plant nucleotide sequence that is AGB1 or an AGB1 ortholog; a nucleotide sequence that is GPA1 or a GPA1 ortholog; or causing a disruption in a gene in a plant cell other than Arabidopsis, wherein the gene is an AGB1 ortholog endogenous to the plant cell; and regenerating a plant that has a stably integrated expression cassette or disrupted gene from the plant cell wherein the plant exhibits one or more of the above listed traits.

Another embodiment of the present invention encompasses methods for altering one or more of the following traits: duration of flowering; fruit and seed yield; plant size; and seed size and shape. The methods comprise introducing into a plant cell an expression cassette comprising a nucleotide sequence that is antisense, sense, sense containing a dominant site-directed mutation, dsRNA, a ribozyme, an inverted repeat to a nucleotide sequence that is GPA1 or a GPA1 ortholog; or causing a disruption in a gene in a plant cell that is not Arabidopsis thaliana or Oryza sativa, wherein the gene is a GPA1 ortholog endogenous to the plant cell; and regenerating a plant that has a stably integrated expression cassette or disrupted gene from the plant cell wherein the plant exhibits one or more of the above listed traits.

The compositions of the invention include transgenic plants having stably integrated into their genome an expression cassette comprising a nucleotide sequence that is antisense, sense, dsRNA, a ribozyme, or an inverted repeat to a nucleotide sequence that is AGB1 or an AGB1 ortholog. Further included are transgenic plants that have a disruption in a gene that is an AGB1 ortholog endogenous to the plant. Other embodiments include transgenic plants having stably integrated into their genome an expression cassette comprising a nucleotide sequence that is antisense, sense, sense containing a dominant site-directed mutation, dsRNA, a ribozyme, or an inverted repeat of GPA1 or an GPA1 ortholog. In addition, the invention includes transgenic plants that have a disruption in a gene that is a GPA1 ortholog endogenous to the plant.

In particular embodiment, the invention provides transgenic plants that have increased root biomass and methods for generating these transgenic plants. The compositions of the invention include transgenic plants, and seed thereof, each comprising separate driver cassettes for root-preferred expression of a synthetic chimeric transcription factor and target cassettes for the transcription factor driven antisense expression of at least a portion of an AGB1 gene sequence, or an ortholog thereof. Promoters of the invention include root-preferred promoters such as, but not limited to, D2, D3, D4, D6, D11, and D19 promoters and bZIP root-preferred promoters such as D5 bZIP promoter. The transgenic plants of the invention are monocots, dicots, vegetable crops, tomato, potato, pea, spinach, tobacco, soybean, sunflower, peanut, alfalfa, mint, cotton, rice, maize, oats, wheat, barley, sorghum, grasses, Brassica, Brassica napus, and Arabidopsis.

The compositions of the invention are transgenic plants, and seed thereof, having increased root biomass, the plants comprising, stably integrated in their genome, a driver cassette comprising a synthetic chimeric transcription factor open reading frame operably linked to a root-preferred promoter; and a target cassette comprising at least a portion of an AGB1 gene sequence set forth in SEQ ID NO:1, or an ortholog thereof, in the antisense orientation operably linked to a minimal promoter operably linked to at least one cognate upstream activating sequence.

The methods of the invention are directed to methods for producing transgenic plants having increased root biomass comprising generating a transgenic plant comprising a driver cassette comprising a synthetic chimeric transcription factor open reading frame operably linked to a root-preferred promoter and a target cassette comprising at least a portion of an AGB1 gene sequence set forth in SEQ ID NO:1, or an ortholog thereof, in the antisense orientation operably linked to a minimal promoter operably linked to at least one cognate upstream activating sequence, wherein each of the driver and the target cassettes is stably integrated in the genome of the plant and the plant has an increased root biomass.

Advantageously, the present methods achieve the uncoupling of phenotypic traits in transgenic plants, where one or more traits are desirable while others are deleterious to plant growth or yield. For example, transgenic plants of the invention have increased root biomass, while displaying an otherwise normal phenotype. The plants with increased root biomass are a result of root-preferred antisense expression. In addition, the root-preferred expression in the transgenic plants of the invention eliminates the problem of positional effects and transgene copy number.

It is thus an object of the invention to provide methods for improving plant agronomic traits. It is an additional object of the invention to provide transgenic plants having improved agronomic traits, where the traits include one or more of the following: time to flowering; duration of flowering; fruit yield; root biomass; seed size; seed shape; number of stem branches; and plant size.

An object of the invention having been stated hereinabove, and which is addressed in whole or in part by the present invention, other objects will become evident as the description proceeds when taken in connection with the accompanying drawings as best described hereinbelow.

### BRIEF DESCRIPTION OF THE DRAWINGS

FIG. 1 is a schematic diagram of data taken from Table 2 depicting the developmental progression of WS control versus gpa1-2 and gpa1-1, and CoI control versus agb1-2 and agb1-1 mutant Arabidopsis thaliana plants.

FIG. 2 shows representative images of mature root phenotypes for G-protein alpha and beta mutant transgenic plants. CoI control, agb1-1 and agb1-2 (FIG. 2A), and Ws control, gpa1-1 and gpa1-2 (FIG. 2B) plants were grown in short days (8:16 light:dark) for 3 weeks and then transferred to long days (16:8 light:dark) for two weeks.

FIG. 3. FIG. 3 shows relative expression of transcripts in the transgenic and vector lines as detected by Real Time PCR. The PCR cycle number at which the fluorescence from the PCR products reached 30 was taken as the Cₜ (Cycle Threshold) value for the corresponding reaction. The primers used were designed to amplify a fragment from the coding sequence of AGB1 or GPA1 with RNA from 10-day old seedlings.

FIGS. 4A and 4B are graphical representations of quantified lateral root primordia in transgenic plants with altered expression or activity of G-protein protein alpha and beta subunits. FIG. 4A shows the results for transgenic seedlings transferred to plates with or without auxin and grown for 96 hours. The standard error of the mean is based on 10 seedlings. The agb1-2 (AGB1) genotype is a genetically complemented agb1-2 mutant. FIG. 4B shows the results for transgenic seedlings transferred to plates with or without auxin and/or dexamethasone. The standard error of the mean is based on 10 seedlings. The GOX and BOX genotypes are transgenic lines that over-express GPA1 and AGB1, respectively, and the GPA1* genotype are lines that expresses a mutated GPA1 protein that is constitutively active.

FIGS. 5A and 5B illustrate a transactivation scheme for tissue-preferred gene expression. In FIG. 5A, driver lines are expressing the yeast GAL4 DNA binding domain fused to the transcriptional activation domain of herpes simplex virus 2XF-VP16 protein (DBD). The indicated promoters are fused upstream from the DBD. Target lines contain four repeat concatamers of the yeast consensus binding site for Gal4 (UAS), linked to the 35S minimal promoter and the gene of interest in sense or antisense orientation. Homozygous driver lines were crossed to hemizygous (primary transformant-T1) target lines to activate latent transgenes.

FIG. 5B is a schematic diagram of the promoters used in each of the transgenic driver plant lines. The letter “D” is used to designate the transgenic driver plant lines. PG91 is a transgenic driver plant line having a ubiquitous promoter providing constitutive expression. Each of the promoters are named, and the predominant expression location noted, according to the original reference for the associated gene.

FIG. 6 is a table of experimentally determined expression patterns for the seven transgenic driver plants of the invention based on GUS target gene activity. Hygromycin selected T1 hemizygous driver lines were crossed to homozygous GUS-UAS target lines. Basta selected F2 progeny lines from the respective crosses were analyzed for GUS reporter gene activity. A line homozygous for the UAS-GUS target construct without a driver (pPG340) was used as a negative control. The expression pattern produced by each driver, designated as described in FIG. 5, is listed in column two.

FIGS. 7A, 7B, 7C and 7D illustrate the results of an experiment in which driver plant line D5 was crossed with a target plant having target AGB1 gene antisense sequence (AGB1.as) to obtain target cassette expression in root tissue. The experiment demonstrates the ability of a tissue-preferred driver to separate pleiotropic phenotypes, selecting for only the desirable agronomic trait.

FIGS. 7A and 7B are photographs of control seedlings transformed with only the target line AGB1.as and seedlings resulting from the D5X AGB1.as cross, respectively. The expression of AGB1 in antisense orientation in the root resulted in more lateral root production (lower panel B) as compared to the control plant, which is transformed with only the AGB1.as target transgene (upper panel A).

FIG. 7C is a graphical representation of the quantification of number of lateral roots of the seedlings depicted in panels A & B. The seedlings were cleared in chloral hydrate and number of lateral root primordia counted for 10 seedlings. Inset shows D5 driven GUS expression in the lateral root of early stage seedling.

FIG. 7D is a pictorial representation of control plant (left panel), plant from the D5X AGB1.as cross (middle panel) and plant from PG91X AGB1.as (right panel). In contrast to the constitutively active PG91X AGB1.as plant (AGB1 knock out) that is smaller and has rounded and crinkled leaves, the root-preferred D5/AGB1.as plant has an almost identical size and leaf shape to that of the control plant.

FIG. 8 is a schematic representation of a driver plasmid of the invention. Features represented in black are derived from pGPTV-HYG (Becker et al. (1992) Plant Mol. Biol. 20:1195-1197) and include: oriV, origin of replication; Kan^{r}, bacterial kanamycin resistance gene cassette; LB, left border of T-DNA; RB, right border of T-DNA; Pₙₒₛ, Agrobacterium nopaline synthase promoter; Hyg^{r}, HptII open reading frame conferring plant hygromycin resistance; Term., g7 transcriptional terminator. Features represented in gray are as described in Schwechheimer et al. (1998) Plant Molecular Biology 36:195-204 and include: Gal4 DBD, GAL4 DNA binding domain; 2xVP16 AD, doubled VP16 transcriptional activation domain; Term., transcriptional terminator. The hatched box represents the promoter used to drive expression of the Gal4DBD-2XVP16AD fusion protein. The plasmid is not drawn to scale.

### DETAILED DESCRIPTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All patents and publications mentioned herein are incorporated herein by reference for the purpose of describing and disclosing, for example, the cell lines, constructs, and methodologies that are described in the patents and publications, which might be used in connection with the presently described invention. The patents and publications discussed throughout the text are provided solely for their disclosure prior to the filing date of the present application. Nothing herein is to be construed as an admission that the inventors are not entitled to antedate such disclosure by virtue of prior invention.

As used herein and in the appended statements of the invention, the singular forms “a”, “and”, and “the” include plural referents unless the context clearly dictates otherwise. Thus, for example, reference to “a construct” includes a plurality of such constructs, and so forth.

Definitions

While the following terms are believed to be well understood by one of ordinary skill in the art, the following definitions are set forth to facilitate explanation of the invention.

“Antisense DNA nucleotide sequence” is intended to mean a sequence that is in inverse orientation to the 5′ to 3′ native orientation of that nucleotide sequence. The antisense nucleotide sequence encodes an RNA transcript that is complementary to and capable of hybridizing to the endogenous messenger RNA (mRNA) produced by transcription of the DNA nucleotide sequence for the native gene.

“Antisense orientation” is intended to mean a nucleotide sequence that is in inverse orientation to the 5′ to 3′ native orientation of the nucleotide sequence or gene. The nucleotide sequence in antisense orientation encodes an RNA transcript that is complementary to and capable of hybridizing to the endogenous messenger RNA (mRNA) produced by transcription of the DNA nucleotide sequence for the native gene. It is understood that the antisense nucleotides of the invention need not be completely complementary to the target sequence, gene, RNA or ortholog thereof, nor that they hybridize to each other along their entire length to modulate expression or to form specific hybrids. Furthermore, the antisense nucleotides of the invention need not be full length with respect to the target gene or RNA. In general, greater homology can compensate for shorter polynucleotide length.

The phrase “at least a portion of a gene sequence” is intended to mean a nucleotide sequence that consists of at least 8 consecutive nucleotides of the gene sequence up to as much as one less than the complete number of consecutive nucleotides of the gene sequence. For example, at least a portion of a gene sequence is at least 8, 10, 12, 14, 15, 16, 17, 18, 19, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 800, 825, 850, 875, 900, 925, 950, 975, or at least 1000 consecutive nucleotides of the gene sequence.

A “bZIP root-preferred promoter” is used herein to refer to a nucleotide sequence that promotes root-preferred RNA transcript expression of a bZIP transcription factor open reading frame in a plant. A bZIP transcription factor is a protein belonging to the evolutionary class of basic domain/leucine zipper (bZIP) transcription factor proteins as described in Alber (1992) Curr Op Gen Devel 2:205-210 and Pabo & Sauer (1992) Annu Rev Biochem 61:1053-1095, herein incorporated by reference in their entirety. One example of a bZIP root-preferred promoter is the D5 bZIP promoter (SEQ ID NO:71) described herein. Other examples of bZIP root-preferred promoters of the invention are promoters that direct root-preferred expression in plants of orthologs of the Arabidopsis ATB2 gene (SEQ ID NO:75). In one case the bZIP root-preferred promoters of the invention direct root-preferred RNA transcript expression in a dicot plant. In another case the bZIP root-preferred promoters of the invention direct root-preferred RNA transcript expression in dicot and/or monocot plants.

The phrase “causing a disruption in a gene” is used herein to refer to a means of altering the expression of a gene. Examples of methods for causing a disruption in a target plant gene (e.g., a GPA1 or AGB1 ortholog) include the use of ribozymes, random mutagenesis of a target gene using chemicals, irradiation, T-DNA or transposon insertion, expression of a sense sequence containing a dominant site-directed and alteration of expression of target gene accessory proteins.

The phrase “cognate upstream activating sequence” herein refers to a nucleotide sequence comprising a binding site for a synthetic chimeric transcription factor of the invention having a DNA binding specificity that is not found in plants. In the invention, binding of the synthetic chimeric transcription factor in a plant to the cognate upstream activating sequence drives transcription of a target gene sequence operably linked to a minimal promoter operably linked to the cognate upstream activating sequence. The compositions and methods of the invention include the use of 1, 2, 3, 4, 5, 6, 7, 8 or more cognate upstream activating sequences. The cognate upstream activating sequences of the invention are, in some cases, consensus or optimized sequences. Examples of the cognate upstream activating sequences of the invention include, but are not limited to, the GAL4 upstream activating sequences of the invention; LexA upstream activating sequences described, for example, in Schwechheimer et al. (1998) Plant Molecular Biology 36:195-204; 434 upstream activating sequences (operators) described, for example, in Wilde et al. (1994) Plant Molecular Biology 24:381-388; and LacI^{his} upstream activating sequences (pOp lac operators) described, for example, in Moore et al. (1998) PNAS 95:376-381.

“D5 bZIP promoter” herein refers to a nucleotide sequence set forth in SEQ ID NO:71.

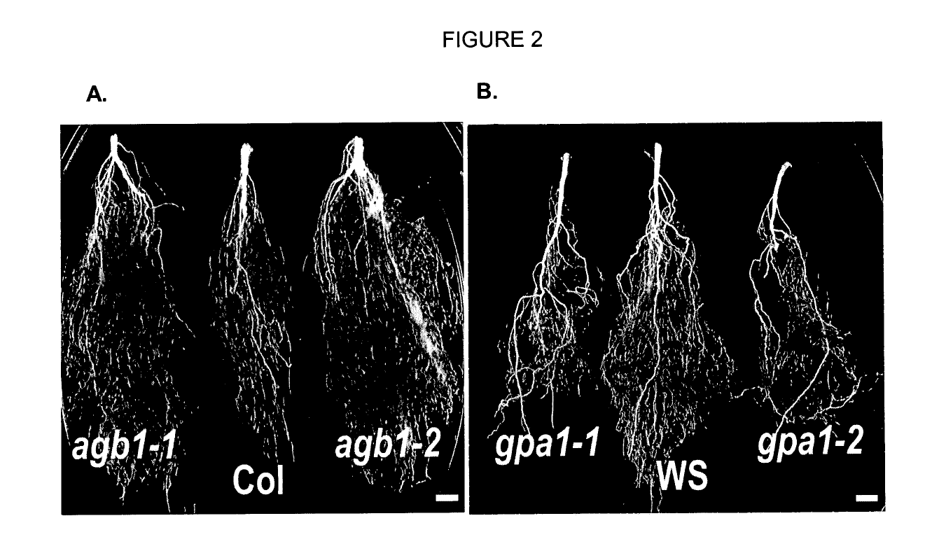

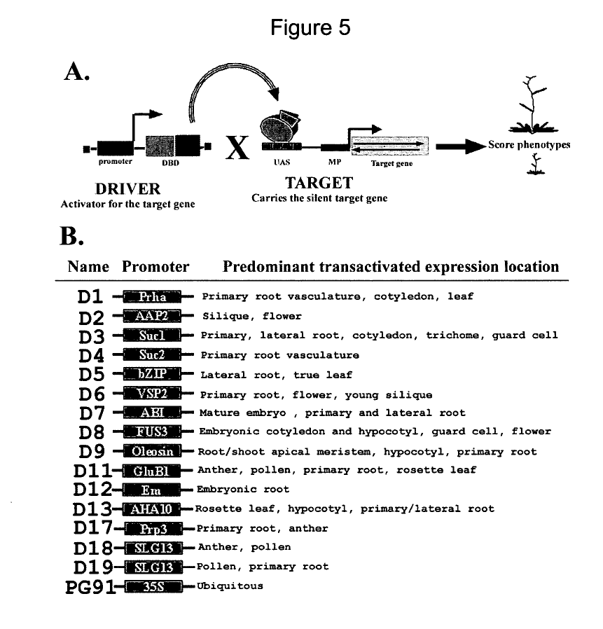

A “driver cassette” is intended to mean a recombinant nucleotide expression cassette comprising a synthetic chimeric transcription factor open reading frame functionally linked to a promoter of the invention. One example of a driver cassette of the invention is depicted in FIG. 5-2 and comprises the Promoter, GAL4 DBD, 2XVP16 AD, and Term, therein, described in Schwechheimer et al. (1998) Plant Molecular Biology 36:195-204, herein incorporated by reference in its entirety. In the example, the Promoter is a promoter of the invention and is located at a position that replaces the original 2X 35S promoter sequence described by Schwechheimer et al. (1998).

The term “dsRNA,” as used herein, refers to RNA hybrids comprising two strands of RNA. The dsRNAs of the invention may be linear or circular in structure. The hybridizing RNAs may be substantially or completely complementary. By “substantially complementary,” it is meant that when the two hybridizing RNAs are optimally aligned using the alignment programs as described above, the hybridizing portions are at least 95% complementary.

The recombinant “expression cassettes” of the invention contain 5′ and 3′ regulatory sequences necessary for transcription and termination of the polynucleotide of interest. Expression cassettes generally comprise at least one promoter and a transcriptional terminator. Promoters of the present invention are described more fully herein. In certain embodiments of the invention, other functional sequences are included in the expression cassettes. Such functional sequences include, but are not limited to, introns, enhancers, and translational initiation and termination sites and polyadenylation sites. The control sequences function in at least one plant, plant cell, or plant tissue. These sequences may be derived from one or more genes, or can be created using recombinant technology. Polyadenlation signals include, but are not limited to, the Agrobacterium octopine synthase signal (Gielen et al (1984) EMBO J. 3:835-846) and the nopaline synthase signal (Depicker et al. (1982) Mol. and Appl. Genet. 1:561-573). Transcriptional termination regions include, but are not limited to, the terminators of the A. tumefaciens Ti plasmid octopine synthase and nopaline synthase genes. (Ballas et al. (1989) Nuc. Acid Res. 17:7891-7903; Guerineau et al. (1991) Mol. Gen. Genet. 262:14144; Joshi et al. (1987) Nuc. Acid Res. 15:9627-9639; Mogen et al. (1990) Plant Cell 2:1261272; Munroe et al. (1990) Gene 91:15158; Proudfoot (1991) Cell 64:671-674; and Sanfacon et al. (1991) Genes Devel. 5:14149).

A “GAL4/VP16 open reading frame” is, for example, a GAL4 DNA binding domain open reading frame fused to at least one VP16 transcriptional activation domain open reading frame. A GAL4/VP16 open reading frame is, for example, a GAL4 DNA binding domain open reading frame fused to 1, 2, 3, 4, 5, 6, 7 or 8 or more copies of the VP16 transcriptional activation domain such as that described in Schwechheimer et al. (1998) Plant Molecular Biology 36:195-204, herein incorporated by reference in its entirety.

The phrase “GAL4 upstream activating sequence,” also used interchangeably with “GAL4 UAS,” is used herein to refer to a nucleotide sequence comprising a binding site for a GAL4/VP16 transcription factor DNA binding domain. In the invention, binding of the GAL4/VP16 transcription factor to the upstream activating sequence in a plant drives transcription of a target gene sequence operably linked to a minimal promoter operably linked to the GAL4 upstream activating sequence. GAL4 upstream activating sequences are known to one of skill in the art, see for example, Schwechheimer et al. (1998) Plant Molecular Biology 36:195-204, herein incorporated by reference in its entirety. The compositions and methods of the invention include the use of “at least one GAL4 upstream activating sequence” as described in Schwechheimer et al. (1998) who demonstrate use of 1-8 consensus GAL4 UAS sequences. Additional references to GAL4 upstream activating sequences useful in the invention are, for example, Fang et al. (1989) Plant Cell 1:141-150; Gill & Ptashne (1988) Nature 334:721-724; Giniger et al. (1985) Cell 40:767-774; Guerineau & Mullineaux (1993) In: Croy RDD (ed) Plant Molecular Biology Lab-fax, pp.125-127, BIOS Scientific Publishers, London; and Jefferson (1987) Plant Mol Biol Rep 5:387-405, herein incorporated by reference in their entirety.

The meaning of the term “gene” as it is used herein does not necessarily require that the entire plant genomic sequence be encompassed. For example, in some cases the term gene is used when referring solely to an open reading frame that encodes a polypeptide. In other cases the term gene is used to refer to a plant nucleotide sequence that includes an open reading frame that encodes a polypeptide and associated promoter elements. In any case the term gene as it is used herein need not require inclusion of all regulatory elements. The manner of use of the term gene is intended to be and consistant with that of one of ordinary skill in the art.

The phrase “introducing a polynucleotide” into a host cell can performed by any means known in the art including transfection, transformation, transduction, electroporation, particle bombardment, infection (bacterial or viral) and the like. The introduced polynucleotide may be maintained in the cell stably if it is integrated into the host chromosome or incorporated into a non-chromosomal autonomous replicon. Alternatively, the introduced polynucleotide may be present on an extra-chromosomal non-replicating vector and be transiently expressed or transiently active.

A phrase “minimal promoter” is used herein as it is used by one of ordinary skill in the art and is a promoter nucleotide sequence that promotes transcription in a plant but lacks intrinsic transcriptional activity. The minimal promoter sequences of the invention comprise the numerous minimal promoters known to those of skill in the art. One example of a minimal promoter of the invention is the CaMV 35S minimal promoter described in Moore et al. (1998) PNAS 95:376-381, herein incorporated by reference in its entirety. Additional examples of minimal promoters, including a NOS minimal promoter, are found in Schwechheimer et al. (1998) Plant Molecular Biology 36:195-204; Wilde et al. (1994) Plant Molecular Biology 24:381-388; and Puente et al. (1996) The EMBO Journal 15:3732-3743, also incorporated herein by reference in their entirety.

As used herein, “nucleic acid” and “polynucleotide” and “nucleotide sequence” are interchangeably and refer to, for example, RNA or DNA that is linear or branched, single or double stranded, or a hybrid thereof. The term also encompasses RNA/DNA hybrids. Less common bases, such as inosine, 5-methylcytosine, 6-methyladenine, hypoxanthine and others are encompassed by the term. Also included by the term are other modifications, such as modifications to the phosphodiester backbone, or the 2-hydroxy in the ribose sugar group of the RNA.

By “operably linked” is meant that a polynucleotide is functionally linked to a promoter, such that the promoter is capable of initiating transcription of the polynucleotide in a plant.

“Orthologs” of the Arabidopsis AGB1, GPA1 and ATB2 genes are nucleotide sequences from other, non-Arabidopsis plant species that encode polypeptides that share substantial sequence conservation with the Arabidopsis AGB1, GPA1 and ATB2 sequences. The phrases “percent sequence conservation” and “percent sequence similarity” are herein used interchangeably. By “substantial sequence conservation” is meant a polypeptide sequence that has at least 70% percent sequence conservation, preferably at least 80%, 81%, 82%, 83%, 84%, 85%, 86%, 87%, 88%, 89% 90%, 91%, 92%, 93%, 94%, 95%, 96%, 97%, 98%, or 99% percent sequence conservation to the gene product of sequence that it is orthologous to. For the purposes of the invention, the “percent sequence conservation” or “percent sequence similarity” between two polypeptide sequences is determined according to either the BLAST program (Basic Local Alignment Search Tool) (Altschul, S. F., W. Gish, et al. (1990) J. Mol. Biol. 215: 403-10 (PMID: 2231712)) at the National Center for Biotechnology, or the Smith Waterman Alignment (Smith, T. F. and M. S. Waterman (1981) J. Mol. Biol. 147: 195-7 (PMID: 7265238)), as incorporated into GENEMATCHER PLUS™. One of skill in the art will recognize that these values can be determined by taking into account codon degeneracy, amino acid similarity, reading frame positioning, and the like.

Thus, the phrase “a GPA1 or AGB1 ortholog” is referring to a gene from a species of plant other than Arabidopsis whose gene product shares substantial sequence conservation to GPA1 or AGB1. An “ortholog of an AGB1 gene sequence” refers to a gene from a species of plant other than Arabidopsis that shares substantial sequence conservation to AGB1 set forth in SEQ ID NO:1. An ortholog of the Arabidopsis ATB2 gene sequence set forth in SEQ ID NO:75 refers to a gene from a species of plant other than Arabidopsis whose gene product shares substantial sequence conservation to ATB2 and the ATB2 gene product set forth in SEQ ID NO:76.

The term “ribozyme,” as used herein, means a catalytic RNA-based enzyme capable of targeting and cleaving particular base sequences in both DNA and RNA. Ribozymes comprise a polynucleotide sequence that is complementary to a portion of a target nucleic acid and a catalytic region that cleaves the target nucleic acid. Ribozymes can be designed to specifically pair with and inactivate a target RNA by catalytically cleaving the RNA at a targeted phosphodiester bond. Ribozymes can be designed to bind to exons, introns, exon-intron boundaries and control regions, such as the translational initiation sites. In the methods of the invention ribozymes are used to reduce the expression of a target gene or RNA that is AGB1, GPA1 or an ortholog thereof.

“Root-preferred expression” is used herein to mean RNA transcript expression at greater levels in root tissue of a plant than in other tissues of the plant.

A “root-preferred promoter” is a nucleotide sequence that promotes root-preferred RNA transcript expression in a plant. For example, a root-preferred promoter is a nucleotide sequence that promotes root-preferred RNA transcript expression in a dicot plant. Other examples of root-preferred promoters include D2, D3, D4, D5, D6, D11, and D19.

“Root-preferred RNA transcript expression” is used herein to mean RNA transcript expression at greater levels in a plant root tissue than in other tissues of the plant.

The phrase “synthetic chimeric transcription factor open reading frame” is, for example, a GAL4/VP16 open reading frame of the invention. The synthetic chimeric transcription factors of the invention also include, but are not limited to, the chimeric transcription factors, and functional combinations thereof, described in Moore et al. (1998) PNAS 95:376-381; Schwechheimer et al. (1998) Plant Molecular Biology 36:195-204; and Wilde et al. (1994) Plant Molecular Biology 24:381-388, herein incorporated by reference in their entirety. In the invention, a synthetic chimeric transcription factor is, for example, a GAL4 DNA binding domain fused to 1, 2, 3, 4, 5, 6, 7 or 8 or more copies of a VP16 or a THM18 transcriptional activation domain. A synthetic chimeric transcription factor of the invention is also, for example, a LexA DNA binding domain fused to 1, 2, 3, 4, 5, 6, 7 or 8 or more copies of a VP16 or a THM18 transcriptional activation domain. Other examples of synthetic chimeric transcription factors of the invention include a 434 DNA binding domain fused to 1, 2, 3, 4, 5, 6, 7 or 8 or more copies of a VP16 or a THM18 transcriptional activation domain. Another example of a synthetic chimeric transcription factor of the invention includes a LacI^{his} DNA binding domain fused to 1, 2, 3, 4, 5, 6, 7 or 8 or more copies of a Gal4 transcriptional activation domain II.

A “target cassette” is intended to mean a recombinant nucleotide expression cassette comprising at least a portion of a target gene sequence functionally linked to a minimal promoter of the invention functionally linked to a cognate upstream activating sequence.

For the purposes of the invention, “transgenic” refers to any plant, plant cell, callus, plant tissue or plant part, that contains all or part of at least one recombinant polynucleotide. In many cases, all or part of the recombinant polynucleotide is stably integrated into a chromosome or stable extra-chromosomal element, so that it is passed on to successive generations. For the purposes of the invention, a “recombinant polypeptide” is a polypeptide that has been altered by human intervention or produced or existing in an organism or in a location that is not its natural site. For example, a recombinant polypeptide is one that is produced or exists in a transgenic host cell. An example of a recombinant polypeptide is a polypeptide that is encoded by a recombinant polynucleotide. A recombinant polynucleotide is a polynucleotide that is substantially free of the nucleic acid sequences that normally flank the polynucleotide. For example, a cloned polynucleotide is considered a recombinant polynucleotide. Alternatively, a polynucleotide is considered recombinant if it has been altered by human intervention, or placed in a locus or location that is not its natural site, for example, a transgenic host.

Methods of Altering Plant Agronomic Traits

Methods of generating transgenic plants with altered agronomic traits are an aspect of the present invention. Plant agronomic traits are also and interchangeably referred to herein as developmental and phenotypic traits. Plant agronomic traits that may be altered according to the methods of the invention include one or more of the following traits: (1) time to reach flowering; (2) duration of flowering; (3) fruit yield; (4) seed yield; (5) root biomass; (6) seed size; (7) seed shape; (8) number of stem branches; and plant size. As used herein, the terms “altered,” “manipulated” and “modulated” are used interchangeably. When a plant agronomic trait is altered, this means that a transgenic plant produced by a method of the present invention has at least agronomic trait that is detectably different from a plant (e.g., a non-transgenic plant) that has not been produced by a method of the present invention (i.e., a plant that does not comprise an expression cassette of the present invention, as further defined herein).

An “altered” trait may be longer or shorted (if a temporal trait) than a non-altered trait; may be larger or smaller (if a physical size trait) than a non-altered trait; and may be more numerous or fewer (if a number trait) than a non-altered trait. By way of example, when the agronomic trait that is altered is duration of flowering, the duration of flowering in the altered plant may be longer or shorter than the duration of flowering in a non-altered plant. When the agronomic trait is root biomass, the root biomass of the altered plant may be larger or smaller than the root biomass, etc.

Specifically, the methods described herein relate to improving plant agronomic traits through the manipulation of the level of gene expression or protein activity of plant G-protein alpha and beta subunits. In particular, the invention is directed to the generation of plants with altered developmental and phenotypic traits through the manipulation of the level of gene expression or the activity of the gene products of plant endogenous G-protein alpha and beta genes that share sequence conservation with plant G-proteins AGB1 and GPA1.

The plant G-protein alpha and beta sequences useful in the present invention include those encoded by the Arabidopsis gene GPA1 and orthologs of GPA1, and the Arabidopsis gene AGB1 and orthologs of AGB1. The nucleotide sequence of the coding region of the Arabidopsis gene AGB1 is shown in SEQ ID NO:1 and the polypeptide sequence in SEQ ID NO:2 (GI557694). Similarly, the nucleotide sequence of the coding region of the Arabidopsis gene GPA1 is shown in SEQ ID NO:3 and the polypeptide sequence in SEQ ID NO:4 (GI15225278).

Numerous orthologs of the Arabidopsis gene AGB1 from multiple plant species were aligned according to the programs described above. These orthologs are listed below with the percent sequence identity and percent sequence similarity of the encoded proteins to AGB1 in parentheses: potato, Accession Nos. GI15778632 (81, 89.9), GI1771734 (81, 90.4), (SEQ ID NOs:5-8); tobacco, Accession Nos. GI10048265 (81, 90.4), GI1360092 (80, 89.9), GI1835163 (82, 90.4), GI1835161 (81, 90.7), (SEQ ID NOs:9-16); pea, Accession Nos. GI15733806 (80, 89.6), GI14929352 (78, 88.8), (SEQ ID NOs:17-20); wild-oat, Accession No. GI12935698 (73, 84.7), (SEQ ID NOs:21-22); rice, Accession No. GI1143525 (76, 86.6), (SEQ ID NOs:23-24); and maize, Accession No. GI1557696 (76, 86.3), (SEQ ID NOs:25-26).

Orthologs of the Arabidopsis gene GPA1 have also been described for multiple plant species. The orthologs were aligned similarly and are listed below with the percent sequence identity and percent sequence similarity of the encoded proteins to GPA1 in parentheses: potato, Accession Nos. GI18032046 (84, 92.7), GI18032048 (83, 91.3), GI1771736 (85, 93.4), (SEQ ID NOs:27-32); rice, Accession No. GI540533 (73, 85.9), GI862310 (73, 85.6), (SEQ ID NOs:33-36); tobacco, Accession Nos. GI18369802 (80, 89), GI18369798 (81,89.2), GI18369796 (83, 92.4), GI10048263 (84, 92.7), GI1749827 (77, 86.2), (SEQ ID NOs:37-46); pea, Accession Nos. GI2104773 (85, 93.2), GI2104771 (85, 92.9), (SEQ ID NOs:47-50); tomato, Accession No. GI71922 (84, 92.7), (SEQ ID NO:51); spinach, Accession No. GI3393003 (82, 90), (SEQ ID NOs:52-53); soybean, Accession No. GI1834453 (84, 93.5), GI439617 (82, 91.1), (SEQ ID NOs:54-57); yellow lupine, Accession No. GI1480298 (84, 92.7), (SEQ ID NOs:58-59); and Lotus japonicus, Accession No. GI499078 (86, 92.4), (SEQ ID NOs:60-61).

As indicated by the above data, plant gene orthologs of AGB1 and GPA1 share a very high degree of sequence identity and sequence conservation across a broad range of species. For example, the sequence identity and sequence similarity of the plant G protein subunits listed above ranges from 73-98% (sequence identity), 84.7-98.6% (sequence similarity) and 72-86% (sequence identity), 85.1-92.4% (sequence similarity), for Gβ and Gα respectively. Six different species are listed for AGB1 and nine different species are listed for GPA1.

Any nucleotide sequence encoding a plant ortholog of AGB1 or GPA1 or any sequence encoding a protein that is capable of altering the activity of an AGB1 or GPA1 ortholog is useful in the methods of the present invention. The nucleotide sequences of the present invention that encode plant orthologs of AGB1 and GPA1 include, but not limited to, the sequences listed above. Plant orthologs of AGB1 and GPA1 that are encompassed by the present invention are nucleotide sequences that encode polypeptide sequences that share at least 75%, 80%, 81%, 82%, 83%, 84%, 85%, 86%, 87%, 88%, 89%, 90%, 91%, 92%, 93%, 94%, 95%, 96%, 97%, 98%, up to 99% sequence similarity to AGB1 or GPA1.

For example, the nucleotide sequences for the AGB1 and GPA1 genes and the AGB1 and GPA1 orthologs listed above can be utilized to isolate homologous genes from other plants including, but not limited to, additional members of the genus Brassica, gymnosperms, sorghum, wheat, cotton, barley, sunflower, cucumber, alfalfa, etc., using methods well known in the art. In using techniques known in the art, all or part of the known coding sequence is used as a probe that selectively hybridizes to other coding sequences for orthologs of AGB1 and GPA1 that are present in a population of cloned genomic DNA fragments or cDNA fragments (i.e., genomic or cDNA libraries) from a chosen plant.

Techniques known in the art include hybridization screening of plated DNA libraries (either plaques or colonies) (Sambrook et al., eds. (1989) Molecular Cloning: A Laboratory Manual (2d ed., Cold Spring Harbor Laboratory Press, Plainview, N.Y.) and amplification by PCR using oligonucleotide primers corresponding to sequence domains conserved among the amino acid sequences (Innis et al. (1990) PCR Protocols, a Guide to Methods and Applications (Academic Press, New York). Generally, because leader peptides are not highly conserved between monocots and dicots, sequences can be utilized from the carboxy-terminal end of the protein as probes for the isolation of corresponding sequences from any plant. Nucleotide probes can be constructed and utilized in hybridization experiments as discussed above. In this manner, even gene sequences that are divergent in the amino-terminal region can be identified and isolated for use in the methods of the invention.

The manipulation of the level of gene expression or protein activity of plant G-protein alpha and beta subunits (e.g., AGB1 and GPA1 genes and AGB1 and GPA1 orthologs) of the present invention may be carried out by several techniques and methods that will be described in more detail herein. These techniques and methods include nucleotide insertion techniques that include but are not limited to antisense suppression, dsRNA suppression, insertion of inverted repeats, sense co-suppression, and sense over-expression. Suitable techniques and methods also include that include but are not limited to gene disruption techniques such as, for example, the use of ribozymes, site-directed and random (chemical or radiation-induced) mutagenesis, expression of a sense sequence containing a dominant site-directed mutation T-DNA or transposon insertions, and alteration of expression of target gene accessory proteins. Still other suitable techniques relate to the use of a tissue-preferred transactivation systems,.

In general, regardless of the particular technique or method used, the present methods for altering the level of gene expression or protein activity of plant G-protein alpha and beta subunits comprise introducing into a plant cell an expression cassette, where the expression cassette comprises: (1) a promoter that is operable within the plant cell; and (2) a nucleotide sequence for altering the level of gene expression or protein activity of plant G-protein alpha and beta subunits, wherein the nucleotide sequence is operably linked to the promoter.

Promoters useful in the expression cassettes of the invention include any promoter that is capable of initiating transcription in a plant cell. Such promoters include, but are not limited to, those that can be obtained from plants, plant viruses, and bacteria that contain genes that are expressed in plants, such as Agrobacterium and Rhizobium.

The promoter may be constitutive, inducible, developmental stage-preferred, cell type-preferred, tissue-preferred, organ-preferred, or a minimal promoter. Constitutive promoters are active under most conditions. Examples of constitutive promoters include the CaMV 19S and 35S promoters (Odell et al. (1985) Nature 313:810-812), the 2X CaMV 35S promoter (Kay et al. (1987) Science 236:1299-1302) the Sep1 promoter, the rice actin promoter (McElroy et al. (1990) Plant Cell 2:163-171), the Arabidopsis actin promoter, the ubiquitin promoter (Christensen et al. (1989) Plant Molec Biol 18:675-689); pEmu (Last et al. (1991) Theor Appl Genet 81:581-588), the figwort mosaic virus 35S promoter, the Smas promoter (Velten et al. (1984) EMBO J 3:2723-2730), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like. In a preferred embodiment of the invention, the promoter is the CaMV 35 S promoter.

The inducible promoters for use in the methods of the invention are active under certain environmental conditions, such as the presence or absence of a nutrient or metabolite, a chemical such as a steroid, heat or cold, light, pathogen attack, anaerobic conditions, and the like. For example, the hsp80 promoter from Brassica is induced by heat shock, the PPDK promoter is induced by light, the PR-1 promoter from tobacco, Arabidopsis, and maize are inducible by infection with a pathogen, and the Adh1 promoter is induced by hypoxia and cold stress.

Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to, fruit-preferred, ovule-preferred, male tissue-preferred, seed-preferred, integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters and the like.

Other male-preferred, tissue preferred, developmental stage preferred and/or inducible promoters include, but are not limited to, Ms45 (expressed in male tissue (U.S. Pat. No. 6,037,523)); Prha (expressed in root, seedling, lateral root, shoot apex, cotyledon, petiole, inflorescence stem, flower, stigma, anthers, and silique, and auxin-inducible in roots); VSP2 (expressed in flower buds, flowers, and leaves, and wound inducible); SUC2 (expressed in vascular tissue of cotyledons, leaves, and hypocotyl phloem, flower buds, sepals, and ovaries); AAP2 (silique-preferred); SUC1 (Anther and pistil preferred); AAP1 (seed preferred); Saur-AC1 (auxin inducible in cotyledons, hypocotyl and flower); Enod 40 (expressed in root, stipule, cotyledon, hypocotyl, and flower); amd VSP1 (expressed in young siliques, flowers and leaves).

Seed preferred promoters are preferentially expressed during seed development and/or germination. For example, seed preferred promoters can be embryo-preferred, endosperm preferred, and seed coat-preferred. (Thompson et al. (1989) BioEssays 10:108). Examples of seed preferred promoters include, but are not limited to, cellulose synthase (ceIA), Cim1, gamma-zein, globulin-1, maize 19 kD zein (cZ19B1), and the like.

Other promoters useful in the expression cassettes of the invention include, but are not limited to, the major chlorophyll a/b binding protein promoter, histone promoters, the prolifera promoter, the Ap3 promoter, the beta-conglycin promoter, the phaseolin promoter, the napin promoter, the soy bean lectin promoter, the maize 15 kD zein promoter, the 22 kD zein promoter, the 27 kD zein promoter, the gamma-zein promoter, the waxy, shrunken 1, shrunken 2 and bronze promoters, the Zm13 promoter (U.S. Pat. No. 5,086,169), the maize polygalacturonase promoters (PG) (U.S. Pat. Nos. 5,412,085 and 5,545,546) and the SGB6 promoter (U.S. Pat. No. 5,470,359), as well as synthetic or other natural promoters.

The invention discloses “tissue- and/or stage-preferred promoters, herein used interchangeably with “tissue- and/or developmental-preferred promoters,” that are useful for promoting plant RNA transcript expression at greater levels in the particular tissue, stage, or developmental point of the plant than in other tissues, stages, or developmental points of the plant. The tissue- and/or stage-preferred promoters of the invention are D2 (AAP2, X95623, SEQ ID NO:68); D3 (Suc1, AJ001364.1, SEQ ID NO:69); D4 (Suc2, X79702, SEQ ID NO:70); D5 (bZIP, X99747, SEQ ID NO:71); D6 (VSP2, AB006778, SEQ ID NO:72); D11 (GluB1, X54314, SEQ ID NO:73); and D19 (SLG13; S82574, SEQ ID NO:74).

Root-preferred promoters are well known to those of skill in the art. A particularly useful root-preferred promoter of the invention is the D5 bZIP promoter set forth in SEQ ID NO:71. Other useful root-preferred promoters of the invention are bZIP root-preferred promoters. The bZIP root-preferred promoters direct root-preferred expression of bZIP transcription factor proteins. The bZIP transcription factor proteins belong to the evolutionary class of basic domain/leucine zipper (bZIP) transcription factor proteins. Examples of bZIP root-preferred promoters are promoters that direct root-preferred expression in plants of orthologs of the Arabidopsis ATB2 gene (SEQ ID NO:75). The ATB2 gene is described in Rook et al. (1998) Plant Mol. Biol. 37:171-178, herein incorporated by reference in its entirety. An ortholog of the Arabidopsis ATB2 gene sequence set forth in SEQ ID NO:75 refers to a gene from a species of plant other than Arabidopsis whose gene product shares substantial sequence conservation to ATB2 and the ATB2 gene product set forth in SEQ ID NO:76. In one case, the bZIP root-preferred promoters of the invention direct root-preferred RNA transcript expression in a dicot plant. In another case the bZIP root-preferred promoters of the invention direct root-preferred RNA transcript expression in dicot and/or monocot plants. Other examples of useful root-preferred promoters of the invention include D2, D3, D4, D5, D6, D11, and D19.

The D5 bZIP promoter of the invention controls transcription of the Arabidopsis ATB2 open reading frame. The ATB2 genomic clone including the D5 promoter sequence was isolated by Rook et al. (1998) Plant Mol. Biol. 37:171-178, herein incorporated by reference in its entirety, using a procedure involving conserved sequence domains similar to that described above. In the methods of the invention, orthologs of the ATB2 gene are isolated using the procedure of Rook et al. for plants including, but not limited to, tomato, potato, pea, spinach, tobacco, soybean, sunflower, peanut, alfalfa, mint, cotton, rice, maize, oats, wheat, barley, sorghum, grasses, Brassica and Brassica napus. In this manner, the promoter sequences controlling the expression of the ATB2 orthologs are isolated. The promoter sequences controlling expression of ATB2 orthologs in plants are useful bZIP root-preferred promoters of the invention.

As described above, the manipulation of the level of gene expression or protein activity of plant G-protein alpha and beta subunits may be carried out by numerous techniques and methods. In one embodiment, nucleotide insertion techniques including but not limited to antisense suppression, dsRNA suppression, insertion of inverted repeats, sense co-suppression, and sense over-expression are used to manipulate the level of gene expression or protein activity of plant G-protein alpha and beta subunits, and thus provide plants with altered agronomic traits, where the traits are altered with respect to plants that that have not been genetically manipulated according to the methods described herein.

One particular embodiment of the invention is a method for altering a plant agronomic trait selected from the group consisting of time to flowering, duration of flowering in a plant, fruit yield, seed yield, root biomass, seed size, seed shape, number of stem branches, and size of a plant,. The method comprises introducing into a plant cell an expression cassette comprising a nucleotide sequence operably linked to a promoter that is operable within the plant cell, wherein the nucleotide sequence is selected from the group consisting of: (i) a nucleotide sequence antisense to a plant AGB1 or an AGB1 ortholog, (ii) a nucleotide sequence comprising an inverted repeat of AGB1 or an AGB1 ortholog, (iii) a nucleotide sequence encoding a dsRNA, the dsRNA comprising a first RNA complementary to at least 25 consecutive nucleotides of a plant AGB1 or an AGB1 ortholog and a second RNA substantially complementary to the first RNA, (iv) a nucleotide sequence that is AGB1 or an AGB1 ortholog, and (v) a nucleotide sequence that is GPA1 or a GPA1 ortholog. The method further comprises regenerating a plant that has a stably integrated expression cassette from the plant cell, wherein the regenerated plant has an altered agronomic trait.,

Use of antisense and sense nucleotide sequences for the silencing of plant genes is well known in the art. For antisense suppression of gene expression see particularly Inouye et al., U.S. Pat. Nos. 5,190,931 and 5,272,065; Albertsen et al., U.S. Pat. No. 5,478,369; Shewmaker et al., U.S. Pat. No. 5,453,566; Weintrab et al. (1985) Trends Gen. 1:22-25; and Bourque and Folk (1992) Plant Mol. Biol. 19:641-647. Antisense nucleotide sequences are particularly effective in manipulating metabolic pathways to alter the phenotype of an organism. Reduction in gene expression can be mediated at the DNA level and at transcriptional, post-transcriptional, or translational levels. For example, it is thought that dsRNA suppresses gene expression by both a post-transcriptional process and by DNA methylation. (Sharp & Zamore (2000) Science 287:2431-2433). Antisense polynucleotides, when introduced into a plant cell, are thought to specifically bind to their target polynucleotide and inhibit gene expression by interfering with transcription, splicing, transport, translation and/or stability. Antisense polynucleotides can be targeted to chromosomal DNA, to a primary RNA transcript or to a processed mRNA. Preferred target regions include splice sites and translation initiation and termination codons, and other sequences within the open reading frame.

It is understood that the antisense polynucleotides of the invention need not be completely complementary to the target gene or RNA (AGB1, GPA1 or an ortholog thereof), nor that they hybridize to each other along their entire length to modulate expression or to form specific hybrids. Furthermore, the antisense polynucleotides of the invention need not be full length with respect to the target gene or RNA. In general, greater homology can compensate for shorter polynucleotide length. Typically antisense molecules will comprise an RNA having 60-100% sequence identity with at least 8, 10, 12, 14, 15, 16, 17, 18, 19, 20, 25, 30, 35, 40, 45, 50, 75, 100, 200, 500, or at least 1000 consecutive nucleotides of the target gene. Preferably, the sequence identity will be at least 70%, more preferably at least 75%, 80%, 85%, 90%, 95%, 98% and most preferably at least 99%. Target genes include AGB1, GPA1 or an ortholog thereof, including the nucleotide sequences listed SEQ ID NOs:1-61.

Antisense polynucleotides may be designed to bind to exons, introns, exon-intron boundaries, the promoter and other control regions, such as the transcription and translational initiation sites. Methods for inhibiting plant gene expression using antisense RNA corresponding to entire and partial cDNA, 3′ non-coding regions, as well as relatively short fragments of coding regions are known in the art. (U.S. Pat. Nos. 5,107,065 and 5,254,800, the contents of which are incorporated by reference; Sheehy et al. (1988) Proc. Nat'l. Acad. Sci. USA 85:8805-8809; Cannon et al. (1990) Plant Mol. Biol. 15:39-47; and Chang et al. (1989) Proc. Nat'l. Acad. Sci. USA 86:10006-10010). Furthermore, Van der Krol et al. (1988) Biotechniques 6:958-976, describe the use of antisense RNA to inhibit plant genes in a tissue-specific manner.

Gene specific inhibition of expression in plants by an introduced sense polynucleotide is termed “co-suppression.” Methods for co-suppression are known in the art. Partial and full-length cDNAs have been used for the co-suppression of endogenous plant genes. (U.S. Pat. Nos. 4,801,340; 5,034,323; 5,231,020; and 5,283,184, the contents of each are herein incorporated by reference; Van der Kroll et al. (1990) The Plant Cell 2:291-299, Smith et al. (1990) Mol. Gen. Genetics 224:477-481; and Napoli et al. (1990) The Plant Cell 2:279-289).

For sense suppression, it is believed that introduction of a sense polynucleotide blocks transcription of the corresponding target gene. In the methods of the present invention, the sense polynucleotide will have at least 80%, 90%, 95% or more sequence identity with the target plant gene or RNA (AGB1, GPA1 or an ortholog thereof). The introduced sense polynucleotide need not be full length relative to the target gene or transcript. Preferably, the sense polynucleotide will have at least 95%, 96%, 97%, 98%, 99% or 100% sequence identity with at least 100 consecutive nucleotides of GPA1, AGB1 or an ortholog thereof, including the nucleotide sequences listed in SEQ ID NOs:1-61. The regions of identity comprise introns and and/or exons and untranslated regions. The introduced sense polynucleotide is stably integrated into a plant chromosome or extrachromosomal replicon.

In the case of the expression of sense polynucleotides in plants, the introduction of a sense polynucleotide may result in the up-regulation of the corresponding target gene. Thus, in another embodiment of the invention, the over-expression of sense polynucleotides corresponding to AGB1, GPA1 or an ortholog thereof, results in the up-regulation of the corresponding target gene. In this manner, the phenotype of a transgenic plant is altered through the increased expression of the target gene. In the methods of the invention, the sense polynucleotides will encode the amino acid sequence of the target plant protein or an amino acid sequence that is at least 90%, 95%, 98%, 99% or more identical to the target plant protein (GPA1, AGB1, or an ortholog thereof). Preferably, the sense polynucleotides (GPA1, AGB1 or orthologs thereof, including the polynucleotide sequences listed in SEQ ID NOs:1-61) will have 5 or fewer alterations in amino acid residues that are not highly conserved between species. The introduced sense polynucleotide is stably integrated into a plant chromosome or extrachromosomal replicon. In a preferred embodiment of the invention, the introduced sense polynucleotide encodes a GPA1 ortholog. An increased level of GPA1 in the cell promotes sequestration of the AGB1 subunit and mimics phenotypes observed in the agb1 mutants.

In another aspect, the invention provides a double-stranded RNA (dsRNA) for the post-transcriptional inhibition of a target plant gene. In the methods of the present invention, the dsRNA is specific for a target gene or RNA (AGB1, GPA1 or an ortholog thereof). Preferably, the dsRNA will be at least 25, 30, 35, 40, 45, 50, 55, 60, 65, 70, 75, 80, 85, 90, 95, or 100 base pairs in length (Hamilton & Baulcombe (1999) Science 286:950). Typically, the hybridizing RNAs of will be of identical length with no over hanging 5′ or 3′ ends and no gaps. However, dsRNAs having 5′ or 3′ overhangs of up to 100 nucleotides may be used in the methods of the present invention.

Thus, in one embodiment, the invention provides a dsRNA, comprising: a first ribonucleic acid having at least 95% complementary with at least 25, 30, 35, 40, 45, 50, 55, 60, 65, 70, 75, 80, 85, 90, 95, or 100 consecutive nucleotides (GPA1, AGB1 or an ortholog thereof including nucleotide sequences listed in SEQ ID NOs:1-61); and a second ribonucleic acid that is substantially complementary to the first ribonucleic acid.

The dsRNA may comprise ribonucleotides or ribonucleotide analogs, such as 2′-O-methyl ribosyl residues or combinations thereof. (U.S. Pat. Nos. 4,130,641 and 4,024,222). A dsRNA polyriboinosinic acid:polyribocytidylic acid is described in U.S. Pat. No. 4,283,393. Methods for making and using dsRNA are known in the art. One method comprises the simultaneous transcription of two complementary DNA strands, either in vivo, or in a single in vitro reaction mixture. (U.S. Pat. No. 5,795,715, the content of which is incorporated herein by reference). In the methods of the present invention, the dsRNA is expressed in a plant cell through the transcription of two complementary RNAs.

As set forth above, the manipulation of the level of gene expression or protein activity of plant G-protein alpha and beta subunits (e.g., AGB1 and GPA1 genes and AGB1 and GPA1 orthologs) of the present invention may also be carried out by causing a disruption in a gene in a plant cell. As defined above, the term “causing a disruption in a gene” is used herein to refer to a means of altering the expression of a gene. Suitable techniques and methods also include gene disruption techniques such as, for example, the use of ribozymes, site-directed and random (chemical or radiation-induced) mutagenesis, T-DNA or transposon insertions, and alteration of expression of target gene accessory proteins.

Thus, one embodiment of the invention is a method for altering a plant agronomic trait selected from the group consisting of time to flowering, duration of flowering in a plant, fruit yield, seed yield, root biomass, seed size, seed shape, number of stem branches, and size of a plant, the method comprising: a) causing a disruption in a gene in a plant cell other than Arabidopsis, wherein the gene is an AGB1 ortholog endogenous to the plant cell; and b) regenerating a plant from the plant cell, wherein the plant has a disruption in the endogenous gene and the plant exhibits an altered agronomic trait. Another embodiment relates to a method for altering a plant agronomic trait selected from the group consisting of time to flowering, duration of flowering in a plant, fruit yield, seed yield, root biomass, seed size, seed shape, number of stem branches, and size of a plant, the method comprising a) causing a disruption in a gene in a plant cell that is not Arabidopsis thaliana or Orzya sativa, wherein the gene is a GPA1 ortholog endogenous to the plant cell; and b)regenerating a plant from the plant cell, wherein the plant has a disruption in the endogenous gene and the plant exhibits an altered fruit and seed yield.

One such technology is the use of ribozymes. In the methods of the invention ribozymes are used to reduce the expression of a target gene or RNA that is AGB1, GPA1 or an ortholog thereof.

Methods for making and using ribozymes are known to those skilled in the art. (U.S. Pat. Nos. 6,025,167; 5,773,260; 5,695,992; 5,545,729; 4,987,071; and 5,496,698, the contents of which are incorporated herein by reference; Haseloff & Gerlach (1988) Nature 334:586-591; Van Tol et al. (1991) Virology 180:23; Hisamatsu et al. (1993) Nucleic Acids Symp. Ser. 29:173; Berzal-Herranz et al. (1993) EMBO J. 12:2567 (describing essential nucleotides in the hairpin ribozyme); Hampel & Tritz, (1989) Biochemistry 28:4929; Haseloff et al. (1988) Nature 334:585-591; Haseloff & Gerlach (1989) Gene 82:43 (describing sequences required for self-cleavage reactions); and Feldstein et al. (1989) Gene 82:53). For a review of various ribozyme motifs, and hairpin ribozyme in particular, see Ahsen & Schroeder (1993) Bioessays 15:299; Cech (1992) Curr. Opi. Struc. Bio. 2:605; and Hampel et al. (1993) Methods: A Companion to Methods in Enzymology 5:37.

The portion of the ribozyme that hybridizes to the target gene or RNA transcript (GPA1, AGB1 or an ortholog thereof) is typically at least 7 nucleotides in length. Preferably, this portion is at least 8, 9, 10, 12, 14, 16, 18 or 20 or more nucleotides in length. The portion of the ribozyme that hybridizes to the target need not be completely complementary to the target, as long as the hybridization is specific for the target. In a preferred embodiment, the ribozyme will contain a portion having at least 7 or 8 nucleotides that have 100% complementarity to a portion of the target RNA. In one embodiment, the target RNA transcript corresponds to AGB1, GPA1 or an ortholog thereof, including the nucleotide sequences listed in SEQ ID NOs:1-61.

Similarly, methods for the disruption of target plant genes (GPA1 or AGB1 orthologs or genes encoding proteins that regulate the activity of GPA1 or AGB1 orthologs) include T-DNA or transposon insertion methodologies. As part of the disease process, bacteria of the genus Agrobacterium transfer a segment of DNA to the nucleus of the host plant cell. This transferred DNA (T-DNA) integrates at random locations in the host genome. Transgenic plants with T-DNA integrations within the open reading frame or the promoter region of the target gene are identified using a polymerase chain reaction screening procedure that is well known by those skilled in the art. (Krysan et al. (1996) Proc. Nat'l. Acad. Sci. USA 93:8145-50).

Target gene inactivation is also accomplished via transposon insertion in the promoter or coding region of the gene. In the methods of the present invention, the transposon used to inactivate the gene is native to the species in which the mutagenesis is being conducted (e.g., Blauth et al. (2002) Plant Mol. Biol. 48:287-97) or derived from a heterologous species (e.g., Kohli et al. (2001) Mol. Genet. Genomics 266:1-11). In either case, a polymerase chain reaction method analogous to that described above is utilized to identify plant lines with the desired gene disruption. Insertional mutagenesis technologies are reviewed by Parinov & Sundaresan (2000) Curr. Opin. Biotechnol. 11:157-61; and Krysan, Young & Sussman (1999) Plant Cell 11:2283-90.

Other well-known gene disruption technologies for inhibition of a target plant gene can be used in the methods of the invention. One such method relates to directed or random mutagenesis of a target gene. Thus, in another embodiment of the invention, directed alteration of target GPA1 or AGB1 ortholog activity is performed through genetic manipulation of the cloned GPA1 or AGB1 ortholog cDNA coding region. The directed genetic manipulation of the cloned cDNA generates a mutation in a highly conserved region of the AGB1 or GPA1 ortholog target, resulting in a non-conservative amino acid substitution which inactivates or alters (i.e. increases or decreases) the activity of the target protein in a genetically dominant manner. Alternatively, directed genetic manipulation of cloned AGB1 or GPA1 ortholog cDNA is used to produce a deletion (so-called truncation), or addition of one or more amino acids to the amino-terminal and/or carboxy-terminal end of the AGB1 or GPA1 ortholog protein.

Methods for such directed genetic manipulations are generally known in the art. For example, amino acid sequence variants of the polypeptide can be prepared by mutations in the cloned DNA sequence encoding the native protein of interest. Methods for mutagenesis and nucleotide sequence alterations are well known in the art. (Walker & Gaastra, eds. (1983) Techniques in Molecular Biology (MacMillan Publishing Company, New York); Kunkel (1985) Proc. Natl. Acad. Sci. 82:488-492; Kunkel et al. (1987) Methods Enzymol. 154:367-382; Sambrook et al. (1989) Molecular Cloning: A Laboratory Manual (2d ed., Cold Spring Harbor Laboratory Press, Plainview, N.Y.; U.S. Pat. No. 4,873,192; and the references cited therein; all of which are herein incorporated by reference).

Specific examples for altering the activity of GPA1 orthologs through the transgenic expression of dominant site-directed mutations of GPA1 orthologs follow. Directed mutation of the conserved glutamine residue (corresponding to position 222 in GPA1) to a leucine in a GPA1 ortholog results in a GPA1 ortholog protein that is constitutively active. This mutation has been shown to reduce the rate of GTP hydrolysis by more than 100-fold, thereby maintaining the GTP-bound, active state of the protein (Masters et al. (1989) J. Biol. Chem. 264:15467-15474). Conversely, dominant negative mutations in Gα proteins that down-regulate the activity heterotrimeric G-proteins have also been identified. Substitution of the conserved glycine residue corresponding to GPA1 position 221 with alanine impairs binding of GDP. Substituting the conserved glutamic acid residue corresponding to GPA1 position 263 with alanine and substituting the conserved alanine residue corresponding to GPA1 position 355 with serine both reduce affinity for GTP and impair GTP-induced conformational change. GPA1 orthologs containing all three of these mutations in combination sequester Gβγ subunits and activated receptors, thereby blocking the signal transduction pathway in a dominant manner (liri et al. (1999) Proc. Natl. Acad. Sci. USA 96:499-504; Berlot (2002) J. Biol. Chem. 277: 21080-21085).

Thus, one embodiment of the invention includes methods for altering agronomic traits comprising introducing into a plant cell an expression cassette comprising a sense nucleotide sequence that is a GPA1 ortholog and that contains a dominant site-directed mutation; and regenerating a plant that has a stably integrated expression cassette from the plant cell, wherein the plant exhibits one or more of altered agronomic traits.

The methods of the invention include methods for disrupting a target gene (a GPA1 or AGB1 ortholog or genes encoding proteins that regulate the activity of GPA1 or AGB1 orthologs) in a plant using random mutagenesis. For the random mutagenesis of a target gene, the mutagenesis is performed using chemicals, irradiation, T-DNA, or transposon insertion. Thus, in another embodiment of the invention, mutagenesis of a GPA1 or AGB1 ortholog or genes encoding proteins that regulate the activity of GPA1 or AGB1 orthologs is performed randomly using either a chemical mutagen or through irradiation of the DNA. Inactivation of the target protein is accomplished by generating a mutation resulting in a non-conservative amino acid substitution in a highly conserved region of the target gene. Alternatively, target protein inactivation is obtained through alteration of any of the codons in the coding region of the target gene that result in the truncation of the protein. Plant lines containing mutations in AGB1 or GPA1 orthologs or genes encoding proteins that regulate the activity of GPA1 or AGB1 orthologs are identified by TILLING (McCallum et al. (2000) Nat. Biotechnol 18:455-457), or through phenotypic screening followed by molecular characterization of the inactive gene. Such techniques for the generation of random mutations in target genes are well known in the art. (Koncz, Chua & Schell, eds., (1993) Methods in Arabidopsis Research (World Scientific Publishing, River Edge, N.J.)).

In addition to the technologies mentioned above for altering target gene activity (GPA1, AGB1, or orthologs thereof), the invention also provides methods for modulating target gene activity via altered expression of accessory proteins in the plant cell. The accessory proteins of the invention belong to either of two diverse categories termed Activators of G-protein Signaling (AGS) and Regulators of G-protein Signaling (RGS).

AGS proteins are structurally diverse and are able to activate heterotrimeric G-proteins independently of a G-protein coupled receptor (reviewed by Cismowski et al. (2001) Life Sciences 68: 2301). As an example, AGS1 functions as a guanine nucleotide exchange factor, activating Gα by promoting the exchange of GDP for GTP. In contrast, AGS2 and AGS3 act independently of nucleotide exchange by Gα. AGS2 binds the Gβγ subunit and affects downstream signaling events by promoting and/or maintaining the dissociation of the Gα and Gβγ subunits. AGS3 functions as a guanine nucleotide dissociation inhibitor and stabilizes the GDP-bound form of Gα. The end result of AGS2 and AGS3 action is enhanced signaling activity of the free Gβγ subunit.

Greater than 20 genes belonging to the RGS family have been identified in mammals. Although the proteins encoded by these genes are structurally diverse, they share a conserved motif of ˜120 amino acids termed the RGS domain. The RGS domain interacts with activated G-proteins and accelerates GTP hydrolysis by as much as 2000 fold. Thus, RGS proteins modulate signaling activity by depleting the GTP-activated form of the Gα subunit, by changing signaling kinetics, or by changing signaling specificity (reviewed by Ross & Wilkie (2000) Ann. Rev. Biochem. 69:795).

Thus, one embodiment of the invention relates to a method for introducing into a plant cell an expression cassette comprising a nucleotide sequence that is antisense, sense, sense containing a dominant site-directed mutation, dsRNA, or an inverted repeat in relation to a plant nucleotide sequence that is an AGS1, AGS2, or AGS3 ortholog; or, alternatively, an expression cassette comprising a nucleotide sequence causing a disruption in a gene in a plant cell, wherein the gene is an AGS1, AGS2, or AGS3 ortholog endogenous to the plant cell. The method further comprises and regenerating a plant that has a stably integrated expression cassette or disrupted gene from the plant cell, wherein the plant exhibits an altered agronomic trait.

Another embodiment of the invention relates to a method for introducing into a plant cell an expression cassette comprising a nucleotide sequence that is antisense, sense, sense containing a dominant site-directed mutation, dsRNA, or an inverted repeat in relation to a plant nucleotide sequence that is an RGS ortholog; or, alternatively, an expression cassette comprising a nucleotide sequence causing a disruption in a gene in a plant cell, wherein the gene is an RGS ortholog endogenous to the plant cell. The method further comprises and regenerating a plant that has a stably integrated expression cassette or disrupted gene from the plant cell, wherein the plant exhibits an altered agronomic trait.

It is known in the art that additional flexibility in controlling heterologous gene expression in plants may be obtained by using DNA binding domains and response elements from heterologous sources (i.e., DNA binding domains from non-plant sources). Some examples of such heterologous DNA binding domains include the LexA and GAL4 DNA binding domains.

Tissue-preferred transactivation system in which the transgene to be expressed (target) is under the control of a minimal promoter linked to cis-acting upstream activator sequences (UAS) are known. Activation of the target transgene is provided by a synthetic transcription factor (driver) that specifically binds the UAS elements in the target gene promoter. Previous studies using this technology in plants have relied on constitutive or chemical-inducible promoters to control driver transgene expression. The utility of previously disclosed transactivation systems is expanded as described herein by developing a collection of transgenic driver lines that can be used to control tissue- and developmental-stage-preferred expression of target transgenes containing Gal4-UAS elements.

In light of this knowledge, still other methods of manipulating of the level of gene expression or protein activity of plant G-proteins relates to the use of a tissue-preferred transactivating system. The methods are directed to the generation of transgenic plants with improved agronomical traits as a result of altering the expression level of a specific endogenous gene in a tissue-preferred manner. In one aspect, these methods are directed to the generation of transgenic plants with improved agronomical traits by reducing the level of gene expression in root tissue of plant endogenous G-protein beta genes. In particular embodiment, the G-protein beta genes share sequence conservation with the Arabidopsis AGB1 gene. These methods find particular use in the generation of transgenic plants having increased root biomass.

A particular embodiment is a method of generating a transgenic plant having increased root biomass, the plant comprising a driver cassette comprising a synthetic chimeric transcription factor open reading frame operably linked to a root-preferred promoter, and a target cassette comprising a nucleotide operably linked to a minimal promoter operably linked to at least one cognate upstream activating sequence, wherein the nucleotide sequence is selected from the group consisting of (i) at least a portion of an AGB1 gene sequence set forth in SEQ ID NO:1 in the antisense orientation and (ii) an ortholog of an AGB1 gene sequence set forth in SEQ ID NO:1 in the antisense orientation. In these methods, each of the driver and the target cassettes is stably integrated in the genome of the plant, and the plant has an increased root biomass.

As the methods of the invention are directed to reducing the level of gene expression of plant endogenous G-protein beta genes in root tissue, orthologs of the Arabidopsis AGB1 gene (SEQ ID NO:1) and root-preferred promoters are of particular use in the methods of the invention. Thus, any nucleotide sequence encoding a plant ortholog of the AGB1 gene is useful in the methods of the present invention. An ortholog of the AGB1 gene sequence set forth in SEQ ID NO:1 refers to a gene from a species of plant other than Arabidopsis that shares substantial sequence conservation to AGB1 and the AGB1 gene product set forth in SEQ ID NO:2.

In one embodiment, the synthetic chimeric transcription factor open reading frame is, for example, a GAL4/VP16 open reading frame. In this embodiment, the minimal promoter is preferably operably linked to an upstream activation site comprising four DNA-binding domains of the yeast transcriptional activator GAL4. (Schwechheimer et al. (1998) Plant Mol. Biol. 36:195-204).

Any of the numerous root-preferred promoters as set forth above may be used in this particular method. In one embodiment, the root-preferred promoter is a bZIP root-preferred promoter, as defined herein. In another embodiment, the root-preferred promoter is a D5 bZIP promoter, as defined herein.

Thus, one particular embodiment of the invention is directed to a method for producing a transgenic plant having increased root biomass comprising generating a transgenic plant comprising a driver cassette comprising a GAL4/VP16 open reading frame operably linked to a bZIP root-preferred promoter, and a target cassette comprising at least a portion of an AGB1 gene sequence set forth in SEQ ID NO:1 in the antisense orientation operably linked to a minimal promoter operably linked to at least one GAL4 upstream activating sequence, wherein each of the driver and the target cassettes is stably integrated in the genome of the plant and the plant has an increased root biomass. In a related embodiment of the invention, the target cassette comprises at least a portion of an ortholog of an AGB1 gene sequence set forth in SEQ ID NO:1.

Another specific embodiment of the invention is directed to a transgenic plant having increased root biomass, the plant comprising, stably integrated in its genome, a driver cassette comprising a synthetic chimeric transcription factor open reading frame operably linked to a D5 bZIP promoter; and a target cassette comprising at least a portion of an AGB1 gene sequence set forth in SEQ ID NO:1 in the antisense orientation operably linked to a minimal promoter operably linked to at least one cognate upstream activating sequence. In a related embodiment of the invention, the target cassette comprises at least a portion of an ortholog of an AGB1 gene sequence set forth in SEQ ID NO:1.

The methods of the present invention are useful for altering agronomic traits in a broad variety of plant species, and are thus useful in generating a broad variety of transgenic plant species. One skilled in the art will be able to select which plant species to utilize in conjunction with the present invention based upon the agronomic traits that the artisan wishes to alter in accordance with the invention.

In general, all methods of the invention are useful in dicots, monocots, and plants that are members of the genus Brassica, such as Brassica napus.

For flowering traits such as time to flower or duration of flowering, methods of the invention are particularly useful for ornamental flowering plants and field crops such as maize, oats, soybean, wheat, barley, canola, and other commercially important field crops. For agronomic traits such as fruit yield, seed yield, root biomass, and/or seed size in plants, the methods of the invention are particularly useful for increasing fruit yield and/or decreasing seed size in plants that produce fruit such as apples, oranges, grapes, strawberries, blueberries, and other fruit-bearing plants. The methods of the invention are particularly useful for increasing seed yield and/or seed size in cereal crops such as rice, maize, oats, soybean, wheat, barley etc, and in the crop Brassica napus to increase the yield of canola oil. Methods of the present invention that increase yields in fruit, grain, or oil is possible without a corresponding increase in plant material and the potential increase in crop care and management.

For agronomic traits such as seed shape, methods of the invention are particularly useful for cereal crops such as rice, maize, oats, soybean, wheat, barley, and other commercially important cereal crops.

For agronomic traits such as number of stem branches and/or altering the size of plants, the methods of the invention are useful in tree and gymnosperm species in addition to other plants such as dicots, monocots, plants that are members of the genus Brassica. The methods of the invention are particularly useful in timber trees for which reduced branching is desirable, trees such as gymnosperms, pines, and hardwood trees. The methods of the invention are also useful in ornamental plants, such as fruit trees, for which reduced size and/or reduced branching is desirable.

For agronomic traits such as root biomass, methods of the invention are particularly useful monocots, dicots, vegetable crops, tomato, potato, pea, spinach, tobacco, soybean, sunflower, peanut, alfalfa, mint, cotton, rice, maize, oats, wheat, barley, sorghum, grasses, Brassica, Brassica napus, and Arabidopsis.

Transgenic plants having altered agronomic traits are thus an aspect of the present invention. The present invention encompasses transgenic plants having stably integrated into their genome an expression cassette comprising a nucleotide sequence that is antisense, sense, dsRNA, a ribozyme, or an inverted repeat to a plant nucleotide sequence that is AGB1 or an AGB1 ortholog. Further encompassed by the present invention are transgenic plants having a disruption in a gene that is an AGB1 ortholog endogenous to the plant. The transgenic plants of the invention include dicots, monocots, plants that are members of the genus Brassica, particularly Brassica napus, trees, and gymnosperms.

Also included in the present invention are transgenic plants having stably integrated into their genome an expression cassette comprising a nucleotide sequence that is antisense, sense, sense containing a dominant site-directed mutation, dsRNA, a ribozyme, or an inverted repeat to a nucleotide sequence that is GPA1 or a GPA1 ortholog. In addition, the invention includes transgenic plants having a disruption in a gene that is a GPA1 ortholog endogenous to the plant. The invention is particularly directed to transgenic plants, and seed thereof, that are monocots, dicots, or a member of the genus Brassica, particularly Brassica napus.

Other transgenic plants encompassed by the present invention include transgenic plants having stably integrated into their genome an expression cassette comprising a sense nucleotide sequence that is a GPA1 ortholog and that contains a dominant site-directed mutation.

Transgenic plants having stably integrated into their genome an expression cassette comprising a nucleotide sequence that is antisense, sense, sense containing a dominant site-directed mutation, dsRNA, a ribozyme, or an inverted repeat to a nucleotide sequence that is an AGS1, AGS2, or AGS3 ortholog are an aspect of the invention. Further included are transgenic plants that have a disruption in a gene that is an AGS1, AGS2, or AGS3 ortholog endogenous to the plant.

Transgenic plants having stably integrated into their genome an expression cassette comprising a nucleotide sequence that is antisense, sense, sense containing a dominant site-directed mutation, dsRNA, a ribozyme, or an inverted repeat to a nucleotide sequence that is an RGS ortholog are an aspect of the invention. Further included are transgenic plants that have a disruption in a gene that is an RGS ortholog endogenous to the plant.

Transgenic plants of the invention that have increased root biomass may comprise a separate driver cassette, for root-preferred expression of a synthetic chimeric transcription factor, and a target cassette for the transcription factor promoted antisense expression of an AGB1 gene sequence, or ortholog thereof. The transgenic plants of the invention are monocots, dicots, vegetable crops, tomato, potato, pea, spinach, tobacco, soybean, sunflower, peanut, alfalfa, mint, cotton, rice, maize, oats, wheat, barley, sorghum, grasses, Brassica, Brassica napus, and Arabidopsis.

Thus, the present invention encompasses transgenic plants having increased root biomass, the plants comprising, stably integrated in their genome, a driver cassette comprising an synthetic chimeric transcription factor open reading frame (e.g., a GAL4/VP16 open reading frame) operably linked to a root-preferred promoter (e.g., a bZIP or D5 bZIP promoter); as well as a target cassette comprising at least a portion of an AGB1 gene sequence set forth in SEQ ID NO:1 in the antisense orientation operably linked to a minimal promoter operably linked to at least one cognate upstream activating sequence (e.g., GAL4 upstream activating sequence). In a related embodiment of the invention, the target cassette comprises at least a portion of an ortholog of an AGB1 gene sequence set forth in SEQ ID NO:1.

Another embodiment of the invention provides a transgenic plant having increased root biomass, the plant comprising, stably integrated in its genome, a driver cassette comprising a GAL4/VP16 open reading frame operably linked to a bZIP root-preferred promoter; and a target cassette comprising at least a portion of an AGB1 gene sequence set forth in SEQ ID NO:1 in the antisense orientation operably linked to a minimal promoter operably linked to at least one GAL4 upstream activating sequence. In a related embodiment of the invention, the target cassette comprises at least a portion of an ortholog of an AGB1 gene sequence set forth in SEQ ID NO:1.

Still another embodiment of the invention provides a transgenic plant having increased root biomass, the plant comprising, stably integrated in its genome, a driver cassette comprising a GAL4/VP16 open reading frame operably linked to a root-preferred promoter; and a target cassette comprising at least a portion of an AGB1 gene sequence set forth in SEQ ID NO:1 in the antisense orientation operably linked to a minimal promoter operably linked to at least one GAL4 upstream activating sequence. In a related embodiment of the invention, the target cassette comprises at least a portion of an ortholog of an AGB1 gene sequence set forth in SEQ ID NO:1.

Transgenic plants of the present invention are made according to methods set forth herein and other methods known in the art.

The polynucleotides of the invention may be introduced into any plant or plant cell. By plants is meant angiosperms (monocotyledons and dicotyledons) and gymnosperms, and the cells, organs and tissues thereof. Methods for the introduction of polynucleotides into plants and for generating transgenic plants are known to those skilled in the art. (Weissbach & Weissbach (1988) Methods for Plant Molecular Biology, Academic Press, N.Y.; Grierson & Corey (1988) Plant Molecular Biology, 2d., Blackie, London; Miki et al. (1993) Procedures for Introducing Foreign DNA into Plants, CRC Press, Inc. pp.67-80).

Vectors containing the expression cassettes of the invention are used in the methods of the invention. By “vector” it is intended to mean a polynucleotide sequence that is able to replicate in a host cell. Preferably, the vector contains genes that serve as markers useful in the identification and/or selection of transformed cells. Such markers include, but are not limited to, barnase (bar), G418, hygromycin, kanamycin, bleomycin, gentamicin, and the like. The vector can comprise DNA or RNA and can be single or double stranded, and linear or circular. Various plant expression vectors and reporter genes are described in Gruber et al. in Methods in Plant Molecular Biology and Biotechnology, Glick et al., eds, CRC Press, pp.89-119, 1993; and Rogers et al. (1987) Meth Enzymol 153:253-277. In a preferred embodiment, the vector is an E. coli/A. tumefaciens binary vector. In another preferred embodiment of the invention the expression cassette is inserted between the right and left T-DNA borders of an Agrobacterium Ti plasmid.

The expression cassettes of the invention may be covalently liked to a polynucleotide encoding a selectable or screenable marker. Examples of such markers include genes encoding drug or herbicide resistance, such as hygromycin resistance (hygromycin phosphotransferase (HPT)), spectinomycin (encoded by the aada gene), kanamycin and gentamycin resistance (neomycin phosphotransferase (nptII)), streptomycin resistance (streptomycin phosphotransferase gene (SPT)), phosphinothricin or basta resistance (barnase (bar)), chlorsulfuron reistance (acetolactase synthase (ALS)), chloramphenicol resistance (chloramphenicol acetyl transferase (CAT)), G418 resistance, lincomycin resistance, methotrexate resistance, glyphosate resistance, and the like. In addition, the expression cassettes of the invention may be covalently linked to genes encoding enzymes that are easily assayed, for example, luciferase, alkaline phosphatase, beta-galactosidase (beta-gal), beta-glucuronidase (GUS), and the like.

Methods include, but are not limited to, electroporation (Fromm et al. (1985) Proc Natl Acad Sci 82:5824; Riggs et al. (1986) Proc. Nat'l. Acad. Sci. USA 83:5602-5606); particle bombardment (U.S. Pat. Nos. 4,945,050 and 5,204,253, the contents of which are herein incorporated by reference; Klein et al. (1987) Nature 327:70-73; McCabe et al. (1988) Biotechnology 6:923-926); microinjection (Crossway (1985) Mol Gen. Genet. 202:179-185; Crossway et al. (1986) Biotechniques 4:320-334); silicon carbide-mediated DNA uptake (Kaeppler et al. (1990) Plant Cell Reporter 9:415-418); direct gene transfer (Paszkowski et al. EMBO J. 3:2717-2722); protoplast fusion (Fraley et al. (1982) Proc. Nat'l. Acad. Sci. USA 79:1859-1863); polyethylene glycol precipitation (Paszowski et al.(1984) EMBO J. 3:2717-2722; Krens et al (1982) Nature 296:72-74); silicon fiber delivery; agroinfection (U.S. Pat. No. 5,188,958, incorporated herein by reference; Freeman et al. (1984) Plant Cell Physiol. 25:1353 (liposome-mediated DNA uptake); Hinchee et al. (1988) Biotechnology 6:915-921; Horsch et al. (1984) Science 233:496-498; Fraley et al. (1983) Proc. Nat'l. Acad. Sci. USA 80:4803; Hernalsteen et al. (1984) EMBO J. 3:3039-3041; Hooykass-Van Sloteren et al. (1984) Nature 311:763-764; Grimsley et al. (1987) Nature 325:1677-1679; Gould et al. (1991) Plant Physiol. 95:426-434; Kindle (1990) Proc. Nat'l. Acad. Sci. USA 87:1228 (vortexing method); Bechtold et al. (1995) In Gene Transfer to Plants, Potrykus et al., eds., Springer-Verlag, NewYork, N.Y. pp19-23 (vacuum infiltration); Schell (1987) Science 237:1176-1183; and Plant Molecular Biology Manual, Gelvin & Schilperoort, eds., Kluwer, Dordrecht, 1994).

Preferably, the polynucleotides of the invention are introduced into a plant cell by agroinfection. In this method, a DNA construct comprising a polynucleotide of the invention is inserted between the right and left T-DNA borders in an Agrobacterium tumefaciens vector. The virulence proteins of the A. tumefaciens host cell will mediate the transfer of the inserted DNA into a plant cell infected with the bacterium. As an alternative to the A. tumefaciens/Ti plasmid system, Agrobacterium rhizogenes-mediated transformation may be used. (Lichtenstein & Fuller in: Genetic Engineering, Volume 6, Ribgy, ed., Academic Press, London, 1987; Lichtenstein & Draper, in DNA Cloning, Volume 2, Glover, ed., IRI Press, Oxford, 1985).

If one or more plant gametes are transformed, transgenic seeds and plants can be produced directly. For example, a method of producing transgenic seeds and plants involves agroinfection of the flowers and collection of the transgenic seeds produced from the agroinfected flowers. Alternatively, transformed plant cells can be regenerated into plants by methods known to those skilled in the art. (Evans et al, Handbook of Plant Cell Cultures, Vol I, MacMollan Publishing Co. New York, 1983; and Vasil, Cell Culture and Somatic Cell Genetics of Plants, Acad. Press, Orlando, Vol 11, 1986).

Once a transgenic plant has been obtained, it may be used as a parent to produce progeny plants and plant lines. Conventional plant breeding methods can be used, including, but not limited to, crossing and backcrossing, self-pollination, and vegetative propagation. Techniques for breeding plants are known to those skilled in the art. The progeny of a transgenic plant are included within the scope of the invention, provided that the progeny contain all or part of the transgenic construct. Progeny may be generated by both asexual and sexual methods. Progeny of a plant include transgenic seeds, subsequent generations of the transgenic plant, and the seeds thereof.

Thus, one embodiment of the invention comprises using conventional breeding methods and/or successive iterations of genetic transformation to produce plant lines with genotypes including, but not limited to: simultaneous mutation or disruption of both AGB1 and GPA1 (or othologs thereof), simultaneous over-expression of AGB1 and GPA1 (or othologs thereof), over-expression of AGB1 (or an ortholog thereof) in a gpa1 or gpa1 ortholog mutant background, and over-expression of GPA1 (or an ortholog thereof) in an agb1 or agb1 ortholog mutant background; and phenotypes including one or more of: altered time to reach and duration of flowering, altered fruit yield, altered seed yield, altered root biomass, altered seed size and shape, altered number of stem branches, and altered plant size.

The transgenic plants of the invention are monocots or dicots, and are preferably dicots. The transgenic plants are preferably vegetable crops, tomato, potato, pea, spinach, tobacco, soybean, sunflower, peanut, alfalfa, mint, cotton, rice, maize, oats, wheat, barley, sorghum, grasses, Brassica, Brassica napus, and Arabidopsis, although transgenic plants may be of numerous species as set forth above.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Phenomics Profiling of GPA1 and AGB1 Mutants Throughout Development

### Generation of Mutant gpa1 and agb1 Transgenic Lines

Mutant alleles of the Arabidopsis GPA1 and AGB1 genes have been derived from independent T-DNA insertions near the middle of the genes (Ullah et al. (2001) Science 292: 2066-2069). The GPA1 alleles, gpa1-1 and gpa1-2, are in the Ws genetic background. Neither of the alleles is able to accumulate GPA1 protein to detectable levels. The AGB1 alleles, agb1-1 and agb1-2, are in the CoI-0 genetic background. agb1-1 is the result of a point mutation that prevents splicing of the first intron of the gene (Lease et al. (2001) Plant Cell 13: 2631-2641). This allele accumulates unspliced AGB1 transcript, but may make a truncated protein product. agb1-2 is the result of a T-DNA insertion in the fourth exon of the gene. This mutant fails to accumulate an AGB1 transcript.

### Phenotypic Profiling

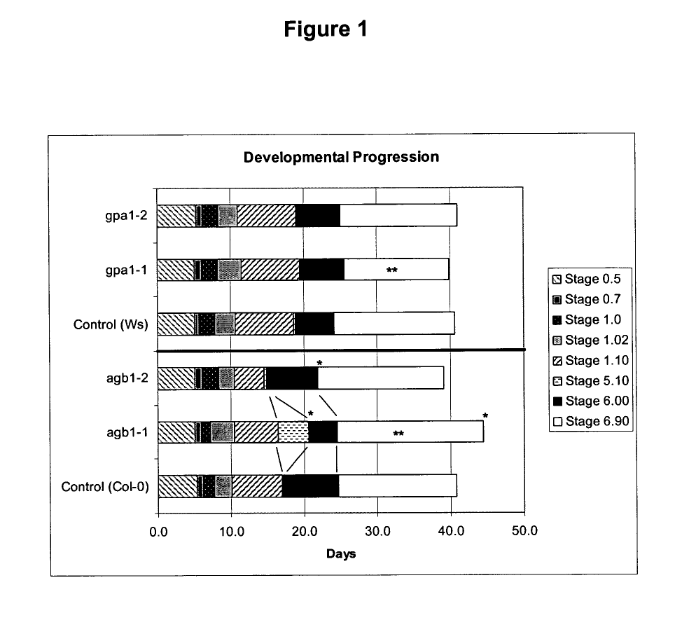

All four mutant lines, grown side by side with their corresponding wild type ecotypes, were subjected to an exhaustive phenotype profiling from seedling to senescence using the Paradigm Genetics, Inc. phenotypic analysis platform (Boyes et al. (2001) The Plant Cell 13:1499-1510, incorporated herein by reference). A set of 38 quantitative measurements were made at defined growth stages during Arabidopsis development and mean values of these traits in the mutants were tested for significant deviation from corresponding values of the wild type by pairwise two sample T-test. Mean values were derived from the analysis of 14 replicate plants per trait on average (details provided in Tables 1 & 2 and FIG. 1). The T-test results indicate the normalized difference between the mean response for the mutant and the mean response for the wild type and can be represented in units of standard error. A value of zero indicates concordance with the wild type trait value, while positive and negative T values indicate the relative degree to which the mutant trait value is larger or smaller, respectively. In this data set, T values greater than 2 standard errors from the wild-type mean are expected to occur by chance less than 5% of the time (p<0.05).

TABLE 1
Data from Early Plant Analysis and Phenomics Screens

| Trait | Line | Units | Mean | Std Dev | T test | n | Df | P valu |
|---|---|---|---|---|---|---|---|---|
| Days to Can flower buds be seen? | Col | Days | 19.5 | 2.7 | n.a. | 43 | n.a. | n.a. |
| Days to Can flower buds be seen? | agb1-1 | Days | 24.1 | 1.3 | 7.7 | 23 | 64 | 0.0000 |
| Days to Can flower buds be seen? | agb1-2 | Days | 18.5 | 1.2 | −1.8 | 25 | 66 | 0.0815 |
| Days to Can flower buds be seen? | WS | Days | 22.2 | 1.1 | n.a. | 38 | n.a. | n.a. |
| Days to Can flower buds be seen? | gpa1-1 | Days | 22.0 | 0.0 | −0.7 | 14 | 51 | 0.4873 |
| Days to Can flower buds be seen? | gpa1-2 | Days | 22.0 | 0.0 | −0.7 | 13 | 50 | 0.5035 |
| Days to Has flower production stopped? | Col | Days | 42.9 | 1.6 | n.a. | 13 | n.a. | n.a. |
| Days to Has flower production stopped? | agb1-1 | Days | 48.0 | 0.0 | 9.7 | 9 | 21 | 0.0000 |
| Days to Has flower production stopped? | agb1-2 | Days | 42.7 | 1.7 | −0.4 | 9 | 20 | 0.7200 |
| Days to Has flower production stopped? | WS | Days | 44.2 | 2.8 | n.a. | 11 | n.a. | n.a. |
| Days to Has flower production stopped? | gpa1-1 | Days | 42.3 | 1.5 | −1.5 | 6 | 15 | 0.1512 |
| Days to Has flower production stopped? | gpa1-2 | Days | 44.4 | 2.3 | 0.2 | 10 | 19 | 0.8459 |
| Days to Is first flower open? | Col | Days | 27.2 | 1.8 | n.a. | 39 | n.a. | n.a. |
| Days to Is first flower open? | agb1-1 | Days | 28.1 | 1.1 | 2.1 | 23 | 60 | 0.0402 |
| Days to Is first flower open? | agb1-2 | Days | 25.4 | 1.1 | −4.6 | 25 | 62 | 0.0000 |
| Days to Is first flower open? | WS | Days | 27.6 | 2.0 | n.a. | 29 | n.a. | n.a. |
| Days to Is first flower open? | gpa1-1 | Days | 28.0 | 0.0 | 0.9 | 20 | 48 | 0.3674 |
| Days to Is first flower open? | gpa1-2 | Days | 28.0 | 0.0 | 0.9 | 19 | 47 | 0.3798 |
| Distance across open flower | Col | mm | 4.1 | 0.4 | n.a. | 13 | n.a. | n.a. |
| Distance across open flower | agb1-1 | mm | 3.1 | 0.1 | −8.9 | 9 | 20 | 0.0000 |
| Distance across open flower | agb1-2 | mm | 3.2 | 0.1 | −8.2 | 10 | 21 | 0.0000 |
| Distance across open flower | WS | mm | 3.4 | 0.3 | n.a. | 14 | n.a. | n.a. |
| Distance across open flower | gpa1-1 | mm | 3.5 | 0.2 | 0.1 | 8 | 20 | 0.9241 |
| Distance across open flower | gpa1-2 | mm | 3.6 | 0.6 | 0.8 | 10 | 22 | 0.4220 |
| Dry weight of rosette (stage 6.9) | Col | g | 0.1635 | 0.0390 | n.a. | 14 | n.a. | n.a. |
| Dry weight of rosette (stage 6.9) | agb1-1 | g | 0.1750 | 0.0263 | 0.8 | 9 | 21 | 0.4452 |
| Dry weight of rosette (stage 6.9) | agb1-2 | g | 0.1222 | 0.0189 | −3.1 | 10 | 22 | 0.0054 |
| Dry weight of rosette (stage 6.9) | WS | g | 0.1131 | 0.0263 | n.a. | 14 | n.a. | n.a. |
| Dry weight of rosette (stage 6.9) | gpa1-1 | g | 0.1408 | 0.0298 | 2.3 | 8 | 20 | 0.0347 |
| Dry weight of rosette (stage 6.9) | gpa1-2 | g | 0.1571 | 0.0368 | 3.4 | 10 | 22 | 0.0024 |
| Dry weight of siliques (stage 6.9) | Col | g | 0.3036 | 0.0664 | n.a. | 15 | n.a. | n.a. |
| Dry weight of siliques (stage 6.9) | agb1-1 | g | 0.4856 | 0.1077 | 5.2 | 9 | 22 | 0.0000 |
| Dry weight of siliques (stage 6.9) | agb1-2 | g | 0.3689 | 0.0550 | 2.6 | 10 | 23 | 0.0170 |
| Dry weight of siliques (stage 6.9) | WS | g | 0.4308 | 0.1057 | n.a. | 14 | n.a. | n.a. |
| Dry weight of siliques (stage 6.9) | gpa1-1 | g | 0.4730 | 0.0610 | 1.0 | 8 | 20 | 0.3153 |
| Dry weight of siliques (stage 6.9) | gpa1-2 | g | 0.5981 | 0.1317 | 3.5 | 10 | 22 | 0.0023 |
| Dry weight of stem (stage 6.9) | Col | g | 0.2613 | 0.0528 | n.a. | 15 | n.a. | n.a. |
| Dry weight of stem (stage 6.9) | agb1-1 | g | 0.3180 | 0.0676 | 2.2 | 8 | 21 | 0.0368 |
| Dry weight of stem (stage 6.9) | agb1-2 | g | 0.2007 | 0.0317 | −3.2 | 10 | 23 | 0.0036 |
| Dry weight of stem (stage 6.9) | WS | g | 0.3978 | 0.0388 | n.a. | 14 | n.a. | n.a. |
| Dry weight of stem (stage 6.9) | gpa1-1 | g | 0.3810 | 0.0804 | −0.7 | 8 | 20 | 0.5128 |
| Dry weight of stem (stage 6.9) | gpa1-2 | g | 0.4177 | 0.0665 | 0.9 | 10 | 22 | 0.3644 |
| Lateral roots per seedling (d12) | Col | count | 7.3 | 2.4 | n.a. | 28 | n.a. | n.a. |
| Lateral roots per seedling (d12) | agb1-1 | count | 7.5 | 1.8 | 0.6 | 39 | 65 | 0.5773 |
| Lateral roots per seedling (d12) | agb1-2 | count | 10.5 | 2.9 | 4.7 | 31 | 57 | 0.0000 |
| Lateral roots per seedling (d12) | WS | count | 8.8 | 2.0 | n.a. | 40 | n.a. | n.a. |
| Lateral roots per seedling (d12) | gpa1-1 | count | 9.8 | 3.0 | 1.6 | 32 | 70 | 0.1132 |
| Lateral roots per seedling (d12) | gpa1-2 | count | 8.4 | 1.9 | −0.8 | 35 | 73 | 0.4024 |
| Length of peduncle of 2nd flower | Col | mm | 12.8 | 1.4 | n.a. | 15 | n.a. | n.a. |
| Length of peduncle of 2nd flower | agb1-1 | mm | 14.6 | 1.6 | 2.9 | 9 | 22 | 0.0075 |
| Length of peduncle of 2nd flower | agb1-2 | mm | 13.0 | 1.3 | 0.4 | 10 | 23 | 0.6915 |
| Length of peduncle of 2nd flower | WS | mm | 16.5 | 3.0 | n.a. | 14 | n.a. | n.a. |
| Length of peduncle of 2nd flower | gpa1-1 | mm | 32.8 | 3.4 | 11.8 | 8 | 20 | 0.0000 |
| Length of peduncle of 2nd flower | gpa1-2 | mm | 34.4 | 4.5 | 11.9 | 10 | 22 | 0.0000 |
| Length of primary root (d10) | Col | mm | 19.3 | 3.4 | n.a. | 33 | n.a. | n.a. |
| Length of primary root (d10) | agb1-1 | mm | 18.8 | 2.5 | −0.7 | 39 | 70 | 0.5029 |
| Length of primary root (d10) | agb1-2 | mm | 25.1 | 4.7 | 5.7 | 32 | 63 | 0.0000 |
| Length of primary root (d10) | WS | mm | 20.0 | 3.0 | n.a. | 40 | n.a. | n.a. |
| Length of primary root (d10) | gpa1-1 | mm | 21.1 | 4.6 | 1.3 | 40 | 78 | 0.1898 |
| Length of primary root (d10) | gpa1-2 | mm | 20.2 | 3.9 | 0.3 | 36 | 74 | 0.7598 |
| Length of primary root (d12) | Col | mm | 40.4 | 6.0 | n.a. | 33 | n.a. | n.a. |
| Length of primary root (d12) | agb1-1 | mm | 36.4 | 4.2 | −3.3 | 39 | 70 | 0.0015 |
| Length of primary root (d12) | agb1-2 | mm | 48.0 | 6.9 | 4.8 | 31 | 62 | 0.0000 |
| Length of primary root (d12) | WS | mm | 42.7 | 5.2 | n.a. | 40 | n.a. | n.a. |
| Length of primary root (d12) | gpa1-1 | mm | 40.9 | 5.6 | −1.4 | 38 | 76 | 0.1666 |
| Length of primary root (d12) | gpa1-2 | mm | 41.4 | 5.8 | −1.0 | 35 | 73 | 0.3164 |
| Length of primary root (d14) | Col | mm | 58.2 | 8.6 | n.a. | 33 | n.a. | n.a. |
| Length of primary root (d14) | agb1-1 | mm | 54.1 | 4.7 | −2.5 | 38 | 69 | 0.0135 |
| Length of primary root (d14) | agb1-2 | mm | 63.3 | 11.6 | 2.0 | 31 | 62 | 0.0510 |
| Length of primary root (d14) | WS | mm | 66.0 | 5.3 | n.a. | 40 | n.a. | n.a. |
| Length of primary root (d14) | gpa1-1 | mm | 63.6 | 9.4 | −1.4 | 39 | 77 | 0.1583 |
| Length of primary root (d14) | gpa1-2 | mm | 64.2 | 7.4 | −1.2 | 35 | 73 | 0.2153 |
| Length of primary root (d8) | Col | mm | 9.8 | 1.5 | n.a. | 9 | n.a. | n.a. |
| Length of primary root (d8) | agb1-1 | mm | 9.2 | 1.8 | −0.9 | 19 | 26 | 0.3741 |
| Length of primary root (d8) | agb1-2 | mm | 12.5 | 1.4 | 4.0 | 9 | 16 | 0.0010 |
| Length of primary root (d8) | WS | mm | 8.8 | 1.2 | n.a. | 10 | n.a. | n.a. |
| Length of primary root (d8) | gpa1-1 | mm | 8.4 | 1.8 | −0.5 | 10 | 18 | 0.6132 |
| Length of primary root (d8) | gpa1-2 | mm | 8.7 | 1.6 | −0.1 | 10 | 18 | 0.9272 |
| Maximum rosette radius | Col | mm | 51.1 | 6.5 | n.a. | 19 | n.a. | n.a. |
| Maximum rosette radius | agb1-1 | mm | 45.2 | 2.7 | −2.6 | 9 | 26 | 0.0141 |
| Maximum rosette radius | agb1-2 | mm | 42.2 | 4.0 | −4.0 | 10 | 27 | 0.0005 |
| Maximum rosette radius | WS | mm | 50.6 | 4.7 | n.a. | 18 | n.a. | n.a. |
| Maximum rosette radius | gpa1-1 | mm | 45.6 | 5.2 | −2.6 | 10 | 26 | 0.0143 |
| Maximum rosette radius | gpa1-2 | mm | 44.7 | 3.3 | −3.5 | 10 | 26 | 0.0015 |
| Number of abnormal seeds/half silique | Col | count | 0.0 | 0.0 | n.a. | 14 | n.a. | n.a. |
| Number of abnormal seeds/half silique | agb1-1 | count | 0.2 | 0.3 | 1.9 | 9 | 22 | 0.0691 |
| Number of abnormal seeds/half silique | agb1-2 | count | 0.0 | 0.0 | 0.0 | 10 | 22 | 1.0000 |
| Number of abnormal seeds/half silique | WS | count | 0.0 | 0.0 | n.a. | 14 | n.a. | n.a. |
| Number of abnormal seeds/half silique | gpa1-1 | count | 0.0 | 0.0 | 0.0 | 8 | 20 | 1.0000 |
| Number of abnormal seeds/half silique | gpa1-2 | count | 0.0 | 0.0 | 0.0 | 8 | 20 | 1.0000 |
| Number of bolts >1 cm | Col | count | 5.5 | 0.7 | n.a. | 19 | n.a. | n.a. |
| Number of bolts >1 cm | agb1-1 | count | 5.0 | 0.5 | −2.0 | 9 | 26 | 0.0534 |
| Number of bolts >1 cm | agb1-2 | count | 6.0 | 0.9 | 1.5 | 10 | 27 | 0.1352 |
| Number of bolts >1 cm | WS | count | 6.5 | 2.6 | n.a. | 18 | n.a. | n.a. |
| Number of bolts >1 cm | gpa1-1 | count | 4.8 | 0.9 | −2.0 | 10 | 26 | 0.0592 |
| Number of bolts >1 cm | gpa1-2 | count | 5.0 | 0.8 | −1.8 | 10 | 26 | 0.0915 |
| Number of normal seeds/half silique | Col | count | 29.5 | 3.9 | n.a. | 14 | n.a. | n.a. |
| Number of normal seeds/half silique | agb1-1 | count | 19.9 | 1.9 | −6.9 | 9 | 21 | 0.0000 |
| Number of normal seeds/half silique | agb1-2 | count | 22.7 | 1.8 | −5.1 | 10 | 22 | 0.0000 |
| Number of normal seeds/half silique | WS | count | 26.2 | 7.9 | n.a. | 14 | n.a. | n.a. |
| Number of normal seeds/half silique | gpa1-1 | count | 30.6 | 3.5 | 1.5 | 8 | 20 | 0.1573 |
| Number of normal seeds/half silique | gpa1-2 | count | 30.5 | 4.1 | 1.4 | 8 | 20 | 0.1720 |
| Number of open flowers | Col | count | 13.4 | 7.8 | n.a. | 19 | n.a. | n.a. |
| Number of open flowers | agb1-1 | count | 6.7 | 7.4 | −2.2 | 9 | 26 | 0.0402 |
| Number of open flowers | agb1-2 | count | 7.5 | 5.0 | −2.1 | 10 | 27 | 0.0410 |
| Number of open flowers | WS | count | 14.7 | 14.8 | n.a. | 18 | n.a. | n.a. |
| Number of open flowers | gpa1-1 | count | 14.9 | 9.9 | 0.0 | 10 | 26 | 0.9732 |
| Number of open flowers | gpa1-2 | count | 4.6 | 4.5 | −2.1 | 10 | 26 | 0.0461 |
| Number of senescent flowers | Col | count | 15.8 | 6.7 | n.a. | 19 | n.a. | n.a. |
| Number of senescent flowers | agb1-1 | count | 5.8 | 5.8 | −3.9 | 9 | 26 | 0.0006 |
| Number of senescent flowers | agb1-2 | count | 11.6 | 10.8 | −1.3 | 10 | 27 | 0.1996 |
| Number of senescent flowers | WS | count | 19.9 | 11.8 | n.a. | 18 | n.a. | n.a. |
| Number of senescent flowers | gpa1-1 | count | 15.9 | 6.5 | −1.0 | 10 | 26 | 0.3282 |
| Number of senescent flowers | gpa1-2 | count | 6.1 | 5.8 | −3.5 | 10 | 26 | 0.0019 |
| Number of siliques | Col | count | 289.5 | 75.1 | n.a. | 19 | n.a. | n.a. |
| Number of siliques | agb1-1 | count | 497.6 | 86.5 | 6.5 | 9 | 26 | 0.0000 |
| Number of siliques | agb1-2 | count | 339.8 | 64.5 | 1.8 | 10 | 27 | 0.0838 |
| Number of siliques | WS | count | 472.1 | 124.0 | n.a. | 18 | n.a. | n.a. |
| Number of siliques | gpa1-1 | count | 439.6 | 72.4 | −0.8 | 10 | 26 | 0.4560 |
| Number of siliques | gpa1-2 | count | 446.2 | 75.0 | −0.6 | 10 | 26 | 0.5538 |
| Number of stem branches | Col | count | 2.8 | 0.6 | n.a. | 19 | n.a. | n.a. |
| Number of stem branches | agb1-1 | count | 1.7 | 0.5 | −4.7 | 9 | 26 | 0.0001 |
| Number of stem branches | agb1-2 | count | 2.6 | 1.2 | −0.6 | 10 | 27 | |
| Number of stem branches | WS | count | 4.2 | 0.8 | n.a. | 18 | n.a. | n.a. |
| Number of stem branches | gpa1-1 | count | 3.1 | 0.6 | −3.9 | 10 | 26 | 0.0006 |
| Number of stem branches | gpa1-2 | count | 3.6 | 0.5 | −2.2 | 10 | 26 | 0.0378 |
| Rosette dry weight (stage 6.0) | Col | g | 0.1033 | 0.0360 | n.a. | 10 | n.a. | n.a. |
| Rosette dry weight (stage 6.0) | agb1-1 | g | 0.1319 | 0.0204 | 1.6 | 5 | 13 | 0.1268 |
| Rosette dry weight (stage 6.0) | agb1-2 | g | 0.0951 | 0.0191 | −0.5 | 5 | 13 | 0.6465 |
| Rosette dry weight (stage 6.0) | WS | g | 0.1011 | 0.0278 | n.a. | 10 | n.a. | n.a. |
| Rosette dry weight (stage 6.0) | gpa1-1 | g | 0.0814 | 0.0466 | −1.0 | 5 | 13 | 0.3196 |
| Rosette dry weight (stage 6.0) | gpa1-2 | g | 0.1291 | 0.0346 | 1.6 | 4 | 12 | 0.1360 |
| Rosette leaves >1 mm in length | Col | count | 9.5 | 1.9 | n.a. | 19 | n.a. | n.a. |
| Rosette leaves >1 mm in length | agb1-1 | count | 12.4 | 0.7 | 4.5 | 9 | 26 | 0.0001 |
| Rosette leaves >1 mm in length | agb1-2 | count | 9.0 | 0.9 | −0.7 | 10 | 27 | 0.4665 |
| Rosette leaves >1 mm in length | WS | count | 10.7 | 1.2 | n.a. | 18 | n.a. | n.a. |
| Rosette leaves >1 mm in length | gpa1-1 | count | 9.8 | 1.0 | −1.9 | 10 | 26 | 0.0642 |
| Rosette leaves >1 mm in length | gpa1-2 | count | 9.7 | 0.7 | −2.4 | 10 | 26 | 0.0262 |
| Seed - Area | Col | mm² | 0.0860 | 0.0094 | n.a. | 18 | n.a. | n.a. |
| Seed - Area | agb1-1 | mm² | 0.0970 | 0.0047 | 3.3 | 9 | 25 | 0.0031 |
| Seed - Area | agb1-2 | mm² | 0.0872 | 0.0061 | 0.3 | 10 | 26 | 0.7318 |
| Seed - Area | WS | mm² | 0.0929 | 0.0072 | n.a. | 18 | n.a. | n.a. |
| Seed - Area | gpa1-1 | mm² | 0.0866 | 0.0054 | −2.4 | 10 | 26 | 0.0256 |
| Seed - Area | gpa1-2 | mm² | 0.0855 | 0.0079 | −2.5 | 10 | 26 | 0.0194 |
| Seed - Eccentricity | Col | n.a. | 0.81 | 0.03 | n.a. | 18 | n.a. | n.a. |
| Seed - Eccentricity | agb1-1 | n.a. | 0.73 | 0.02 | −7.1 | 9 | 25 | 0.0000 |
| Seed - Eccentricity | agb1-2 | n.a. | 0.76 | 0.02 | −5.1 | 10 | 26 | 0.0000 |
| Seed - Eccentricity | WS | n.a. | 0.82 | 0.02 | n.a. | 18 | n.a. | n.a. |
| Seed - Eccentricity | gpa1-1 | n.a. | 0.79 | 0.02 | −4.1 | 10 | 26 | 0.0004 |
| Seed - Eccentricity | gpa1-2 | n.a. | 0.78 | 0.03 | −5.1 | 10 | 26 | 0.0000 |
| Seed - Major axis | Col | mm | 0.4337 | 0.0180 | n.a. | 18 | n.a. | n.a. |
| Seed - Major axis | agb1-1 | mm | 0.4300 | 0.0096 | −0.6 | 9 | 25 | 0.5682 |
| Seed - Major axis | agb1-2 | mm | 0.4151 | 0.0117 | −2.9 | 10 | 26 | 0.0068 |
| Seed - Major axis | WS | mm | 0.4580 | 0.0166 | n.a. | 18 | n.a. | n.a. |
| Seed - Major axis | gpa1-1 | mm | 0.4254 | 0.0108 | −5.6 | 10 | 26 | 0.0000 |
| Seed - Major axis | gpa1-2 | mm | 0.4176 | 0.0184 | −5.9 | 10 | 26 | 0.0000 |
| Seed - Minor axis | Col | mm | 0.2522 | 0.0194 | n.a. | 18 | n.a. | n.a. |
| Seed - Minor axis | agb1-1 | mm | 0.2881 | 0.0102 | 5.2 | 9 | 25 | 0.0000 |
| Seed - Minor axis | agb1-2 | mm | 0.2675 | 0.0136 | 2.2 | 10 | 26 | 0.0361 |
| Seed - Minor axis | WS | mm | 0.2591 | 0.0136 | n.a. | 18 | n.a. | n.a. |
| Seed - Minor axis | gpa1-1 | mm | 0.2593 | 0.0119 | 0.0 | 10 | 26 | 0.9709 |
| Seed - Minor axis | gpa1-2 | mm | 0.2607 | 0.0170 | 0.3 | 10 | 26 | 0.7879 |
| Seed - Perimeter | Col | mm | 1.5000 | 0.0760 | n.a. | 18 | n.a. | n.a. |
| Seed - Perimeter | agb1-1 | mm | 1.5267 | 0.0343 | 1.0 | 9 | 25 | 0.3290 |
| Seed - Perimeter | agb1-2 | mm | 1.4700 | 0.0593 | −1.1 | 10 | 26 | 0.2916 |
| Seed - Perimeter | WS | mm | 1.6200 | 0.1048 | n.a. | 18 | n.a. | n.a. |
| Seed - Perimeter | gpa1-1 | mm | 1.5720 | 0.1179 | −1.1 | 10 | 26 | 0.2766 |
| Seed - Perimeter | gpa1-2 | mm | 1.5110 | 0.1067 | −2.6 | 10 | 26 | 0.0145 |
| Seed - S.D. radius | Col | n.a. | 19.6 | 1.9 | n.a. | 18 | n.a. | n.a. |
| Seed - S.D. radius | agb1-1 | n.a. | 14.7 | 1.0 | −7.4 | 9 | 25 | 0.0000 |
| Seed - S.D. radius | agb1-2 | n.a. | 16.2 | 1.1 | −5.2 | 10 | 26 | 0.0000 |
| Seed - S.D. radius | WS | n.a. | 20.5 | 1.4 | n.a. | 18 | n.a. | n.a. |
| Seed - S.D. radius | gpa1-1 | n.a. | 17.7 | 1.4 | −5.0 | 10 | 26 | 0.0000 |
| Seed - S.D. radius | gpa1-2 | n.a. | 17.2 | 2.0 | −5.2 | 10 | 26 | 0.0000 |
| Seed mass per plant - fresh | Col | g | 0.7144 | 0.0343 | n.a. | 18 | n.a. | n.a. |
| Seed mass per plant - fresh | agb1-1 | g | 0.7273 | 0.0298 | 1.0 | 9 | 25 | 0.3468 |
| Seed mass per plant - fresh | agb1-2 | g | 0.7776 | 0.0399 | 4.4 | 10 | 26 | 0.0002 |
| Seed mass per plant - fresh | WS | g | 0.7481 | 0.0656 | n.a. | 18 | n.a. | n.a. |
| Seed mass per plant - fresh | gpa1-1 | g | 0.8081 | 0.0473 | 2.5 | 10 | 26 | 0.0174 |
| Seed mass per plant - fresh | gpa1-2 | g | 0.8735 | 0.0521 | 5.2 | 10 | 26 | 0.0000 |
| Seed mass per plant - dry | Col | g | 0.7092 | 0.0323 | n.a. | 18 | n.a. | n.a. |
| Seed mass per plant - dry | agb1-1 | g | 0.7228 | 0.0292 | 1.1 | 9 | 25 | 0.2994 |
| Seed mass per plant - dry | agb1-2 | g | 0.7692 | 0.0379 | 4.4 | 10 | 26 | 0.0002 |
| Seed mass per plant - dry | WS | g | 0.7424 | 0.0635 | n.a. | 18 | n.a. | n.a. |
| Seed mass per plant - dry | gpa1-1 | g | 0.7970 | 0.0457 | 2.4 | 10 | 26 | 0.0244 |
| Seed mass per plant - dry | gpa1-2 | g | 0.8632 | 0.0505 | 5.2 | 10 | 26 | 0.0000 |
| Seedling fresh weight (d14) | Col | mg | 8.56 | 1.56 | n.a. | 4 | n.a. | n.a. |
| Seedling fresh weight (d14) | agb1-1 | mg | 8.21 | 0.70 | −0.4 | 4 | 6 | 0.6951 |
| Seedling fresh weight (d14) | agb1-2 | mg | 12.04 | 1.37 | 3.3 | 4 | 6 | 0.0155 |
| Seedling fresh weight (d14) | WS | mg | 8.88 | 0.45 | n.a. | 4 | n.a. | n.a. |
| Seedling fresh weight (d14) | gpa1-1 | mg | 7.28 | 0.45 | −5.0 | 4 | 6 | 0.0024 |
| Seedling fresh weight (d14) | gpa1-2 | mg | 7.38 | 0.35 | −5.3 | 4 | 6 | 0.0019 |
| Sepal length | Col | mm | 2.6 | 0.2 | n.a. | 14 | n.a. | n.a. |
| Sepal length | agb1-1 | mm | 1.9 | 0.1 | −12.1 | 9 | 21 | 0.0000 |
| Sepal length | agb1-2 | mm | 2.2 | 0.1 | −6.6 | 10 | 22 | 0.0000 |
| Sepal length | WS | mm | 1.9 | 0.1 | n.a. | 14 | n.a. | n.a. |
| Sepal length | gpa1-1 | mm | 2.1 | 0.1 | 4.1 | 8 | 20 | 0.0005 |
| Sepal length | gpa1-2 | mm | 2.3 | 0.2 | 7.1 | 10 | 22 | 0.0000 |
| Silique length | Col | mm | 15.2 | 1.3 | n.a. | 14 | n.a. | n.a. |
| Silique length | agb1-1 | mm | 11.5 | 0.9 | −7.3 | 9 | 21 | 0.0000 |
| Silique length | agb1-2 | mm | 11.7 | 0.3 | −8.0 | 10 | 22 | 0.0000 |
| Silique length | WS | mm | 14.5 | 2.5 | n.a. | 14 | n.a. | n.a. |
| Silique length | gpa1-1 | mm | 16.1 | 0.8 | 1.8 | 8 | 20 | 0.0896 |
| Silique length | gpa1-2 | mm | 16.2 | 1.9 | 1.8 | 9 | 21 | 0.0871 |
| Split siliques | Col | count | 6.9 | 4.4 | n.a. | 9 | n.a. | n.a. |
| Split siliques | agb1-1 | count | 6.6 | 2.8 | −0.2 | 9 | 16 | 0.8495 |
| Split siliques | agb1-2 | count | 6.4 | 3.7 | −0.3 | 10 | 17 | 0.7939 |
| Split siliques | WS | count | 2.3 | 2.5 | n.a. | 4 | n.a. | n.a. |
| Split siliques | gpa1-1 | count | 2.0 | 1.4 | −0.1 | 2 | 4 | 0.9053 |
| Total rosette area | Col | mm² | 3061.5 | 786.1 | n.a. | 10 | n.a. | n.a. |
| Total rosette area | agb1-1 | mm² | 3184.5 | 371.0 | 0.3 | 5 | 13 | 0.7485 |
| Total rosette area | agb1-2 | mm² | 2982.4 | 457.1 | −0.2 | 5 | 13 | 0.8400 |
| Total rosette area | WS | mm² | 2964.3 | 621.2 | n.a. | 10 | n.a. | na. |
| Total rosette area | gpa1-1 | mm² | 2457.7 | 1414.8 | −1.0 | 5 | 13 | 0.3429 |
| Total rosette area | gpa1-2 | mm² | 3434.6 | 948.4 | 1.1 | 4 | 12 | 0.2894 |
| Total rosette eccentricity | Col | n.a. | 0.48 | 0.09 | n.a. | 10 | n.a. | n.a. |
| Total rosette eccentricity | agb1-1 | n.a. | 0.47 | 0.11 | −0.2 | 5 | 13 | 0.8689 |
| Total rosette eccentricity | agb1-2 | n.a. | 0.39 | 0.13 | −1.7 | 5 | 13 | 0.1226 |
| Total rosette eccentricity | WS | n.a. | 0.53 | 0.12 | n.a. | 10 | n.a. | n.a. |
| Total rosette eccentricity | gpa1-1 | n.a. | 0.38 | 0.12 | −2.4 | 5 | 13 | 0.0335 |
| Total rosette eccentricity | gpa1-2 | n.a. | 0.43 | 0.13 | −1.5 | 4 | 12 | 0.1677 |
| Total rosette major axis | Col | mm | 85.0 | 9.7 | n.a. | 10 | n.a. | n.a. |
| Total rosette major axis | agb1-1 | mm | 77.3 | 1.7 | −1.8 | 5 | 13 | 0.1033 |
| Total rosette major axis | agb1-2 | mm | 73.9 | 5.5 | −2.4 | 5 | 13 | 0.0350 |
| Total rosette major axis | WS | mm | 85.7 | 7.0 | n.a. | 10 | n.a. | n.a. |
| Total rosette major axis | gpa1-1 | mm | 61.0 | 24.4 | −3.1 | 5 | 13 | 0.0092 |
| Total rosette major axis | gpa1-2 | mm | 75.7 | 12.4 | −1.9 | 4 | 12 | 0.0782 |
| Total rosette minor axis | Col | mm | 74.2 | 10.5 | n.a. | 10 | n.a. | n.a. |
| Total rosette minor axis | agb1-1 | mm | 67.5 | 4.3 | −1.3 | 5 | 13 | 0.2005 |
| Total rosette minor axis | agb1-2 | mm | 67.3 | 3.8 | −1.4 | 5 | 13 | 0.1809 |
| Total rosette minor axis | WS | mm | 71.4 | 7.9 | n.a. | 10 | n.a. | n.a. |
| Total rosette minor axis | gpa1-1 | mm | 56.6 | 23.2 | −1.9 | 5 | 13 | 0.0844 |
| Total rosette minor axis | gpa1-2 | mm | 67.2 | 8.0 | −0.9 | 4 | 12 | 0.3919 |
| Total rosette perimeter | Col | mm | 789.2 | 140.0 | n.a. | 10 | n.a. | n.a. |
| Total rosette perimeter | agb1-1 | mm | 585.4 | 62.1 | −3.1 | 5 | 13 | 0.0091 |
| Total rosette perimeter | agb1-2 | mm | 604.8 | 63.0 | −2.8 | 5 | 13 | 0.0160 |
| Total rosette perimeter | WS | mm | 748.1 | 112.9 | n.a. | 10 | n.a. | n.a. |
| Total rosette perimeter | gpa1-1 | mm | 422.2 | 186.9 | −4.3 | 5 | 13 | 0.0009 |
| Total rosette perimeter | gpa1-2 | mm | 524.2 | 81.4 | −3.6 | 4 | 12 | 0.0038 |
| Total rosette S.D. radius | Col | n.a. | 38.0 | 3.3 | n.a. | 10 | n.a. | n.a. |
| Total rosette S.D. radius | agb1-1 | n.a. | 28.9 | 5.6 | −4.0 | 5 | 13 | 0.0014 |
| Total rosette S.D. radius | agb1-2 | n.a. | 30.9 | 3.5 | −3.8 | 5 | 13 | 0.0020 |
| Total rosette S.D. radius | WS | n.a. | 36.5 | 3.9 | n.a. | 10 | n.a. | n.a. |
| Total rosette S.D. radius | gpa1-1 | n.a. | 28.7 | 1.8 | −4.2 | 5 | 13 | 0.0010 |
| Total rosette S.D. radius | gpa1-2 | n.a. | 25.2 | 2.8 | −5.2 | 4 | 12 | 0.0002 |

TABLE 2
Data from Early Plant Analysis and Phenomics Screens

| Description | Growth Stage | Control (Col-0) | agb1-1 | agb1-2 | Control (Ws) | gpa1-1 | gpa1-2 |
|---|---|---|---|---|---|---|---|
| Root emergence | Stage 0.5 | 5.3 | 5.1 | 5.1 | 5.0 | 5.0 | 5.3 |
| Hyptocotyl and cotyledon emergence | Stage 0.7 | 6.2 | 6.0 | 6.1 | 5.8 | 6.1 | 6.2 |
| Cotyledons fully open | Stage 1.0 | 7.7 | 7.2 | 8.4 | 7.8 | 8.2 | 8.4 |
| 2 rosette leaves | Stage 1.02 | 10.1 | 10.5 | 10.5 | 10.7 | 11.5 | 11.0 |
| 4 rosette leaves | Stage 1.04 | 14.0 | 14.0 | 14.0 | 14.0 | 14.0 | 14.0 |
| 10 rosette leaves | Stage 1.10 | 20.8 | 20.0 | 18.0 | 22.0 | 22.0 | 22.0 |
| First flower buds visible | Stage 5.10 | 19.5 | 24.1 | 18.5 | 22.2 | 22.0 | 22.0 |
| First flower open | Stage 6.00 | 27.2 | 28.1 | 25.4 | 27.6 | 28.0 | 28.0 |
| Flowering complete | Stage 6.90 | 42.9 | 48.0 | 42.7 | 44.2 | 42.3 | 44.4 |
| Root emergence | Stage 0.5 | 5.3 | 5.1 | 5.1 | 5.0 | 5.0 | 5.3 |
| Hyptocotyl and cotyledon emergence | Stage 0.7 | 0.9 | 1.0 | 1.0 | 0.8 | 1.1 | 0.9 |
| Cotyledons fully open | Stage 1.0 | 1.5 | 1.2 | 2.3 | 2.1 | 2.1 | 2.2 |
| 2 rosette leaves | Stage 1.02 | 2.4 | 3.3 | 2.1 | 2.9 | 3.3 | 2.6 |
| 4 rosette leaves | Stage 1.04 | 3.9 | 3.5 | 3.5 | 3.4 | 2.5 | 3.0 |
| 10 rosette leaves | Stage 1.10 | 6.8 | 6.0 | 4.0 | 8.0 | 8.0 | 8.0 |
| First flower buds visible | Stage 5.10 | 0.0 | 4.1 | 0.5 | 0.2 | 0.0 | 0.0 |
| First flower open | Stage 6.00 | 7.7 | 4.0 | 7.0 | 5.4 | 6.0 | 6.0 |
| Flowering complete | Stage 6.90 | 16.2 | 19.8 | 17.1 | 16.4 | 14.3 | 16.0 |
| Length of flowering period | | Control (Col-0) | agb1-1 | agb1-2 | Control (Ws) | gpa1-1 | gpa1-2 |
| | Mean | 16.2 | 19.8 | 17.1 | 16.4 | 14.3 | 16.0 |
| | T-Test | n.a. | 7.75 | 1.48 | n.a. | −2.66 | −0.41 |
| | P-value | n.a. | 1.89E−07 | 0.15 | n.a. | 1.78E−02 | 0.13 |

Representative phenotypic traits resulting from loss-of-function mutations in the Arabidopsis GPA1 gene are listed below.

1. Altered floral developmental progression—indicated by:

Decreased duration of flowering (gpa1-1)

2. Smaller, rounder seeds—indicated by:

Decreased seed area (gpa1-1 and gpa1-2)

Decreased seed eccentricity (gpa1-1 and gpa1-2)

Decreased seed major axis (gpa1-1 and gpa1-2)

Decreased seed perimeter (gpa1-2)

Decreased seed standard deviation of the radius (gpa1-1 and gpa1-2)

3. Increased fruit and seed yield—indicated by:

Increased biomass of siliques at growth stage 6.9 (gpa1-2)

Increased fresh and dry weight of seed per plant (gpa1-1 and gpa1-2)

4. Smaller, more dense rosette—indicated by:

Decreased rosette radius (gpa1-1 and gpa1-2)

Decreased rosette eccentricity (gpa1-1)

Decreased rosette major axis (gpa 1-1)

Decreased rosette perimeter (gpa1-1 and gpa1-2)

Decreased rosette standard deviation of the radius (gpa1-1 and gpa1-2)

Increased biomass of rosette at growth stage 6.9 (gpa1-1 and gpa1)

Representative phenotypic traits resulting from loss-of-function mutations in the Arabidopsis AGB1 gene are listed below.

1. Altered floral developmental progression—as indicated by:

Slower to first flower bud visable (agb1-1)

Slower to cessation of flowering (agb1-1)

Increased duration of flowering (agb1-1)

Faster to first flower opening (agb1-2)

2. Smaller, rounder rosette—as indicated by:

Decreased rosette radius (agb1-1 and agb1-2)

Decreased biomass of rosette at growth stage 6.9 (agb1-2)

Decreased rosette perimeter (agb1-1 and agb1-2)

Decreased rosette standard deviation of the radius (agb1-1 and agb1-2)

3. Increased reproductive biomass—as indicated by:

Increased biomass of siliques at growth stage 6.9 (agb1-1 and agb1-2)

Increased number of siliques per plant (agb1-1)

Increased fresh and dry weight of seed per plant (agb1-2)

4. Increased root biomass—as indicated by:

Increased number of lateral roots per seedling (agb1-2)

Increased length of primary root on day 8, 10 & 12 (agb1-2)

5. Larger, rounder seeds—as indicated by:

Increased seed area (agb1-1)

Increased fresh and dry weight of seed per plant (agb1-2)

Decreased seed eccentricity (agb1-1 and agb1-2)

Decreased seed major axis (agb1-2)

Increased seed minor axis (agb1-1 and agb1-2)

Decreased seed standard deviation of the radius (agb1-1 and agb1-2)

6. Other phenotypes:

Decreased number of stem branches (agb1-1)

### Example 2

### Root System Analysis of agb1 and gpa1 Mature Plants

The root system of agb1 and gpa1 mutant plants is shown in FIG. 2. The CoI-0 control, agb1-1, and agb1-2 and WS control, gpa1-1, and gpa1-2 plants were grown to maturity under a short-day (8:16 L:D) regimen at 23° C. for 3 weeks, then transferred to a long-day (16:8 L:D) regimen for an additional 2 weeks. Mature roots of the plants were scored. Special care was taken to ensure that no lateral root would be lost during soil removal. Mature roots of agb1 mutants developed more lateral roots than the CoI-0 control (FIG. 2A) and mature roots of gpa1 mutants developed fewer lateral roots than the WS control (FIG. 2B).

### Example 3

### Generation of Transgenic Plants Over-Expressing GPA1 (GOX) and AGB1 (BOX)

### Cloning

The full length Arabidopsis GPA1 and AGB1 cDNA coding region was cloned into binary vector pTA7002 (Aoyama & Chua (1997) Plant J. 11:605-612) for Agrobacterium-mediated transformation of Arabidopsis. GPA1* was made by changing an A to a T at position 1264 of GPA1 by site-directed mutagenesis (Kroll et al. (1992) J. Biol. Chem. 267:23183-23188) to create a Q to L change. The mutated cDNA was cloned into a pAS2-1 DNA binding domain vector (Clontech). The vector was introduced into the Agrobacterium strain GV3101 for agro-infection of Arabidopsis. Transgenic plants were selected from the T1 generation of agro-infected plants grown on plates containing hygromycin.

### RNA Quantification by Real Time PCR

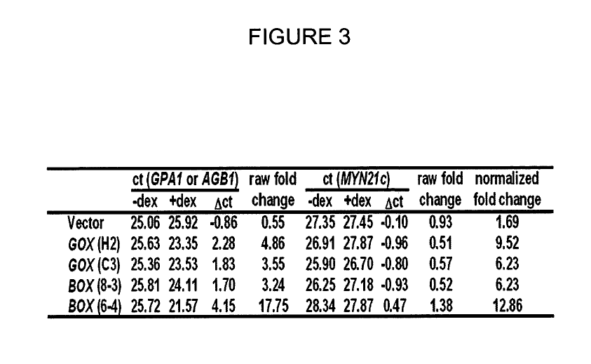

The GPA1 and AGB1 RNA expression levels of two independently transformed lines for each genotype were quantitated and the fold change over controls determined using quantitative PCR (FIG. 3). Total RNA from different transgenic lines was isolated from seedlings grown in light for 10 days with or without 100 nM of dexamethasone. 500 ng of total RNA was processed directly into cDNA by reverse transcription with Superscript II (Life Technologies) according to the manufacturer's protocol in a total volume of 20 μL. 1 μl of cDNA was used as a template for Real Time PCR analysis. Oligonucleotides were synthesized by Sigma-Genosys (Woodlands, Tex., US) using published sequence data from NCBI database. The primer sequences are:

| | | |
|---|---|---|
| GPA1 RT.FW | 5′ - AGAAGTTTGAGGAGTTATATTACCAG - 3′ | (SEQ ID NO:62) |
| GPA1 RT.RV | 5′ - AAGGCCAGCCTCCAGTAA - 3′ | (SEQ ID NO:63) |
| AGB1 RT.FW | 5′ - GACGTACTCGGGTGAGCTT - 3′ | (SEQ ID NO:64) |
| AGB1 RT.RV | 5′ - GAGCATTCCACACGATTAAT - 3′ | (SEQ ID NO:65) |

The primers were selected from the 3′ prime site of the gene to ensure the availability of transcripts from oligo (dT) based reverse transcription. The primers were expected to produce ˜150 bp products. Primers for a genomic marker MYN21c on the 5^{th} exon of sucrose cleavage protein-like gene were used as a control to normalize the expression data for each gene. The sequences of the control primers are listed below.

(SEQ ID NO:66):

| | |
|---|---|
| MYN21cF: | 5′ - CTAGCTTTGGAGTAAAAAGATTTGAG |
| | TGTGCAACC - 3′ |

(SEQ ID NO:67):

| | |
|---|---|
| MYN21cR: | 5′ - TCTTTTCGCTGTTTAATTGTAACCTT |
| | TGTTCTCGA - 3′ |

The primers are expected to produce a product of 333 bp from the control gene. PCR amplification and fluorescence detection was accomplished using the SMART CYCLER system of Cepheid Inc. (Sunnyvale, Calif.). SYBR green was used as the intercalating dye. The thermal cycling conditions were: 5 minutes in 96° C., followed by 40 cycles of 95° C. for 15 seconds, 60° C. for 15 seconds, and 72° C. for 15 seconds. The Primary Cycle Threshold (Cₜ) values were used to calculate difference of fold changes in treatments compared to the controls. The PCR cycle number at which the fluorescence from the PCR products reached 30 was taken as the Cₜ (Cycle Threshold) value for the corresponding reaction. A difference of 3.0 Cₜ equaled a 10-fold difference. Raw-fold change was calculated as 2^{ΔC}. Normalized-fold change was calculated by dividing the raw fold change in the treatment by the raw fold change in the control.

Seedlings of two transgenic GOX lines over-express GPA1 by a factor of 9.5 and 6.2 relative to the control.

### Example 4

Effect of Altered Expression of GPA1 and AGB1 on Lateral Root Formation in Plants

### Quantification of Lateral Root Primordia

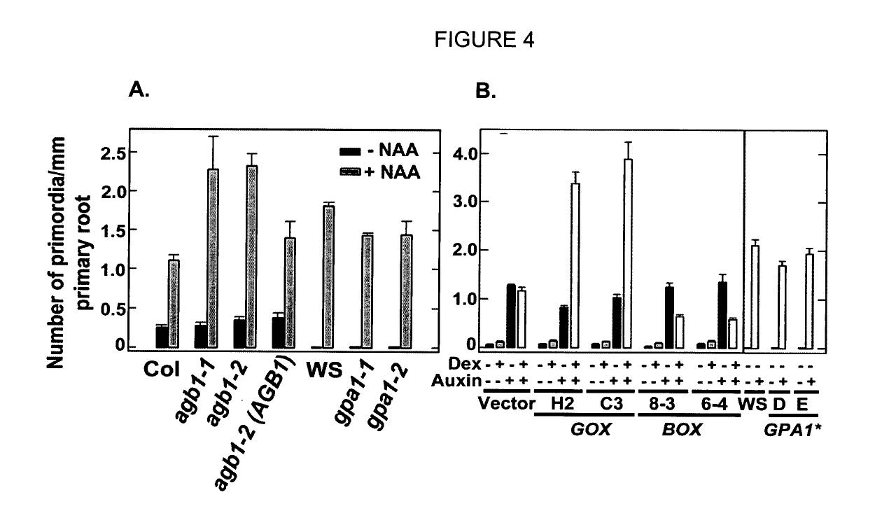

Quantification of lateral root primordia was performed using seedlings grown on media containing 5 μM of NPA (FIG. 4). After 9 days, seedlings were transferred to 1X MS media supplemented with or without 0.1 μM auxin and/or 100 nM dexamethasone as indicated in FIG. 4 and grown vertically under continuous light for four additional days. After clearing the tissues, root primordia were counted under Nomarski optics. The standard error of the mean is based on 10 seedlings. agb1 mutants developed more lateral roots than the CoI-0 control and transgenic gpa1 mutants developed fewer lateral roots than the WS control (FIG. 4A).

The roots of transgenic plants expressing GPA1 by a factor of 6-10 fold higher than wild-type exhibited an increased number of lateral root primordia relative to wild-type controls. This phenotype is dependent on the presence of the dexamethasone inducer and on the presence of exogenous auxin. The phenotype observed in plants that over-express GPA1 mimics that observed in the agb1 mutant background (Example 1 and FIG. 2).

### Example 5

### Construction of Driver Vectors

Vector construction: The bipartite transcription factor expressed by the driver lines is comprised of the yeast GAL4 DNA binding domain fused to two copies of the viral VP16 transcriptional activation domain and has been reported previously (GAL4/2XVP16; Schwechheimer et al. (1998) Plant Mol Biol 36: 195-240). A cassette containing the GAL4/2XVP16 open reading frame flanked by the doubled CaMV 35S promoter and the CaMV terminator (Schwechheimer et al. 1998) was cloned in a derivative of the binary vector pGPTV-HYG (Becker et al. (1992) Plant Mol Biol 20: 1195-1197) to make the constitutive driver construct pPG91. For tissue- or developmental-preferred expression of GAL4/2XVP16, sequences corresponding to the promoters below (except the two SLG13 promoters) were PCR amplified from CoI-0 DNA and used to replace the 2X 35S promoter sequence in pPG91. The SLG13 promoter sequences were PCR amplified from Brassica oleracea plants containing the S13 self-incompatibility haplotype. The promoters selected for this study were reported in: D1 (Prha)—Plesch et al. (1997) Plant J 12:635-647; D2 (AAP2)—Hirner et al. (1998) Plant J 14:535-544; D3 (Suc1)—Stadler et al. (1999) Plant J 19:269-278; D4 (Suc2)—Truernit & Sauer (1995) Planta 196:564-570; D5 (bZip)—Rook et al. (1998) Plant Mol Biol 37:171-178; D6 (VSP2)—Utsugi et al. (1998) Plant Mol Biol 38:565-576; D7 (ABI)—Giraudat et al. (1992) Plant Cell 10:1251-1261; D8 (FUS3)—Luerssen et al. (1988) Plant J 15:755-764; D9 (Oleosin)—Crowe et al. (2000) Plant Sci 151:171-181; D11 (GluB1)—Wen et al. (1989) Nucleic Acids Res 17:9490-9490; D12 (Em)—Finkelstein (1993) Mol Gen Genet. 238:401-408; D13 (AHA10)—Harper et al. (1994) Mol Gen Genet 244:572-587; D17 (Prp3)—Fowler et al. (1999) Plant Physiol 121:1081-1092; D18 (SLG13)—Dzelzkalns et al. (1993) Plant Cell 5:855-863; D19 (SLG13)—Dzelzkalns et al. (1993).

### Example 6

### Contruction of Target Vectors

Target genes for activation by the bipartite transcriptional activator were cloned in sense or antisense orientation behind a promoter consisting of 4 tandem copies of the GAL4 upstream activating sequence fused to the CaMV 35S minimal promoter (Schwechheimer et al. 1998) in a derivative of the binary vector pGPTV-BAR (Becker et al. 1992). The AGB1 genomic clone was PCR amplified with AGB1F (5′ GTTAATTMCTCAATCATGAACCTTCTTCTCTTCTA 3′) (SEQ ID NO:77) and AGB1R (5′ GGGCGCGCCGMGTTTAATTCTTCTAACCACTCCACTAT 3′) (SEQ ID NO:78) primers.

### Example 7

### Generation of Transgenic Plants

Generation of transgenic plants and crossing: The binary vectors were electro-transformed into Agrobacterium tumefaciens strain GV3101 and Arabidopsis plants were transformed by the floral dip method (Kloti and Mulpuri (2002) U.S. Pat. No. 6,353,155). Plant growth conditions were as described previously (Boyes et al. (2001) Plant Cell 13: 1499-1510). Driver constructs were transformed into wild-type CoI-0 plants. To assess the pattern of driver activity, hygromycin-resistant seedlings from each driver transformation were crossed with a line homozygous for the GUS target gene (pPG340). Hygromycin-resistant F1 progeny were allowed to self-pollinate and the resulting F2 generation was used for GUS expression analysis. Driver lines were selected for further development on the basis of strong and reproducible GUS staining patterns. The corresponding parental driver lines were made homozygous for crossing with target transgenes.

Target constructs containing AGB1 (antisense) were transformed into wild-type CoI-0. To generate lines with tissue-preferred transgene expression/suppression, reciprocal crosses were made between hemizygous target lines and homozygous drivers selected to produce the desired expression pattern. After 10 days of growth, Fl seedlings were sprayed with 1 ml/L of 18.19% glufosinate (Basta, AgrEvo USA Company) to select for the presence of the target transgene. In majority of cases the expected segregation ratio of 1:1 (Basta^{R}:Basta^{S}) was observed and 6 Basta^{R} seedlings were transferred to individual pots for further phenotypic analysis. As a positive control, the AGB1 target transgene was also transformed directly into CoI-0 plants homozygous for the constitutive 2X 35S/Gal4DBD/2XVP16 driver construct (pPG91).

### Example 8

### Glucuronidase (GUS) Assay

GUS activity was assayed using a protocol adapted from Malamy and Benfey (1997). Seedlings or excised tissues were vacuum infiltrated with a buffer containing 100 mM Tris-HCl (pH 7.5), 2.9 mg/ml NaCl, 0.66 mg/ml potassium ferricyanide, 20% (v/v) methanol, 0.001% (v/v) Triton X-100, and 0.5 μg/ml X-Gluc (Research Product International, Mt. Prospect, Ill.). After incubation for at least 16 hours at 37° C. in the dark, seedlings were cleared in 70% ethanol and observed under a MZ8 dissecting or DM LB compound microscope (Leica Microsystems, Wetzlar, Germany). A SPOT CCD digital camera (Diagnostic Instruments, MI) was used for image acquisition. Image analysis was performed by the SPOT (version 3.1) software.

### Example 9

### Root-Preferred Drivers for Transactivation

FIG. 5A illustrates a transactivation scheme for Arabidopsis. Seven root-preferred promoter sequences were chosen based on their preliminary expression patterns and used to control expression of the driver chimeric transactivating factor in a tissue-preferred manner. Three independent lines for each driver construct were crossed to a GUS target line and GUS expression in at least two F2 progeny lines was determined in all tissues at 8 defined stages from seedling to mature plant. Segregating F2 generations were used to monitor the reporter gene expression at growth stages 0.1 (seeds), 0.7 (5 days), 1.02 (10 days), 1.04 (15 days), 1.08 (20 days), 3.90 (30 days), 6.30 (40 days) and 8.00 (50 days) as designated by Boyes et al. (2001) Plant Cell 13:1499-1510. The most prominent, although not exclusive, expression location and stage for the driver constructs are provided in FIG. 5B. Expression among different independent transformant lines based on GUS activity did not vary and the expected segregation ratio of stained to non-stained seedlings was found in the F2 progeny (data not shown).

No phenotypes were found co-segregating with the driver or target lines indicating that by separating the two constructs, gene expression is latent or at a level that does not interfere with normal growth and development, that the promoter and gene insertions did not induce mutation, and that expression of Gal4 itself, as expected, does not interfere with normal activities.

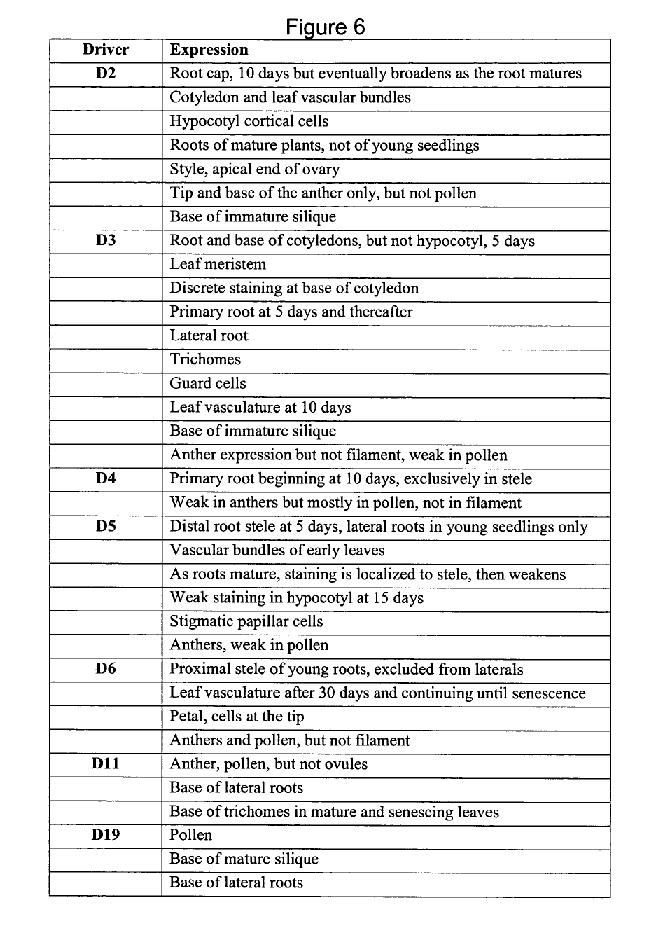

FIG. 6 provides a description of the spatial and temporal expression pattern provided by the promoters of the invention. Driver D2 utilizes the promoter for H+/amino acid permease gene expression. This gene was reported to be restricted to the vascular system of the silique (Hirner et al. (1998) Plant J. 14:535-544). D3 is based on the AtSuc promoter. This promoter was reported to drive expression in anther connective tissue, funiculi, and in mature pollen grains (Stadler et al. (1999) Plant J. 19:269-278). The expression patterns described herein for D2 and D3 were found in at least two of the three independent driver lines (data not shown).

### Example 10

### Separating Pleiotropic Phenotypes Using the Transactivation System

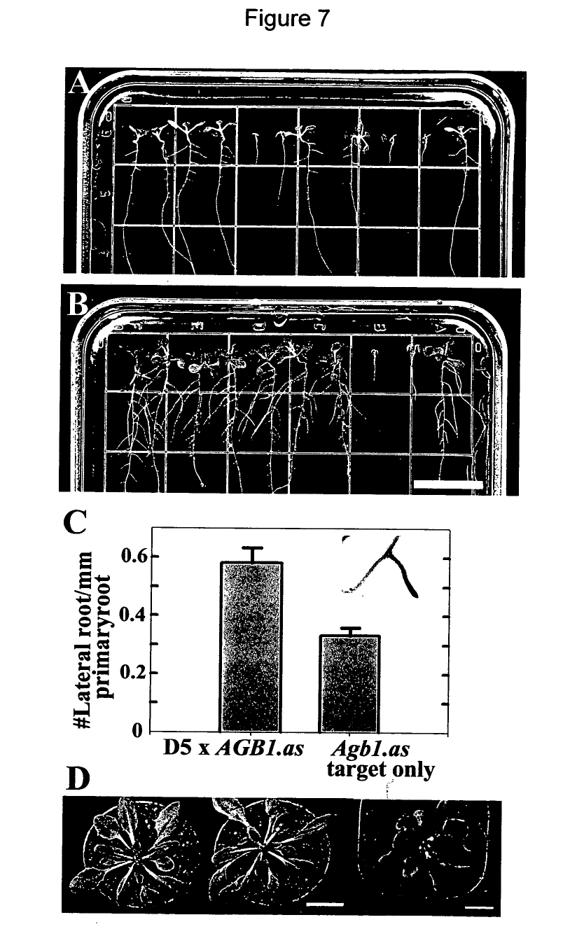

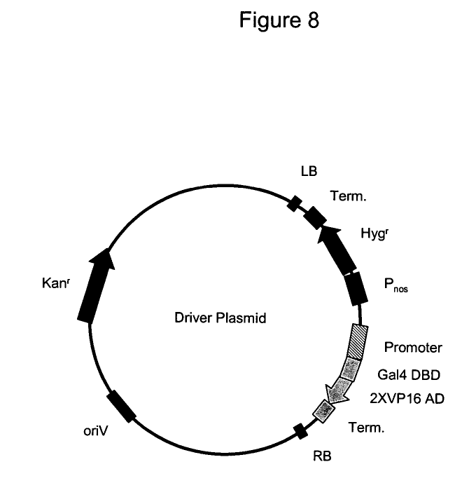

Transcript null mutants in the single gene encoding the beta subunit of a heterotrimeric G protein complex have many easily scored phenotypes (Ullah et al. (2003) Plant Cell, Volume 15, published Jan. 17, 2003, 10.1105/tpc.006148). Two are used here to illustrate the ability of the transactivation system to uncouple tissue-specific phenotypes. First, agb1 plants have a much larger root mass due primarily to increased lateral root number. In addition, agb1 mutants have rounded leaf lamina. In crosses between plants containing the target antisense construct AGB1.as and D5 (a driver that promotes root-preferred expression), 50% of the F1 progeny had increased root mass due to more lateral roots (FIG. 7A-C). In addition, none of the progeny had the rounded leaf phenotype observed in the agb1 null mutants. However, the progeny of crosses between AGB1.as and constitutive driver, PG91, displayed the expected rounded leaf phenotype (FIG. 7D).

Although the invention has been described with respect to a preferred embodiment thereof, it is also to be understood that it is not to be so limited since changes and modifications can be made therein which are within the full intended scope of the present invention. Furthermore, the foregoing description is for the purpose of illustration only, and not for the purpose of limitation, as the invention is defined by the claims as set forth hereinafter.

That which is claimed:

1. A method for altering a plant agronomic trait selected from the group consisting of time to flowering, duration of flowering in a plant, fruit yield, seed yield, root biomass, seed size, seed shape, number of stem branches, and size of a plant, the method comprising:
(a) introducing into a plant cell an expression cassette comprising a nucleotide sequence operably linked to a promoter that is operable within the plant cell, wherein the nucleotide sequence is selected from the group consisting of:
(i) a nucleotide sequence antisense to a plant AGB1 or an AGB1 ortholog,
(ii) a nucleotide sequence comprising an inverted repeat of AGB1 or an AGB1 ortholog,
(iii) a nucleotide sequence encoding a dsRNA, the dsRNA comprising a first RNA complementary to at least 25 consecutive nucleotides of a plant AGB1 or an AGB1 ortholog and a second RNA substantially complementary to the first RNA,
(iv) a nucleotide sequence that is AGB1 or an AGB1 ortholog, and
(v) a nucleotide sequence that is GPA1 or a GPA1 ortholog; and
(b) regenerating a plant that has a stably integrated expression cassette from the plant cell, wherein the regenerated plant has an altered agronomic trait.

2. The method of claim 1, wherein the promoter is selected from the group consisting of constitutive, inducible, developmentally regulated, tissue-preferred, minimal and 35S promoters.

3. The method of claim 1, wherein the plant is a dicot, a monocot, a gymnosperm or a member of the genus Brassica.

4. The method of claim 1, wherein the nucleotide sequence that is AGB1 has the sequence set forth in SEQ ID NO:1.

5. The method of claim 1, wherein the nucleotide sequence that is GPA1 has the sequence set forth in SEQ ID NO:3

6. The method of claim 1, wherein the altered plant agronomic trait is time to flowering, and the regenerated plant has an altered time to flowering.

7. The method of claim 1, wherein the altered plant agronomic trait is duration to flowering wherein the plant has an altered duration of flowering.

8. The method of claim 1, wherein the altered plant agronomic trait is fruit yield, and the regenerated plant has an altered fruit yield.

9. The method of claim 1, wherein the altered plant agronomic trait is seed yield, and the regenerated plant has an altered seed yield.

10. The method of claim 1, wherein the altered plant agronomic trait is altered seed size and the regenerated plant has an altered seed size

11. The method of claim 1, wherein the altered plant agronomic trait is seed shape and the regenerated plant has an altered seed shape.

12. The method of claim 1, wherein the altered plant agronomic trait is altered plant size, and the regenerated plant has an altered plant size.

13. The method of claim 1, wherein the altered plant agronomic trait is number of stem branches and the regenerated plant has an altered number of stem branches.

14. A method for altering a plant agronomic trait selected from the group consisting of time to flowering, duration of flowering in a plant, fruit yield, seed yield, root biomass, seed size, seed shape, number of stem branches, and size of a plant, the method comprising:
a) causing a disruption in a gene in a plant cell other than Arabidopsis, wherein the gene is an AGB1 ortholog endogenous to the plant cell; and
b) regenerating a plant from the plant cell, wherein the plant has a disruption in the endogenous gene and the plant exhibits an altered agronomic trait.

15. The method of claim 14, wherein the disruption is caused by a ribozyme complementary to the AGB1 ortholog.

16. The method of claim 14, wherein the disruption is caused by transposon or T-DNA insertion.

17. The method of claim 14, wherein the disruption is caused by site-directed mutagenesis.

18. The method of claim 14, wherein the disruption is caused by random mutagenesis.

19. A method for altering a plant agronomic trait selected from the group consisting of time to flowering, duration of flowering in a plant, fruit yield, seed yield, root biomass, seed size, seed shape, number of stem branches, and size of a plant, the method comprising:
a) causing a disruption in a gene in a plant cell that is not Arabidopsis thaliana or Orzya sativa, wherein the gene is a GPA1 ortholog endogenous to the plant cell; and
b) regenerating a plant from the plant cell, wherein the plant has a disruption in the endogenous gene and the plant exhibits an altered fruit and seed yield.

20. The method of claim 19, wherein the disruption is caused by a ribozyme complementary to the GPA1 ortholog.

21. The method of claim 19, wherein the disruption is caused by transposon or T-DNA insertion.

22. The method of claim 19, wherein the disruption is caused by site-directed mutagenesis.

23. The method of claim 19, wherein the disruption is caused by random mutagenesis.

24. A transgenic plant having stably integrated into its genome an expression cassette comprising a nucleotide sequence operably linked to a promoter that is operable within the plant, wherein the nucleotide sequence is selected from the group consisting of:
(a) a nucleotide sequence antisense to a nucleotide sequence that is AGB1 or an AGB1 ortholog,
(b) a nucleotide sequence comprising an inverted repeat of AGB1 or an AGB1 ortholog,
(c) a nucleotide sequence encoding a dsRNA, the dsRNA comprising a first RNA complementary to at least 25 consecutive nucleotides of a plant AGB1 or an AGB1 ortholog and a second RNA substantially complementary to the first RNA, and
(d) a nucleotide sequence that is AGB1 or an AGB1 ortholog.

25. The transgenic plant of claim 24, wherein the plant is a dicot, a monocot, a gymnosperm, a member of the genus Brassica, or Brassica napus.

26. Transgenic seed from the plant of claim 24.

27. A transgenic plant that is not Arabidopsis, wherein the plant has a disruption in a gene that is an AGB1 ortholog endogenous to the plant.

28. The transgenic plant of claim 27, wherein the plant is a dicot, a monocot, a gymnosperm, a member of the genus Brassica, or Brassica napus.

29. A transgenic plant having stably integrated into its genome an expression cassette comprising a nucleotide sequence operably linked to a promoter that is operable within the plant, wherein the nucleotide sequence is selected from the group consisting of:
i) a nucleotide sequence antisense to a nucleotide sequence that is GPA1 or a GPA1 ortholog,
ii) a nucleotide sequence comprising an inverted repeat of GPA1 or an GPA 1 ortholog,
iii) a nucleotide sequence encoding a dsRNA, the dsRNA comprising a first RNA complementary to at least 25 consecutive nucleotides of a plant GPA1 or an GPA1 ortholog and a second RNA substantially complementary to the first RNA, and
iv) a nucleotide sequence that is GPA1 or a GPA1 ortholog.

30. The transgenic plant of claim 29, wherein the plant is a dicot, a monocot, a member of the genus Brassica, or Brassica napus.

31. Transgenic seed from the plant of claim 29.

32. A transgenic plant that is not Arabidopsis thaliana or Orzya sativa, wherein the plant has a disruption in a gene that is a GPA1 ortholog endogenous to the plant.

33. The transgenic plant of claim 32, wherein the plant is a dicot, a monocot, a member of the genus Brassica, or Brassica napus.

34. Transgenic seed from the plant of claim 32.

35. A method for producing a transgenic plant having increased root biomass, comprising:
generating a transgenic plant comprising a driver cassette comprising
(a) a synthetic chimeric transcription factor open reading frame operably linked to a root-preferred promoter; and
(b) a target cassette comprising a nucleotide sequence in the antisense orientation operably linked to a minimal promoter operably linked to at least one cognate upstream activating sequence, wherein the nucleotide sequence in the antisense orientation is selected from the group consisting of (i) at least a portion of an AGB1 gene sequence set forth in SEQ ID NO:1 and (ii) at least a portion of an ortholog of an AGB1 gene sequence set forth in SEQ ID NO:1;
wherein each of the driver and the target cassettes is stably integrated in the genome of the plant and the plant has an increased root biomass.

36. The method according to claim 35, wherein the root-preferred promoter is a bZIP root-preferred promoter

37. The method according to claim 35, wherein the root-preferred promoter is a D5 bZIP promoter

38. The method according to claim 35, wherein the synthetic chimeric transcription factor open reading frame is a GAL4/VP16 open reading frame.

39. The method according to claim 35, wherein driver cassette comprises a GAL4/VP16 open reading frame is operably linked to a bZIP root-preferred promoter.

40. The method according to claim 35, wherein at least one cognate upstream activating sequence is a GAL4 upstream activating sequence.

41. The method of claim 35, wherein the plant is selected from the group consisting of monocots, dicots, vegetable crops, tomato, potato, pea, spinach, tobacco, soybean, sunflower, peanut, alfalfa, mint, cotton, rice, maize, oats, wheat, barley, sorghum, grasses, Brassica, Brassica napus, and Arabidopsis.

42. A transgenic plant having increased root biomass, the plant comprising:
a) a driver cassette comprising a synthetic chimeric transcription factor open reading frame operably linked to a root-preferred promoter; and
b) a target cassette comprising a nucleotide sequence in the antisense orientation operably linked to a minimal promoter operably linked to at least one cognate upstream activating sequence;
wherein the nucleotide sequence is selected from the group consisting of: (i) at least a portion of an AGB1 gene sequence set forth in SEQ ID NO:1 and (ii) at least a portion of an ortholog of an AGB1 gene sequence set forth in SEQ ID NO:1; and
wherein the driver cassette and target cassette are stably integrated into the plant genome.

43. The transgenic plant of claim 42, wherein the synthetic chimeric transcription factor open reading frame is a GAL4/VP16 open reading frame.

44. The transgenic plant of claim 42, wherein the root-preferred promoter is bZIP root-preferred promoter

45. The transgenic plant of claim 42, wherein the root-preferred promoter is a D5 bZIP promoter

46. The transgenic plant of claim 42, wherein at least one cognate upstream activating sequence is a GAL4 upstream activating sequence.

47. The transgenic plant of claim 42, wherein the driver cassette comprising a GAL4/VP16 open reading frame is operably linked to a D5 bZIP promoter.

48. The transgenic plant of claim 42, wherein the target cassette comprising at least a portion of an AGB1 gene sequence set forth in SEQ ID NO:1 in the antisense orientation is operably linked to a minimal promoter operably linked to at least one GAL4 upstream activating sequence.

49. The transgenic plant of claim 42, wherein the plant is selected from the group consisting of monocots, dicots, vegetable crops, tomato, potato, pea, spinach, tobacco, soybean, sunflower, peanut, alfalfa, mint, cotton, rice, maize, oats, wheat, barley, sorghum, grasses, Brassica, Brassica napus, and Arabidopsis.

50. Transgenic seed of the plant of claim 42.
